SEQUENCE LISTING (16 sequences)

SEQ ID NO: 1 - DNA, 18 bp, Artificial Sequence
  primer
gtggacctggctgaggag

SEQ ID NO: 2 - DNA, 20 bp, Artificial Sequence
  primer
ctttcaatcggggatgtctg

SEQ ID NO: 3 - DNA, 20 bp, Artificial Sequence
  primer
atgtgcgcttccagcaaatc

SEQ ID NO: 4 - DNA, 20 bp, Artificial Sequence
  primer
accacaggaaggaccagagt

SEQ ID NO: 5 - DNA, 23 bp, Artificial Sequence
  primer
tcagtaccttagggaagctgaaa

SEQ ID NO: 6 - DNA, 19 bp, Artificial Sequence
  primer
ccagtccaccagaatcgtg

SEQ ID NO: 7 - DNA, 20 bp, Artificial Sequence
  primer
ttcagtgcctaacagtggct

SEQ ID NO: 8 - DNA, 24 bp, Artificial Sequence
  primer
gaatgtccaaaaacgactcatgct

SEQ ID NO: 9 - DNA, 25 bp, Artificial Sequence
  primer
caacctcttcagtagaaggtaaaca

SEQ ID NO: 10 - DNA, 20 bp, Artificial Sequence
  primer
tgccaccaactatccagacc

SEQ ID NO: 11 - DNA, 20 bp, Artificial Sequence
  primer
tgcccctacagtcactttcc

SEQ ID NO: 12 - DNA, 21 bp, Artificial Sequence
  primer
agcaggcattttctatgttgc

SEQ ID NO: 13 - DNA, 20 bp, Artificial Sequence
  primer
aggggcacctgattggtgat

SEQ ID NO: 14 - DNA, 20 bp, Artificial Sequence
  primer
cagccactgtttctgggttg

SEQ ID NO: 15 - DNA, 22 bp, Artificial Sequence
  primer
ggcaaaccagaactaaggaaaa

SEQ ID NO: 16 - DNA, 22 bp, Artificial Sequence
  primer
ttcgatatgactttcgttgtcg

(19) United States
(12) Patent Application Publication
(10) Pub. No.: US 2023/0019818 A1
(43) Pub. Date: Jan. 19, 2023

(54) METHODS OF DETECTING AND TREATING HPV-POSITIVE HEAD AND NECK SQUAMOUS CELL CARCINOMA

(71) Applicant: The University of North Carolina at Chapel Hill, Chapel Hill, NC (US)

(72) Inventors: Antonio Amelio, Pittsboro, NC (US); Chad V. Pecot, Pittsboro, NC (US)

(21) Appl. No.: 17/851,545

(22) Filed: Jun. 28, 2022

Related U.S. Application Data
(60) Provisional application No. 63/215,674, filed on Jun. 28, 2021.

(51) Int. Cl. C12Q 1/6886; G01N 33/574

(52) U.S. Cl. CPC C12Q 1/6886; G01N 33/57407; C12Q 1/6851

(57) ABSTRACT

The present invention relates to methods of diagnosing and/or treating head and neck squamous cell carcinoma (HNSCC) in a subject. The present invention further relates to methods of diagnosing and/or treating head and neck squamous cell carcinoma (HNSCC) in a subject, wherein the subject has been diagnosed as being positive for human papillomavirus (HPV

### PRIORITY STATEMENT

This application claims the benefit, under 35 U.S.C. § 119(e), of U.S. Provisional Application Ser. No. 63/215,674, filed Jun. 28, 2021, the entire contents of which are incorporated by reference herein.

### STATEMENT OF GOVERNMENT SUPPORT

This invention was made with government support under Grant Number CA215075 awarded by the National Institutes of Health. The government has certain rights in the invention.

### STATEMENT REGARDING ELECTRONIC FILING OF A SEQUENCE LISTING

A Sequence Listing in ASCII text format, submitted under 37 C.F.R. § 1.821, entitled 5470-907_ST25.txt, 3,209 bytes in size, generated on Jun. 28, 2022 and filed via EFS-Web, is provided in lieu of a paper copy. This Sequence Listing is hereby incorporated herein by reference into the specification for its disclosures.

### FIELD OF THE INVENTION

The present invention relates to methods of diagnosing and/or treating head and neck squamous cell carcinoma (HNSCC) in a subject. The present invention further relates to methods of diagnosing and/or treating HNSCC in a subject, wherein the subject has been diagnosed as being positive for human papillomavirus (HPV).

### BACKGROUND OF THE INVENTION

Over 70% of oropharyngeal head and neck squamous cell carcinoma (HNSCC) cases in the United States are positive for human papillomavirus (HPV) yet biomarkers for stratifying oropharyngeal head and neck squamous cell carcinoma (HNSCC) patient risk are limited.

Head and neck squamous cell carcinomas are the sixth most common cancer worldwide (1-6). Patients with HNSCC are frequently treated with combinatorial therapy consisting of surgery and adjuvant radiation or chemoradiation, or upfront chemoradiation. Radiation treatment-associated morbidities, including loss of taste, reduced salivary flow, and swallowing dysfunction, can be permanent and can substantially impact patient quality of life.

The present invention addresses shortcomings in the art by providing methods of diagnosing and/or treating head and neck squamous cell carcinoma.

### SUMMARY OF THE INVENTION

One aspect of the present invention relates to a method of diagnosing head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a level of expression of the SYNGR3 gene and/or gene product from a sample comprising one or more immune cells obtained from a control subject (e.g., a control sample); and d) diagnosing the subject as being positive for human papillomavirus (HPV) when the level of expression of the SYNGR3 gene and/or gene product is greater than the level of expression of the SYNGR3 gene and/or gene product of the control subject.

Another aspect of the present invention provides a method of diagnosing and treating head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a level of expression of the SYNGR3 gene and/or gene product from a sample comprising one or more immune cells obtained from a control subject (e.g., a control sample); d) diagnosing the subject as being positive for human papillomavirus (HPV) when the level of expression of the SYNGR3 gene and/or gene product is greater than the level of expression of the SYNGR3 gene product of the control subject; and e) treating the HNSCC in the subject with a protocol for patients with that are infected by HPV-positive HNSCC.

Another aspect of the present invention provides a method of diagnosing and treating head and neck squamous cell carcinoma (HNSCC) in a subject, comprising treating the HNSCC in the subject with a protocol for patients with HPV-positive HNSCC if the subject is diagnosed as having an overexpression of SYNGR3 gene and/or gene product in one or more immune cells (e.g., T cell such as Th1 cell) in a HNSCC tumor of the subject.

Another aspect of the present invention provides a method of diagnosing head and neck squamous cell carcinoma (HNSCC) in a subject comprising: determining a SYNGR3 status of the subject based on a level of expression of the SYNGR3 gene and/or gene product in one or more immune cells (e.g., helper T cells) in a tumor of the subject; determining a p16 status of the subject based on a level of expression of a p16 gene and/or gene product in the tumor of the subject; and diagnosing the subject has having HPV-positive HNSCC based on the SYNGR3 status and the p16 status of the subject.

Another aspect of the present invention provides a method of diagnosing and treating head and neck squamous cell carcinoma (HNSCC) in a subject comprising: determining a SYNGR3 status of the subject based on a level of expression of the SYNGR3 gene and/or gene product in one or more immune cells (e.g., helper T cells) in a tumor of the subject; determining a p16 status of the subject based on a level of expression of a p16 gene and/or gene product in the tumor of the subject; diagnosing the subject has having HPV-positive HNSCC based on the SYNGR3 status and the p16 status of the subject; and if the subject is diagnosed as having HPV-positive HNSCC, treating the HNSCC in the subject with a protocol for patients with that have HPV-positive HNSCC.

Another aspect of the present invention provides a method of diagnosing head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a reference level of expression of the SYNGR3 gene and/or gene product (e.g., a level of expression of the SYNGR3 gene and/or gene product from a reference sample and/or a reference level as established in a reference database (e.g., a patient database)); and d) diagnosing the subject as being positive for human papillomavirus (HPV) when the level of expression of the SYNGR3 gene and/or gene product is greater than the reference level of expression of the SYNGR3 gene product.

Another aspect of the present invention provides a method of diagnosing and treating head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a reference level of expression of the SYNGR3 gene and/or gene product (e.g., a level of expression of the SYNGR3 gene and/or gene product from a reference sample and/or a reference level as established in a reference database (e.g., a patient database); d) diagnosing the subject as being positive for human papillomavirus (HPV) when the level of expression of the SYNGR3 gene and/or gene product is greater than the reference level of expression of the SYNGR3 gene and/or gene product; and e) treating the HNSCC in the subject with a protocol for patients with that are infected by HPV-positive HNSCC.

### BRIEF DESCRIPTION OF THE DRAWINGS

FIG. 1 shows a schematic of diagnostic approaches to HNSCC.

FIG. 2 shows data of HPV positive HNSCCs possessing unique immunogenomic signature. FIG. 2 panel A: TCGA RNAseq heatmap of the 112 most changed immune-related genes in the comparison HPV+ vs. HPV− for TCGA HNSCC patients (FDR-based analysis). FIG. 2 panel C: Similar heatmap as in panel B, analyses performed on only TH1 cells; SYNGR3 indicated in box.

FIG. 3 shows analyses indicated SYNGR3 is overexpressed in HNSCC samples positive for both p16 and HPV-ISH. qPCR validation data was performed in HNSCC human tumor samples. Analyses shown for p16 and HPV-ISH double positive (DP), double negative (DN), and p16-single-positive (SP) groups. Exemplar data plots show SYNGR3 mRNA is overexpressed in HNSCC samples positive for both p16 IHC and HPV-E6/E7 ISH.

FIG. 4 shows analyses indicating SYNGR3 protein is overexpressed in HNSCC samples positive for both p16 and HPV-E6/E7 ISH. Data labels as in FIG. 3.

FIG. 5 shows a schematic of the Scoring assignment for HPV status used in the cohort of HNSCC tumors (n=176) used in studies described in Example 1.

FIG. 6 shows example TMA-SYNGR staining in the cohort of HNSCC samples positive for both p16 and HPV-E6/E7 ISH. SYNGR3 protein was found to be overexpressed.

FIG. 7 shows example images of SYNGR protein expression in T-cells, as indicated with co-localization with immune cell marker CD3.

FIG. 8 shows a data plot quantifying percent of cell stained positive from imaging a shown in FIG. 7.

FIG. 9 shows a data plot quantifying percent of cell stained positive from imaging a shown in FIG. 7 in samples identified on average as DN, p16-SP, ISH-SP, or DP.

FIG. 10 shows a data plot quantifying ddPCR status corresponding to Table 1 for cells with medium-to-high SYNGR3 expression per TMA core.

FIG. 11 shows a representative data plot of SYNGR3 IHC sensitivity as predictive of HPV positivity, with indicated area under the curve (AUC) values.

FIG. 12 shows a second representative data plot of SYNGR3 IHC sensitivity as predictive of HPV positivity, with indicated area under the curve (AUC) values.

FIG. 13 shows two data plots comparing HPV marker staining (DN, SP-p16, SP-IGH, and DP) status for cells with medium-to-high SYNGR3 expression per TMA core, in cells categorized via staining performed with commercially available antibody from two different vendors.

FIG. 14 shows two data plots comparing ddPCR status for HPV-negative or HPV-positive for cells with medium-to-high SYNGR3 expression per TMA core, in cells categorized via staining performed with commercially available antibody from two different vendors.

FIG. 15 shows additional example images of co-expression of CD3 (immune cells) and SYNGR3.

FIG. 16 shows additional analyses of SYNGR3 expression in CD3+ cells in HSCC samples as quantified by comparative cell number (left) or percent of total cells (right).

FIG. 17 shows additional analyses of SYNGR3 expression in CD3+ cells in HSCC samples as quantified by comparative cell number (left) or percent of total cells (right), further delineated by DN, SP-p16, SP-ISH, or DP status.

FIG. 18 shows additional analyses of SYNGR3 expression in CD3+ cells in HSCC samples as quantified by comparative cell number (left) or percent of total cells (right), further delineated by DN, SP-p16, SP-ISH, or DP status in cells categorized via staining performed with commercially available antibody from two different vendors.

FIG. 19 shows additional analyses of SYNGR3 expression in CD3+ cells in HNSCC samples as quantified by comparative cell number (left) or percent of total cells (right), further delineated by ddPCR HPV-negative or HPV-positive status in cells categorized via staining performed with commercially available antibody from two different vendors.

FIG. 20 shows data plots in relation to HPV(+)HNSCC tumors exhibiting unique immunogenomic signatures associated with SYNGR3hi Th1 T cells. FIG. 20 panel A: Comparison of SYNGR3 expression according to squamous tumor type and HPV status. Data were extracted from the TCGA for HNSCC, CESC, ESCA, and LUSC RNA-seq datasets and log 2 median-centered expression plotted according to HPV status. TCGA=The Cancer Genome Atlas; HPV=human papillomavirus; HNSCC=head and neck squamous cell carcinoma; CESC=cervical and endocervical squamous cell carcinoma; ESCA=esophageal squamous cell carcinoma; LUSC=lung squamous cell carcinoma. FIG. 20 panel B: Comparison of SYNGR3 expression according to HPV status. Data for HPV(+) oropharyngeal squamous cell carcinomas (OPSCCs) and HPV(−) OPSCCs were extracted from the GSE65858 dataset and log 2 median-centered expression plotted according to HPV status. FIG. 20 panel C: Unsupervised hierarchical clustering of immune-related genes (n=1500) expressed in patients (n=109) from the HNSCC TCGA RNA-seq dataset. FIG. 20 panel D: Unsupervised hierarchical clustering of immune-related genes (n=8) expressed specifically in Th1 T cells of patients (n=109) from the HNSCC TCGA RNA-seq dataset. FIG. 20 panel E: Unsupervised hierarchical clustering of immune-related genes (n=8) expressed specifically in Th1 T cells of patients (n=109) from the CESC TCGA RNA-seq dataset.

FIG. 21 shows data plots and images in relation to validation of upregulated and downregulated genes in HPV(+) HNSCC patient tumors. Quantitative real-time PCR analysis of qPCR validation of genes found to be upregulated (FIG. 21 panel A) or downregulated (FIG. 21 panel B) in TCGA HNSCC RNA-seq data using fresh frozen whole human HNSCC patient tumors. Target gene expression was normalized to RPL23 mRNA levels and fold expression calculated relative to the average of the DN group. Data are presented as the mean±SEM (n=4 technical replicates). FIG. 21 panel C: Analysis of SYNGR3 protein expression in the fresh frozen tumor validation cohort. Representative 1× and high magnification 20× inset images from several independent ROIs of SYNGR3 IHC staining within the tumor stroma according to each respective HPV assay category.

FIG. 22 shows data plots and images in relation to validation of elevated SYNGR3 mRNA and protein in HPV(+) HNSCC patient tumors. FIG. 22 panel A: Schematic of fresh frozen human HNSCC patient tumors categorized by HPV assay clinical diagnoses, including p16 IHC and HPV16 ISH. DN=double negative (n=4), SP1=single positive for p16 IHC (n=3), DP=double positive for p16 IHC and HPV16 ISH (n=3). FIG. 22 panel B: Quantitative real-time PCR analysis of CDKN2A (gene name for p16 protein) mRNA levels. CDKN2A expression was normalized to RPL23 mRNA levels and fold expression was calculated relative to the average of the DN group. Data are presented as the mean±SEM (n=4 technical replicates; One-way ANOVA test, ns=not significant). FIG. 22 panel C: Quantitative real-time PCR analysis of SYNGR3 and CCNA1 mRNA levels. SYNGR3 and CCNA1 expression were normalized to RPL23 mRNA levels and fold expression was calculated relative to the average of the DN group. Data are presented as the mean±SEM (n=4 technical replicates; One-way ANOVA test, * P<0.05, ** P<0.01). FIG. 22 panel D: Analysis of SYNGR3 protein expression in the fresh frozen tumor validation cohort. Representative 1× and high magnification 20× inset images of SYNGR3 IHC staining within the tumor and stroma of sections according to each respective HPV assay category. FIG. 22 panel E: Quantification of SYNGR3 IHC staining in panel D represented as H-score. Data are presented as mean±SEM (* P<0.05).

FIG. 23 shows data plots and images in relation to antibody validation confirmation that SYNGR3 protein is expressed at significantly higher levels in HPV(+) HNSCC. FIG. 23 panel A: Schematic of TMA composed of formalin fixed paraffin embedded (FFPE) HNSCC patient tumor cores categorized by HPV assay clinical diagnoses, including p16 IHC and HPV16 ISH. DN=double negative (n=103), SP1=single positive for p16 IHC (n=69), SP2=single positive for HPV16 ISH (n=5), DP=double positive for p16 IHC and HPV16 ISH (n=13). Positive p16 IHC cores were defined as equal to or greater than 70% of cells with a score of 1, 2, or 3 in either the nucleus or cytoplasm. HPV ISH positive cores were defined as any cells with a nuclear score of 1, 2, or 3. FIG. 23 panel B: Comparison of SYNGR3 protein expression in HNSCC patient tumors. Representative images and high magnification 20× inset ROIs of SYNGR3 IHC staining depicting SYNGR3 expression within the tumor stroma shown for cores with the highest percentage of stained cells in each category of IHC scores (0=negative (no staining for SYNGR3); 1=low; 2=medium; 3=high). FIG. 23 panel C: Quantification of SYNGR3 IHC staining of CHANCE TMA by HPV assay category delineated in panel A represented as H-score. Data are presented as mean±SEM (*** P<0.001).

FIG. 24 shows data plots in relation to co-detection of SYNGR3 and p16 providing a tractable pair of IHC-only biomarkers for identifying HPV status in HNSCC. Receiver operating characteristic (ROC) curves plotting sensitivity by specificity of H-score (left) and percent positive cells (right, cells scoring 1-3) using two independent antibodies (FIG. 24 panels A and B) for SYNGR3 IHC staining of CHANCE TMA cases with known HPV status as determined by ddPCR. AUC=area under the curve.

FIG. 25 shows data plots and images in relation to T and B cells in the stromal compartment having the strongest correlation with high SYNGR3 expression. FIG. 25 panel A: Representative multiplex IHC staining of TMA cores for SYNGR3 (red), CD3 (cyan, pan T cell marker), pan-CK (green, pan-cytokeratin), and DAPI (purple, nuclei) with enumeration of SYNGR3+/CD3+ cells in CK+ and CK− regions. Scale bar=400 μm. FIG. 25 panel B: Representative multiplex IHC staining of TMA cores for SYNGR3 (red), CD45 (cyan, pan immune cell marker), p16 (green, tumor), and DAPI (purple, nuclei) with enumeration of SYNGR3+/CD3+ cells in p16+ and p16− regions. Scale bar=400 μm. FIG. 25 panel C: Quantification of IHC of SYNGR3 and CD3 (T cells; left) or SYNGR3 and CD45 (all immune cells; right) according to HPV assay category. Data represented as number of coexpressing cells in all ROIs. Expression data are presented as mean±SEM (*** P<0.001, **** P<0.0001). FIG. 25 panel D: Quantification of IHC of SYNGR3 and CD3 (T cells; left) or SYNGR3 and CD45 (all immune cells; right) according to HPV assay category separated by epithelial/stromal ROIs. Epithelial/tumor regions were defined by either pan-CK (left) or p16 IHC (right), and included the tumor margin analysis (defined by 25 μM on either side of tumor border). Data represented as density of coexpressing cells. Expression data are presented as mean±SEM (** P<0.01, *** P<0.001). FIG. 25 panels E and F: Single cell RNA-seq data of HNSCC HPV(+) tumors confirms SYNGR3 expression in T and B cells. Expression data are presented as UMAP plots.

FIG. 26 shows data plots and images in relation to additional multiplex staining coexpression and ROI quantification. FIG. 26 panel A: Quantification of IHC multiplex staining from FIG. 5A,C showing the percentage of total cells coexpressing SYNGR3 and CD3 (pan T cell marker) according to the different HPV assay categories in all regions of interest analyzed. Expression data are presented as mean±SEM (*** P<0.001). FIG. 26 panel B: Quantification of IHC multiplex staining of number of cells with SYNGR3 and CD3 (pan T cell) coexpression in samples with known HPV status established by ddPCR. Expression data are presented as mean±SEM (*** P<0.001). FIG. 26 panel C: Quantification of IHC multiplex staining showing number of cells expressing SYNGR3 within DP TMA cores, and highlighting cells with and without CD3 coexpression. FIG. 26 panel D: Top, representative multiplex IHC staining for SYNGR3 (red), CD4 (cyan, T helper cells), pan-CK (green, pan-cytokeratin), and DAPI (purple, nuclei) with dual positive SYNGR3+/CD4+ cells highlighted by yellow arrowheads in CK− regions. Bottom (FIG. 26 panel D), representative multiplex IHC staining for SYNGR3 (red), CD8 (cyan, cytotoxic T cells), pan-CK (green, pan-cytokeratin), and DAPI (purple, nuclei). Scale bar=400 μm. FIG. 26 panel E: Quantification of IHC of SYNGR3 and CD3 (T cells; left) or SYNGR3 and CD45 (all immune cells; right) according to HPV assay category and separated by epithelial/stromal regions of interest. Epithelial region defined by either pan-CK (left) or p16 IHC (right), and separating out the tumor margin (defined by 2504 on either side of tumor border). Data represented as density of coexpressing cells. Expression data are presented as mean±SEM.

FIG. 27 shows data plots in relation to the relationship between high SYNGR3 expression and Disease-Specific Survival of HPV(+) HNSCC patients. FIG. 27 panels A and B: Kaplan-Meier curves for overall survival (A) and disease specific survival (B) of TCGA HNSCC patients stratified by SYNGR3 expression from tumor RNA-seq data using UCSC Xenabrowser. High SYNGR3=top quartile mRNA expression, Low SYNGR3=bottom quartile mRNA expression; p value <0.05. FIG. 27 panel C: Probability of death in HPV(+) CHANCE TMA patients stratified according to SYNGR3 expression levels. (** P<0.01). FIG. 27 panel D: Probability of death in HPV(+) CHANCE TMA patients stratified by p16 cytoplasmic and nuclear expression by p16 IHC into localization categories. High cytoplasmic (HC)=cytoplasmic H-score of 50 and above; low cytoplasmic (LC)=cytoplasmic H score below 50; high nuclear (HN)=nuclear H-score of 70 and above; low nuclear (LN)=nuclear H-score below 70. (*** P<0.001). FIG. 27 panel E: Comparison of SYNGR3 protein expression by IHC stain of whole HPV(+) CHANCE TMA cores separated by the p16 localization categories defined in panel D. Expression data are presented as violin plot of H-score and presented as mean±SEM (**** P<0.0001). FIG. 27 panels F and G: Survival curves of following multivariate analysis of HPV(+) CHANCE TMA patients adjusted for age, sex, race, smoking status, alcohol status, and tumor site. Patients separated by p16 localization category defined in panel D. Unadjusted curves can be found in FIG. 28.

FIG. 28 shows data plots in relation to an association of SYNGR3 expression patient survival. FIG. 28 panel A: SYNGR3 mRNA levels from TCGA HNSCC patients stratified according to top and bottom quartile using UCSC Xenabrowser. High SYNGR3=top quartile mRNA expression, Low SYNGR 3=bottom quartile mRNA expression. (**** P<0.0001). FIG. 28 panels B and C: Survival curves of HPV(+) CHANCE TMA patients. Patients stratified by p16 cytoplasmic and nuclear expression by p16 IHC into localization categories. High cytoplasmic (HC)=cytoplasmic H-score of 50 and above; low cytoplasmic (LC)=cytoplasmic H score below 50; high nuclear (HN)=nuclear H-score of 70 and above; low nuclear (LN)=nuclear H-score below 70.

### DETAILED DESCRIPTION OF THE INVENTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

The term “and/or” when used in describing two or more items or conditions refers to situations where all named items or conditions are present or applicable, or to situations wherein only one (or less than all) of the items or conditions is present or applicable.

As used herein, the term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting essentially of”, and “consisting of”, where one of these three terms is used herein, the presently disclosed subject matter can include the use of either of the other two terms. For example, the presently disclosed subject matter relates in some embodiments to for detecting the presence of HPV infection in a subject, which methods comprise detecting the presence or absence of biomarker SYNGR3, and optionally biomarker p16, a sample from the subject. It is understood that the presently disclosed subject matter thus also encompasses methods that in some embodiments consist essentially of detecting the presence or absence of biomarker SYNGR3, and optionally biomarker p16, in a sample from the subject; as well as methods that in some embodiments consist of detecting the presence or absence of biomarker SYNGR3, and optionally biomarker p16, in a sample from the subject.

As used herein, HNSCC refers to “head and neck squamous cell carcinoma,” a cancer derived from the mucosal epithelium in the oral cavity, pharynx and larynx, including mucosal epithelium of the oral cavity (lips, buccal mucosa, hard palate, anterior tongue, floor of mouth and retromolar trigone), nasopharynx, oropharynx (palatine tonsils, lingual tonsils, base of tongue, soft palate, uvula and posterior pharyngeal wall), hypopharynx (the bottom part of the throat, extending from the hyoid bone to the cricoid cartilage) and larynx.

As used herein, the term “SYNGR3” refers to the Synaptogyrin 3 gene, as well as gene products encoded and/or derived therefrom.

As used herein, “HPV” refers to any human papillomavirus, including HPV strains 16, 18, 31, 33, 35, 39, 45, 51, 52, 56, 58, 59, 66, and 68. In particular embodiments, the HPV strain is HPV-16. As used herein, “positive for HPV” or “HPV-positive” means the subject has been infected with the HPV virus.

As used herein, “p16” refers to the p16 gene, as well as gene products encoded and/or derived therefrom.

As used herein, the term “HPV16 ISH” refers to in situ hybridization (ISH) comprising hybridization of a targeted nucleic acid probe to a portion of the HPV16 genome (e.g., a genotype-identifying (genotypic) portion). ISH is a known method of art in the field of the present invention, and upon review of the present disclosure, those skilled in the art will be familiar with numerous specific formats and variations of ISH that can be useful for carrying out the methods of the presently disclosed subject matter (e.g., RNA ISH, DNA ISH, HPV-E6/E7 ISH, single-cell ISH, polymerase chain reaction (PCR) ISH, fluorescence ISH (FISH), and the like). In some embodiments, the HPV16 portion identified by HPV16 ISH may be comprised within a sample (e.g., a patient sample, a tumor sample, and/or one or more isolated cells thereof), wherein successful HPV16 ISH may indicate the presence and/or expression level of HPV16 virus in the sample (e.g., “HPV16-positive”).

As used herein, an “immune cell” refers to a nucleated blood cell that lack hemoglobin and function in the immune system to protect against agents of infection and foreign matter. In some embodiments, the immune cell may be any cell which expresses CD3 antigen (“CD3-positive”). In particular embodiments, the immune cell is a lymphocyte, e.g., a T cell or a B cell. In some embodiments, the T cell is a helper T cell (e.g., T helper type 1 cell (“Th1”), Th2, Th3, Th9, Th17, etc.) In some embodiments, the T cell is a Th1 cell).

As used herein, a “gene product” refers to any material produced from the expression of a gene, including but not limited to RNA (e.g., mRNA) and proteins. In some embodiments, the gene product comprises one or more of a mRNA and a protein.

“Amino acid sequence” and terms such as “peptide”, “polypeptide”, and “protein” are used interchangeably herein, and are not meant to limit the amino acid sequence to the complete, native amino acid sequence (i.e., a sequence containing only those amino acids found in the protein as it occurs in nature) associated with the recited protein molecule.

The terms “antibody” and “immunoglobulin” include antibodies or immunoglobulins of any isotype, fragments of antibodies that retain specific binding to antigen, including but not limited to Fab, Fv, scFv, and Fd fragments, chimeric antibodies, humanized antibodies, single-chain antibodies, and fusion proteins including an antigen-binding portion of an antibody and a non-antibody protein. The antibodies can in some embodiments be detectably labeled, e.g., with a radioisotope, an enzyme which generates a detectable product, a fluorescent protein, and the like. The antibodies can in some embodiments be further conjugated to other moieties, such as members of specific binding pairs, e.g., biotin (member of biotin-avidin specific binding pair), and the like. Also encompassed by the terms are Fab′, Fv, F(ab′)2, and other antibody fragments that retain specific binding to antigen (e.g., any antibody fragment that comprises at least one paratope). Antibodies can exist in a variety of other forms including, for example, Fv, Fab, and (Fab′)₂, as well as bi-functional (i.e., bi-specific) hybrid antibodies (see e.g., Lanzavecchia et al., 1987) and in single chains (see e.g., Huston et al., 1988 and Bird et al., 1988, each of which is incorporated herein by reference in its entirety). See generally, Hood et al., 1984, and Hunkapiller & Hood, 1986.

As used herein, the term “sample” is used in its broadest sense. In one sense, it is meant to include a specimen from a biological source. Biological samples can be obtained from animals (including humans) and encompass fluids (e.g., blood, mucus, urine, saliva), solids, tissues, cells, and gases. In some embodiments, the sample is obtained from a tumor (e.g., tumor stroma) in the subject. The sample may also comprise one or more immune cells, including T cells of the subject, including immune cells (e.g., helper T cells) from the tumor (e.g., tumor stroma) of the subject.

A “subject” of the invention may include any animal in need thereof. In some embodiments, a subject may be, for example, a mammal, a reptile, a bird, an amphibian, or a fish. A mammalian subject may include, but is not limited to, a laboratory animal (e.g., a rat, mouse, guinea pig, rabbit, primate, etc.), a farm or commercial animal (e.g., cattle, pig, horse, goat, donkey, sheep, etc.), or a domestic animal (e.g., cat, dog, ferret, gerbil, hamster etc.). In some embodiments, a mammalian subject may be a primate, or a non-human primate (e.g., a chimpanzee, baboon, macaque (e.g., rhesus macaque, crab-eating macaque, stump-tailed macaque, pig-tailed macaque), monkey (e.g., squirrel monkey, owl monkey, etc.), marmoset, gorilla, etc.). In some embodiments, a mammalian subject may be a human. The terms “subject” and “patient” are in some embodiments used interchangeably herein, such as but not limited to in reference to a human subject or patient.

A “subject in need” of the methods of the invention can be any subject known or suspected to have a head and neck squamous cell carcinoma (HNSCC) and/or any SYNGR3-expressing tissue disorder and/or an illness to which the methods of the present invention disclosed herein may provide beneficial health effects, or a subject having an increased risk of developing the same.

As used herein the term “control” refers to a comparative sample and/or other reference source for a control subject.

“Control subject” as used herein refers to a subject which does not have said condition(s) of the subject in need, e.g., said head and neck squamous cell carcinoma (HNSCC) and/or said SYNGR3-expressing tissue disorder and/or an illness to which the methods of the present invention disclosed herein may provide beneficial health effects. In some embodiments, a control or control subject may be a reference level of expression of a gene product such as but not limited to SYNGR3, p16, or HPV16, wherein the reference level of expression of the gene product may be determined from a reference sample and/or determined from or as established in (i.e., predetermined by) a reference database (e.g., a preexisting database) such as any subject database (e.g., healthy subject and/or patient dataset)

As used herein, the term “dataset” refers to a collection of related sets of information, i.e., data, attained from experimental or computational analyses, comprising any type of data, including but not limited to healthy subject and/or patient data. The source material (e.g., healthy subject(s) and/or patient(s)) may be alternatively referred to as a database, a repository, a reference group, or any similar terminology understood in the art. A dataset may be screened and/or otherwise searched for particular data depending on variable parameters as defined by each particular dataset. In some embodiments, the dataset is gene product dataset, i.e., a dataset comprising gene products such as but not limited to mRNA and/or protein data.

As used herein, the term “biomarker” can mean any chemical or biological entity that is produced by cells (e.g., cells of the subject), or substances that are produced by cells that might be then chemically modified by extracellular enzymes, free radicals produced by cells of the body and/or other naturally occurring processes and that is found, for example, in the saliva, urine, blood, vaginal secretion, tears, feces, sputum, hair, nails, skin, wound fluid, nasal swab, lymph, perspiration, oral mucosa, vaginal mucosa, or the anus, or in serum or plasma obtained from blood. Thus, in the methods of this invention, the sample can be any biological fluid or tissue that can be used in an assay of this invention, including but not limited to, serum, plasma, blood, saliva, semen, lymph, cerebrospinal fluid, prostatic fluid, urine, sputum, oral mucosa, nasal mucosa, duodenal fluid, gastric fluid, skin, endothelium, biopsy material from a salivary gland, biopsy material of a parotid gland, biopsy material of other glands of the mouth, secretions of the salivary gland, secretions of the parotid gland, secretions of other glands of the mouth, joint fluid, body cavity fluid, tear fluid, anal secretions; vaginal secretions, perspiration, whole cells, cell extracts, tissue, biopsy material, aspirates, exudates, slide preparations, fixed cells, tissue sections, etc.

A biomarker of this invention can be detected and/or quantified (e.g., determination of biomarker presence and/or expression level) in a sample by a variety of methods well known in the art for detecting and/or quantifying substances in biological samples. For example, for detecting and/or quantifying a biomarker that is a peptide or polypeptide, standard methods for detecting and/or quantifying peptides and/or polypeptides in sample can be employed. Nonlimiting examples of such methods include direct protein measurement, absorbance at 280 nm, absorbance at 205 nm, extinction coefficient assay, Lowry assay, biuret assay, Bradford assay, bicinchoninic acid assay (BCA), amido black assay, colloidal gold assay, immunoassay or other specific binding assay employing an antibody or ligand that specifically binds a peptide or polypeptide, protein separation assays such as electrophoresis, gas chromatography (GC), high performance liquid chromatography (HPLC), mass spectrometry (MS), etc., as are well known in the art. Additional non-limiting examples of methods known in the art for the detection and/or quantification of biomarkers include, but are not limited to, polymerase chain reaction (PCR)-based techniques, gas chromatography (GC), liquid chromatography/mass spectroscopy (LC-MS), gas chromatography/mass spectroscopy (GC-MS), nuclear magnetic resonance (NMR), magnetic resonance imaging (MRI), Fourier Transform InfraRed (FT-IR), and inductively coupled plasma mass spectrometry (ICP-MS). It is further understood that mass spectrometry techniques include, but are not limited to, the use of magnetic-sector and double focusing instruments, transmission quadrapole instruments, quadrupole ion-trap instruments, time-of-flight instruments (TOF), Fourier transform ion cyclotron resonance instruments (FT-MS), and matrix-assisted laser desorption/ionization time-of-flight mass spectrometry (MALDI-TOF MS).

As used herein, the phrase “SYNGR3 status” refers to an assessment of SYNGR3 expression in a sample. The phrase “p16 status” refers to an assessment of p16 expression in a sample. The phrase “ISH status” refers to an assessment of the presence of HPV16 as determined by in situ hybridization. The “combined HPV status” refers to a determination of whether the subject is HPV positive as determined by the SYNGR3 status and one or more of p16 status and ISH status.

Multiple genomic and transcriptomic studies have profiled HNSCCs and identified distinct molecular subtypes that can be grouped in part based on human papilloma virus (HPV) status (7-9). Knowledge of HPV status has influenced the field of head and neck oncology and treatment planning given that patients with HPV(+) HNSCC have distinct clinicopathological features with significantly better prognosis compared to their HPV(−) counterparts (10-20). Unfortunately, the incidence and proportion of HPV(+) HNSCCs is rising (29-33) emphasizing the need to correctly identify patients with HPV associated disease. Correctly classifying HPV(+) HNSCC, as well as which of these patients may have improved prognosis and therefore may benefit from treatment de-escalation is a major focus of the oncology community, with an overarching goal of limiting the severity of treatment-induced side effects without reducing treatment efficacy (21-28).

Among the currently available HPV detection assays commonly used in clinical settings are those involving PCR of the viral oncogenes E6/E7, immunohistochemistry (IHC) of p16 protein, and in situ hybridization (ISH) of either DNA or RNA for E6/E7. These assays have varying reliability and availability depending upon sample requirements, test specificity and sensitivity, as well as presence of specific equipment and technical expertise (29). While PCR based detection methods of active HPV transcription are both sensitive and highly specific (29-33) and the FDA approved PCR to detect E6/E7 mRNA as the “gold standard” several years ago (34), widespread implementation of this approach has proven difficult. This may be attributable to several technical challenges (35-37), including dependence on fresh-frozen tumor tissue and in distinguishing between false negatives (e.g., arising from improper sample collection, specimen degradation, presence of PCR inhibitors) and false positives (e.g., detecting sample contaminants) making this approach impractical for routine use in many clinical locations (30, 38). In contrast, immunohistochemistry (IHC) detection of p16 overexpression in formalin fixed paraffin embedded (FFPE) tissues has proven to be globally more accessible and more reliable since it affords high sensitivity in HPV detection. However, p16 IHC lacks specificity and may be prone to false positives due in part to accumulating evidence demonstrating that a subset of HPV(−) HNSCCs overexpress p16 (39). Consequently, PCR-based assays and p16 IHC remain sub-optimal in their performance when used alone for the detection of HPV (26, 29, 39-45). The combined application of ISH to detect expression of the viral E6/E7 genes may overcome sensitivity limitations of p16 IHC but this method is prone to false negatives and is not universally available (46-52). Although IHC and ISH can be multiplexed, IHC is a relatively inexpensive and more standard assay for pathology laboratories leading many clinicians to rely on p16 IHC alone for classification of these tumors. Therefore, there is a significant unmet need to identify additional clinically useful biomarkers that suitable for multiplexed IHC on the same sample slide that provide accurate detection of HPV status to aid in stratifying patient risk better.

In addition to identifying an affordable and easily implemented HPV biomarker to complement p16 IHC, the head and neck oncology field is also in need of identifying markers that can predict response to standard and immune-based therapies as a priority. The introduction and increasing popularity of immune based therapies for various cancers, has provided a viable treatment option for some patients, but only a small proportion of HNSCC patients (15-20%) respond to these therapies (54-56). Recent studies unveiling the diversity of tumor-immune microenvironment in HNSCC present an abundance of opportunities to further examine the roles of these interactions (57-63). Importantly, HPV(+) tumors possess a unique tumor-immune landscape, including differing types, proportions, and functions of immune cells when compared to HPV(−) tumors and recent studies demonstrate these HPV(+) tumors harbor functional PD-1+TCF-1+CD45RO+ stem-like CD8 T cells suggesting that these HNSCC patients may retain the ability to respond to PD-1 checkpoint blockade (64-68).

The present invention addresses shortcomings in the art by providing methods of diagnosing and/or treating head and neck squamous cell carcinoma.

Accordingly, one aspect of the present invention provides a method of diagnosing head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a level of expression of the SYNGR3 gene and/or gene product from a sample comprising one or more immune cells obtained from a control subject (e.g., a control sample); and d) diagnosing the subject as being positive for human papillomavirus (HPV) when the level of expression of the SYNGR3 gene and/or gene product is greater than the level of expression of the SYNGR3 gene and/or gene product of the control subject.

Another aspect of the present invention provides a method of diagnosing head and neck squamous cell carcinoma (HNSCC) in a subject comprising: determining a SYNGR3 status of the subject based on a level of expression of the SYNGR3 gene and/or gene product in one or more immune cells (e.g., helper T cells) in a tumor of the subject; determining a p16 status of the subject based on a level of expression of a p16 gene and/or gene product in the tumor of the subject; and diagnosing the subject has having HPV-positive HNSCC based on the SYNGR3 status and the p16 status of the subject.

Another aspect of the present invention provides a method of diagnosing head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a reference level of expression of the SYNGR3 gene and/or gene product (e.g., a level of expression of the SYNGR3 gene and/or gene product from a reference sample and/or a reference level as established in a reference database (e.g., a patient database)); and d) diagnosing the subject as being positive for human papillomavirus (HPV) when the level of expression of the SYNGR3 gene and/or gene product is greater than the reference level of expression of the SYNGR3 gene product.

In some embodiments, methods of the present invention may further comprise treating the HNSCC in the subject with a protocol for patients with that are infected by HPV-positive HNSCC.

Accordingly, another aspect of the present invention provides a method of diagnosing and treating head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a level of expression of the SYNGR3 gene and/or gene product from a sample comprising one or more immune cells obtained from a control subject (e.g., a control sample); d) diagnosing the subject as being positive for human papillomavirus (HPV) when the level of expression of the SYNGR3 gene and/or gene product is greater than the level of expression of the SYNGR3 gene product of the control subject; and e) treating the HNSCC in the subject with a protocol for patients with that are infected by HPV-positive HNSCC.

Another aspect of the present invention provides a method of diagnosing and treating head and neck squamous cell carcinoma (HNSCC) in a subject, comprising treating the HNSCC in the subject with a protocol for patients with HPV-positive HNSCC if the subject is diagnosed as having an overexpression of SYNGR3 gene and/or gene product in one or more immune cells (e.g., T cell such as a Th1 cell) in a HNSCC tumor of the subject.

Another aspect of the present invention provides a method of diagnosing and treating head and neck squamous cell carcinoma (HNSCC) in a subject comprising: determining a SYNGR3 status of the subject based on a level of expression of the SYNGR3 gene and/or gene product in one or more immune cells (e.g., helper T cells, e.g., Th1 cells) in a tumor of the subject; determining a p16 status of the subject based on a level of expression of a p16 gene and/or gene product in the tumor of the subject; diagnosing the subject has having HPV-positive HNSCC based on the SYNGR3 status and the p16 status of the subject; and if the subject is diagnosed as having HPV-positive HNSCC, treating the HNSCC in the subject with a protocol for patients with that have HPV-positive HNSCC.

In some embodiments, the HNSCC in the subject may be treated with a protocol for patients with HPV-positive HNSCC if the subject is diagnosed as having the overexpression of SYNGR3 gene and/or gene product in the one or more immune cells (e.g., T cell such as a Th1 cell) in the HNSCC tumor and an overexpression of p16 gene and/or gene product in the HNSCC tumor.

In some embodiments, the HNSCC in the subject may be treated with a protocol for patients with HPV-positive HNSCC if the subject is further diagnosed as being HPV-positive based on HPV16 in situ hybridization (ISH).

A protocol for treating patients that have HPV-positive HNSCC may comprise any standard-of-care protocol for HNSCC and/or other HPV-positive cancers, including but not limited to administration of methotrexate, immunotherapy and/or antibody therapy (e.g., anti-checkpoint blockade antibodies (anti-PD1, anti-CTLA4, anti-PDL1, anti-PDL2, and the like, e.g., nivolumab, pembrolizumab, and the like), anti-EGFR, anti-HER2 e.g., cetuximab), chemotherapy (e.g., Larotrectinib), surgical intervention (e.g., laryngectomy, pharyngectomy, excision/dissection (e.g., tumor removal, e.g., lymph node removal)), laser therapy, radiation therapy, and brachytherapy, in any combination. Additional non-limiting treatment protocols for HNSCC include those as described in Sacco and Cohen, 2015 J. Clin Oncol. 33:33053315 (ref 5), incorporated herein by reference it in entirety. In some embodiments, a protocol for treating patients that have HPV-positive HNSCC may comprise de-escalation of one or more of any standard-of-care protocols, such as but not limited to those described above. In some embodiments, a protocol for treating patients that have HPV-positive HNSCC such as but not limited to those described above may be administered prophylactically, therapeutically, and/or palliatively.

In some embodiments, methods of the present invention may further comprise determining an ISH status of the subject by performing HPV16 in situ hybridization (ISH) and diagnosing the subject has having HPV-positive HNSCC based on the SYNGR3 status, the p16 status of the subject, and the ISH status of the subject.

In some embodiments, the SYNGR3 status may be determined based on a level of overexpression of the SYNGR3 gene and/or gene product.

In some embodiments, the level of overexpression of the SYNGR3 gene and/or gene product may be determined by quantifying the SYNGR3 gene and/or gene product expression level (e.g., the expression level of SYNGR3 gene and/or gene product detected on one or more tissue microarray (TMA) comprising the one or more immune cells of the subject and/or the control subject (e.g., the sample of the subject and/or the control subject)), and comparing the gene and/or gene product expression level quantified for the subject and quantified for the control subject. In some embodiments, the level of overexpression of the SYNGR3 gene and/or gene product may be determined by quantifying the SYNGR3 gene and/or gene product expression level (e.g., the expression level of SYNGR3 gene and/or gene product detected on one or more tissue microarray (TMA) comprising the one or more immune cells of the subject and/or the control subject (e.g., the sample of the subject and/or the control subject)), comparing the gene and/or gene product expression level quantified for the subject and quantified for the control subject, and optionally, generating a ROC curve.

In some embodiments, the level of overexpression of the SYNGR3 gene and/or gene product may be determined by quantifying the SYNGR3 gene and/or gene product expression level (e.g., the expression level of SYNGR3 gene and/or gene product detected on one or more tissue microarray (TMA) comprising the one or more immune cells of the subject (e.g., the sample of the subject)), and comparing the gene and/or gene product expression level quantified for the subject and the reference level of expression of the p16 gene and/or gene product (e.g., from a reference sample and/or a reference level as established in a reference database (e.g., a patient database)). In some embodiments, the level of overexpression of the SYNGR3 gene and/or gene product may be determined by quantifying the SYNGR3 gene and/or gene product expression level (e.g., the expression level of SYNGR3 gene and/or gene product detected on one or more tissue microarray (TMA) comprising the one or more immune cells of the subject (e.g., the sample of the subject)), comparing the gene and/or gene product expression level quantified for the subject and the reference level of expression of the p16 gene and/or gene product (e.g., from a reference sample and/or a reference level as established in a reference database (e.g., a patient database)), and optionally, generating a ROC curve.

Another aspect of the present invention provides a method of diagnosing and treating head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a reference level of expression of the SYNGR3 gene and/or gene product (e.g., a level of expression of the SYNGR3 gene and/or gene product from a reference sample and/or a reference level as established in a reference database (e.g., a patient database); d) diagnosing the subject as being positive for human papillomavirus (HPV) when the level of expression of the SYNGR3 gene and/or gene product is greater than the reference level of expression of the SYNGR3 gene and/or gene product; and e) treating the HNSCC in the subject with a protocol for patients with that are infected by HPV-positive HNSCC.

In some embodiments, methods of the present invention may further comprise the steps of: detecting a level of expression of a p16 gene and/or gene product in the sample, and comparing a level of p16 gene and/or gene product in the sample with the reference level of expression of a p16 gene and/or gene product (e.g., the level of expression of the p16 gene and/or gene product from a reference sample and/or a reference level as established in a reference database (e.g., a patient database)); wherein the subject is diagnosed as positive for HPV when the level of expression of the SYNGR3 gene and/or gene product is greater than the reference level of expression of the SYNGR3 gene and/or gene product and the level of the p16 gene and/or gene product is greater than the reference level of expression of the p16 gene and/or gene product.

In some embodiments, the methods of the present invention may further comprise the steps of: detecting a level of expression of a p16 gene and/or gene product in the sample, and comparing a level of p16 gene and/or gene product in the sample with the level of expression of a p16 gene and/or gene product from the control subject (e.g., the level of expression of a p16 gene and/or gene product of the control sample); wherein the subject is diagnosed as positive for HPV when the level of expression of the SYNGR3 gene and/or gene product is greater than the level of expression of the SYNGR3 gene and/or gene product of the control subject and the level of the p16 gene and/or gene product is greater than the level of expression of the p16 gene and/or gene product of the control subject.

In some embodiments, the detecting step may comprise performing in situ hybridization (ISH) and/or immunohistochemistry (IHC) of SYNGR3 and/or p16 gene and/or gene product on the sample of the subject and/or the control subject, for example, on one or more tissue microarray (TMA) comprising the one or more immune cells of the subject and/or the control subject.

In some embodiments, the comparing step may comprise quantifying the gene and/or gene product expression level (e.g., quantifying the expression level of SYNGR3 and/or p16 gene and/or gene product detected for example, on one or more tissue microarray (TMA) comprising the one or more immune cells of the subject and/or the control subject, e.g., the sample of the subject and/or the control subject), and comparing the quantified gene and/or gene product expression level between that of the subject and that of the control subject. In some embodiments, the comparing step may comprise quantifying the gene and/or gene product expression level (e.g., quantifying the expression level of SYNGR3 and/or p16 gene and/or gene product detected for example, on one or more tissue microarray (TMA) comprising the one or more immune cells of the subject and/or the control subject, e.g., the sample of the subject and/or the control subject), comparing the quantified gene and/or gene product expression level between that of the subject and that of the control subject, and optionally, generating a ROC curve.

In some embodiments, methods of the present invention may further comprise determining an HPV status for the subject, for example, via ISH such as HPV16 ISH.

In some embodiments, the control subject may be a sample from a control subject, e.g., a control sample. In some embodiments, the sample is from a HNSCC tumor (e.g., tumor stroma) in the subject.

In some embodiments, protein biomarkers can be detected and/or quantified using technologies well known to those of skill in the art such as gel electrophoresis, immunohistochemistry, and antibody binding. Methods for generating antibodies to a polypeptide (e.g., a SYNGR3 peptide or polypeptide or a p16 peptide or polypeptide) are well known to those of ordinary skill in the art. An antibody against a protein biomarker of the presently disclosed subject matter can be any monoclonal or polyclonal antibody, so long as it suitably recognizes the protein biomarker. In some embodiments, antibodies are produced using the protein biomarker as the immunogen according to any conventional antibody or antiserum preparation process. The presently disclosed subject matter provides for the use of both monoclonal and polyclonal antibodies. In addition, a protein used herein as the immunogen is not limited to any particular type of immunogen. For example, fragments of the protein biomarkers of the presently disclosed subject matter can be used as immunogens. The fragments can be obtained by any method including, but not limited to, expressing a fragment of the gene encoding the protein, enzymatic processing of the protein, chemical synthesis, and the like. Antibodies against the instantly disclosed biomarkers can also be purchased from commercial suppliers such as, but not limited to Santa Cruz Biotechnology, Inc. (Santa Cruz, Calif., United States of America), ABCAM® (Cambridge, Mass., United States of America), Cell Signaling Technology, Inc. (Danvers, Mass., United States of America), Thermo Fisher Scientific Inc. (Rockford, Ill., United States of America), eBioscience, Inc. (San Diego, Calif., United States of America), etc.

The antibodies of the presently disclosed subject matter can be useful for detecting and/or quantifying the protein biomarkers. For example, antibody binding can be detected by techniques known in the art (e.g., radioimmunoassay, ELISA (enzyme-linked immunosorbent assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, flow cytometry, and immunoelectrophoresis assays, etc. One example of an immunoassay is described in U.S. Pat. Nos. 5,599,677 and 5,672,480, the disclosure of each of which is herein incorporated by reference. Upon review of the present disclosure, those skilled in the art will be familiar with numerous specific immunoassay formats and variations thereof that can be useful for carrying out the methods of the presently disclosed subject matter.

Approaches for producing a detectable signal include the use of radioactive labels (e.g., ³²P, ³⁵S, ¹²⁵I, ¹³¹I, and the like), enzyme labels (e.g., horseradish peroxidase, alkaline phosphatase), fluorescent labels (e.g., fluorescein, rhodamine, and fluorophores of the ALEXA-FLUOR® brand series of fluorescent dye labels available from the MOLECULAR PROBES® division of Thermo Fisher Scientific Inc., Eugene, Oreg., United States of America) and so forth, in accordance with known techniques, as will be apparent to one skilled in the art upon review of the present disclosure. Many methods are known in the art for detecting binding in an immunoassay or in a nucleic acid assay and are within the scope of the presently disclosed subject matter.

In some embodiments, direct detection methods are provided, such as, for example, wherein the detection molecule is a primary antibody specific for a biomarker and detection is by using a label present on the primary antibody. In some embodiments, the detection molecule can be detected using an indirect method such as using a labeled secondary antibody that detects the presence of the primary antibody by binding to the primary antibody per se. For example, if the primary antibody is a mouse monoclonal antibody that is specific for a biomarker of the presently disclosed subject matter, a detectably labeled anti-mouse antibody (e.g., an anti-mouse IgG or IgM secondary antibody raised in a species other than mice) can be used to detect the presence of the primary antibody bound to the biomarker.

In some embodiments, the presence or absence of the biomarkers SYNGR3 and p16 are determined simultaneously. This can be accomplished in some embodiments by conjugating differently detectable labels to an anti-SYNGR3 primary antibody and an anti-p16 primary antibody. In some embodiments, this can be accomplished by using unlabeled primary antibodies that can be differentially detected using secondary antibodies that are conjugated to different detectable labels. In some embodiments, the presence or absence of the biomarkers SYNGR3 and p16 are determined sequentially, for example by detecting SYNGR3 expression in one section of a tumor and detecting p16 expression in a serial section of the same tumor. Serial sections can be assayed on separate slides or on the same slide provided that the slide contains a barrier to prevent intermixing of reagents.

The phrase “detection molecule” is used herein in its broadest sense to include any molecule that can bind with sufficient specificity to a biomarker to allow for detection of the particular biomarker. To allow for detection can mean to determine the presence or absence of the particular biomarker member and, in some embodiments, can mean to determine the amount of the particular biomarker. Detection molecules can include, but are not limited to antibodies, antibody fragments, and nucleic acid sequences. In some embodiments, the detection molecules comprise a conjugated detectable group. In some embodiments, the detection molecules comprise antibodies specific for each of the protein biomarkers.

Thus, the detection molecule can in some embodiments be detected using an indirect method such as by detecting binding of a specific binding partner to the detection molecule. The specific binding partner can be any molecule that binds with sufficient specificity to the detection molecule to allow for detection of the particular detection molecule in the presence or absence of the detection molecules for the other biomarker. In some embodiments, the detection molecule is a primary antibody and the primary antibody can be detected by detecting binding of a secondary antibody or a reagent or other specific binding partner to the primary antibody. For example, in some embodiments the specific binding partner can be a secondary antibody that recognizes the detection molecule that is a primary antibody. In some embodiments the specific binding partner can be a molecule that specifically binds to a group on the detection molecule such as, for example, a biotin group on the detection molecule. In some embodiments, the binding partner can be labeled. In some embodiments, the binding partner is a secondary antibody that can be labeled.

The phrase “a specific binding partner for each of the detection molecules” is used herein to include any molecule that binds with sufficient specificity to one of the detection molecules to allow for detection of the particular detection molecule. For example, in some embodiments the specific binding partner can be a secondary antibody that recognizes the detection molecule that is a primary antibody. In some embodiments the specific binding partner can be a molecule that specifically binds to a group on the detection molecule such as, for example, a biotin group on the detection molecule.

Indirect detection methods can in some embodiments involve a detection molecule that is an unlabeled primary antibody and a binding partner that is a labeled secondary antibody. This method can be more sensitive than direct detection methods due to signal amplification through more than one secondary antibody reaction with different antigenic sites on the primary antibody. In some embodiments, the indirect detection method is an immunofluorescence method, wherein the secondary antibody can be labeled with a fluorescent dye such as FITC, rhodamine, Texas red, or an ALEXA-FLUORO dye. In some embodiments, the indirect detection method is an immunoenzyme method, wherein the secondary antibody can be labeled with an enzyme such as peroxidase, alkaline phosphatase, or glucose oxidase.

In some embodiments, an immunoassay can comprise antibodies specific for one or more biomarkers and an approach for producing a detectable signal. In some embodiments, the antibodies can be immobilized on a support (such as a bead, plate, or slide) in accordance with known techniques, and contacted with a test sample in liquid phase. The support can then be separated from the liquid phase and either the support phase or the liquid phase can be examined for the detectable signal that is related to the presence of the biomarker.

Accordingly, in some embodiments a sample is a tissue section and the detecting step comprises immunohistochemically staining the sample with a primary antibody that binds to a SYNGR3 gene product and detecting binding of the primary antibody to the SYNGR3 gene product. In some embodiments, the primary antibody comprises a detectable label and detecting binding of the primary antibody to the SYNGR3 gene product comprises detecting the detectable label. In some embodiments, detecting binding of the primary antibody to the SYNGR3 gene product comprises detecting a complex of the primary antibody and SYNGR3 gene product using a labeled secondary antibody that is specific for the primary antibody. In some embodiments, the sample to be assayed for SYNGR3 is a cell extract and the contacting and detecting steps comprise immunoblotting with a primary antibody comprising a detectable label that is specific for the SYNGR3 gene product and detecting the detectable label; or immunoblotting with a primary antibody that is specific for the SYNGR3 gene product and detecting the primary antibody indirectly with a labeled secondary antibody that binds to the primary antibody. Detecting the p16 gene product may be performed in the same manner.

In some embodiments, the detecting step may comprise performing ISH and/or immunohistochemistry (IHC) of a gene product (SYNGR3 and/or p16) on one or more tissue microarray (TMA) comprising a sample of the subject and/or the control subject (e.g., a sample comprising one or more immune cells of the subject and/or the control subject). Upon review of the present disclosure, those skilled in the art will be familiar with numerous standard methods known in the art for analysis of microarray data, including but not limited to analysis of relative quantification and/or performing significance analysis of microarrays (SAM) analysis of gene product copy number normalized to a control gene product such as but not limited to any housekeeping gene product known in the art (e.g., a typically constitutive gene product required for maintenance of basic cellular function and/or expressed in all cells of an organism under normal and pathophysiological conditions), e.g., ribosomal gene products (e.g., RPL9, RPL 13A, RPL23, and the like), RNA polymerase gene products (e.g., POLR1C, POLR1D, POLR1E, and the like), heat shock proteins (e.g., HSPA4, HSPA5, HSPA8, and the like), histones (e.g., HIST1H2BC, H1FX, H2AFV, and the like), and other well-established housekeeping gene products such as but not limited to ACTB, GAPDH, and PP1A. Microarray methods, uses thereof, and analyses thereof are further discussed in Miller and Tang, (2009) Clin. Microbiol. Rev. 22(4):611-633; Chu et al., “SAM “Significance Analysis” of microarrays users guide and technical document,” www-stat.stanford.edu/˜tibs/SAM/sam.pdf; and Zang et al. (2007) J. of Biomedical Informatics 40(5):552-560, the disclosures of each of which are incorporated herein by reference.

In some embodiments, the comparing step mat comprise quantifying a gene product expression level (e.g., the expression level of SYNGR3 and/or p16 gene product), and comparing the quantified gene product expression level between that of the subject and that of the control subject. In some embodiments, the comparing step may further comprise generating a ROC curve.

Image-based methods known in the art for quantification of the presence and/or expression level of gene products and/or biomarkers include the image processing suites ImageJ, ImageJ2, and Fiji, as disclosed, respectively, in Schneider, et al. (2012) Nature Methods, 9(7), 671-675; Rueden, et al. (2017) BMC Bioinformatics, 18(1); and Schindelin, et al. (2012) Nature Methods, 9(7), 676-682; the disclosures of each of which is herein incorporated by reference. A further non-limiting example of an image-based method known in the art includes Aperio Digital Pathology slide scanner and related software available from the Leica Biosystems Inc., Buffalo Grove, Ill., United States of America.

Upon review of the present disclosure, those skilled in the art will be familiar with numerous standard methods known in the art for analyzing sensitivity and/or specificity of biomarkers. In some embodiments, the results of biomarker expression data may be expressed in terms of receiver operating characteristic (ROC) curves. ROC curves may be generated from biomarker expression data to illustrate the diagnostic ability of a binary classifier system (e.g., “positive” or “negative”) by plotting the true positive rate (TPR; also referred to as sensitivity or probability of detection) against the false positive rate (FPR; also referred to as specificity or probability of false alarm) at various determined threshold settings. ROC curves and related non-limiting statistical models applicable in the present invention are broadly discussed in Fawcett, T. (2006) Pattern Recognition Letters. 27(8): 861-874; and Tharwat A., (2021) Applied Computing and Informatics (17)1:168-192.

In some embodiments, the results of the various antibody-based assays may be expressed in terms of a “histochemistry score”, also known as an HSCORE. HSCOREs are expressions of antibody staining intensity, and are broadly discussed in Lessey et al., 1992. By way of example and not limitation, in some embodiments an HSCORE is calculated using the following equation:

HSCORE=ΣPi(i+1)/100

where i=the intensity of staining of cells in the sample with a value of 1 being low staining, 2 being moderate staining, and 3 being strong staining, and Pi being the percentage of stained cells in the sample for each intensity, varying from 0-100%. An HSCORE can function as a pre-determined cut-off such that expression above or below a pre-determined HSCORE in a particular subject for a particular biomarker can permit that subject's status for that biomarker to be identified as “normal” vs. “abnormal”, positive vs. negative, or any other discriminator. With respect to SYNGR3, for example, in some embodiments an abnormal SYNGR3 status may comprise an HSCORE for the subject with respect to SYNGR3 gene product expression that is greater than a pre-determined cut-off value, which in some embodiments can be selected from the group consisting of 1.0, 1.1, 1.2, 1.3, 1.4, 1.5, 1.6, 1.7, 1.8, 1.9, and 2.0. The HSCORE system may be used for p16 in the same manner.

As used herein, “treating HNSCC” refers to any treatment known for treating HNSCC including but not limited to resection, radiation, and/or systemic therapy. HPV-positive HNSCC patients generally have higher survival rates than HPV negative patients, and so if a HNSCC patient is identified as being HPV-positive, the patient may be treated with a less aggressive or toxic modality. For example, in some embodiments, an HPV positive patient may be treated by surgery only while an HPV-negative patient may have surgery and systemic therapy.

As such, in some embodiments the presently disclosed subject matter provides methods for detecting the presence of HPV in a subject having HNSCC by assaying for the presence or absence of the SYNGR3, optionally in combination with other biomarkers such as p16, in an immune cell (e.g., T cell) of a subject. In some embodiments, the presently disclosed methods comprise (a) providing a subject with HNSCC; (b) detecting the presence or absence of biomarker SYNGR3 and optionally biomarker p16, in immune cells in a sample (e.g., tumor sample) from the subject; and (c) determining the presence of HPV in the subject based on the detecting in step (b).

In some embodiments of the presently disclosed subject matter, a kit is provided for measuring the presence and/or amount of one or more biomarkers in a sample of the subject. In some embodiments, the kit can comprise (i) detection molecules specific for a biomarker such as SYNGR3, and optionally p16; and (ii) directions for measuring the presence or amount of a biomarker. In some embodiments, the kit can also include directions for using the determined biomarker levels in managing treatment.

In some embodiments, the present invention may be as defined in any one of the following numbered paragraphs.

1. A method of diagnosing head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a level of expression of the SYNGR3 gene and/or gene product from a sample comprising one or more immune cells obtained from a control subject (e.g., a control sample); and d) diagnosing the subject as being positive for human papillomavirus (HPV) when the level of expression of the SYNGR3 gene and/or gene product is greater than the level of expression of the SYNGR3 gene and/or gene product of the control subject.
2. The method of paragraph 1, further comprising treating the HNSCC in the subject with a protocol for patients with that are infected by HPV-positive HNSCC.
3. A method of diagnosing and treating head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a level of expression of the SYNGR3 gene and/or gene product from a sample comprising one or more immune cells obtained from a control subject (e.g., a control sample); d) diagnosing the subject as being positive for human papillomavirus (HPV) when the level of expression of the SYNGR3 gene and/or gene product is greater than the level of expression of the SYNGR3 gene product of the control subject; and e) treating the HNSCC in the subject with a protocol for patients with that are infected by HPV-positive HNSCC.
4. The method of any one of paragraphs 1-3, wherein the sample is from a HNSCC tumor (e.g., tumor stroma) in the subject.
5. The method of any one of paragraphs 1-4, wherein the gene product comprises one or more of a mRNA and a protein.
6. The method of paragraph 5, wherein the gene product comprises mRNA and a protein.
7. The method of any of paragraphs 1-6, further comprising the steps of: detecting a level of expression of a p16 gene and/or gene product in the sample, and comparing a level of p16 gene and/or gene product in the sample with the level of expression of a p16 gene and/or gene product from the control subject (e.g., the level of expression of a p16 gene and/or gene product of the control sample); wherein the subject is diagnosed as positive for HPV when the level of expression of the SYNGR3 gene and/or gene product is greater than the level of expression of the SYNGR3 gene and/or gene product of the control subject and the level of the p16 gene and/or gene product is greater than the level of expression of the p16 gene and/or gene product of the control subject.
8. The method of any one of paragraphs 1-7, wherein the detecting step comprises performing in situ hybridization (ISH) and/or immunohistochemistry (IHC) of SYNGR3 and/or p16 gene and/or gene product on the sample of the subject and/or the control subject (e.g., on one or more tissue microarray (TMA) comprising the one or more immune cells of the subject and/or the control subject).
9. The method of any one of paragraphs 1-8, wherein the comparing step comprises quantifying the gene and/or gene product expression level (e.g., the expression level of SYNGR3 and/or p16 gene and/or gene product detected on one or more tissue microarray (TMA) comprising the one or more immune cells of the subject and/or the control subject (e.g., the sample of the subject and/or the control subject)), comparing the quantified gene and/or gene product expression level between that of the subject and that of the control subject, and optionally, generating a ROC curve.
10. The method of paragraphs 1-9, further comprising determining an HPV status for the subject via HPV16 (e.g., via ISH).
11. A method of diagnosing and treating head and neck squamous cell carcinoma (HNSCC) in a subject, comprising treating the HNSCC in the subject with a protocol for patients with HPV-positive HNSCC if the subject is diagnosed as having an overexpression of SYNGR3 gene and/or gene product in one or more immune cells (e.g., T cell such as Th1 cell) in a HNSCC tumor of the subject.
12. The method of paragraph 11, wherein the HNSCC in the subject is treated with a protocol for patients with HPV-positive HNSCC if the subject is diagnosed as having the overexpression of SYNGR3 gene and/or gene product in the one or more immune cells (e.g., T cell such as Th1 cell) in the HNSCC tumor and an overexpression of p16 gene and/or gene product in the HNSCC tumor.
13. The method of paragraph 11 or 12, wherein the HNSCC in the subject is treated with a protocol for patients with HPV-positive HNSCC if the subject is further diagnosed as being HPV-positive based on HPV16 in situ hybridization (ISH).
14. A method of diagnosing head and neck squamous cell carcinoma (HNSCC) in a subject comprising: determining a SYNGR3 status of the subject based on a level of expression of the SYNGR3 gene and/or gene product in one or more immune cells (e.g., helper T cells) in a tumor of the subject; determining a p16 status of the subject based on a level of expression of a p16 gene and/or gene product in the tumor of the subject; and diagnosing the subject has having HPV-positive HNSCC based on the SYNGR3 status and the p16 status of the subject.
15. The method of paragraph 14, further comprising treating the HNSCC in the subject with a protocol for patients with that have HPV-positive HNSCC.
16. A method of diagnosing and treating head and neck squamous cell carcinoma (HNSCC) in a subject comprising: determining a SYNGR3 status of the subject based on a level of expression of the SYNGR3 gene and/or gene product in one or more immune cells (e.g., helper T cells) in a tumor of the subject; determining a p16 status of the subject based on a level of expression of a p16 gene and/or gene product in the tumor of the subject; diagnosing the subject has having HPV-positive HNSCC based on the SYNGR3 status and the p16 status of the subject; and if the subject is diagnosed as having HPV-positive HNSCC, treating the HNSCC in the subject with a protocol for patients with that have HPV-positive HNSCC.
17. The method of any one of paragraphs 14-16, further comprising determining an ISH status of the subject by performing HPV16 in situ hybridization (ISH) and diagnosing the subject has having HPV-positive HNSCC based on the SYNGR3 status, the p16 status of the subject, and the ISH status of the subject.
18. The method of any one of paragraphs 14-17, wherein the SYNGR3 status is determined based on a level of overexpression of the SYNGR3 gene and/or gene product.
19. The method of paragraph 18, wherein the level of overexpression of the SYNGR3 gene and/or gene product is determined by quantifying the SYNGR3 gene and/or gene product expression level (e.g., the expression level of SYNGR3 gene and/or gene product detected on one or more tissue microarray (TMA) comprising the one or more immune cells of the subject and/or the control subject (e.g., the sample of the subject and/or the control subject)), comparing the gene and/or gene product expression level quantified for the subject and quantified for the control subject, and optionally, generating a ROC curve.
20. The method of paragraph 18, wherein the level of overexpression of the SYNGR3 gene and/or gene product is determined by quantifying the SYNGR3 gene and/or gene product expression level (e.g., the expression level of SYNGR3 gene and/or gene product detected on one or more tissue microarray (TMA) comprising the one or more immune cells of the subject (e.g., the sample of the subject)), comparing the gene and/or gene product expression level quantified for the subject and the reference level of expression of the p16 gene and/or gene product (e.g., from a reference sample and/or a reference level as established in a reference database (e.g., a patient database)), and optionally, generating a ROC curve.
21. A method of diagnosing head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a reference level of expression of the SYNGR3 gene and/or gene product (e.g., a level of expression of the SYNGR3 gene and/or gene product from a reference sample and/or a reference level as established in a reference database (e.g., a patient database)); and d) diagnosing the subject as being positive for human papillomavirus (HPV) when the level of expression of the SYNGR3 gene and/or gene product is greater than the reference level of expression of the SYNGR3 gene product.
22. The method of paragraph 21, further comprising treating the HNSCC in the subject with a protocol for patients with that have HPV-positive HNSCC.
23. A method of diagnosing and treating head and neck squamous cell carcinoma (HNSCC) in a subject, comprising: a) obtaining a sample from the subject comprising one or more immune cells; b) detecting a level of expression of a SYNGR3 gene and/or gene product in the one or more immune cells; c) comparing the level of expression detected in (b) with a reference level of expression of the SYNGR3 gene and/or gene product (e.g., a level of expression of the SYNGR3 gene and/or gene product from a reference sample and/or a reference level as established in a reference database (e.g., a patient database); d) diagnosing the subject as being positive for human papillomavirus (HPV) when the level of expression of the SYNGR3 gene and/or gene product is greater than the reference level of expression of the SYNGR3 gene and/or gene product; and e) treating the HNSCC in the subject with a protocol for patients with that are infected by HPV-positive HNSCC.
24. The method of any one of paragraphs 21-23, wherein the sample is from a HNSCC tumor (e.g., tumor stroma) in the subject.
25. The method of any one of paragraphs 21-24, wherein the gene product comprises one or more of a mRNA and a protein.
26. The method of paragraph 25, wherein the gene product comprises mRNA and a protein.
27. The method of any of paragraphs 21-26, further comprising the steps of: detecting a level of expression of a p16 gene and/or gene product in the sample, and comparing a level of p16 gene and/or gene product in the sample with the reference level of expression of a p16 gene and/or gene product (e.g., the level of expression of the p16 gene and/or gene product from a reference sample and/or a reference level as established in a reference database (e.g., a patient database)); wherein the subject is diagnosed as positive for HPV when the level of expression of the SYNGR3 gene and/or gene product is greater than the reference level of expression of the SYNGR3 gene and/or gene product and the level of the p16 gene and/or gene product is greater than the reference level of expression of the p16 gene and/or gene product.
28. The method of any one of paragraphs 21-27, wherein the detecting step comprises performing in situ hybridization (ISH) and/or immunohistochemistry (IHC) of SYNGR3 and/or p16 gene and/or gene product on the sample of the subject (e.g., on one or more tissue microarray (TMA) comprising the one or more immune cells of the subject).
29. The method of any one of paragraphs 21-28, wherein the comparing step comprises quantifying the gene and/or gene product expression level (e.g., the expression level of SYNGR3 and/or p16 gene and/or gene product detected on one or more tissue microarray (TMA) comprising the one or more immune cells of the subject (e.g., the sample of the subject)), comparing the quantified gene and/or gene product expression level between that of the subject and reference level of expression of p16 gene and/or gene product, and optionally, generating a ROC curve.
30. The method of paragraphs 21-28, further comprising determining an HPV status for the subject via HPV16 (e.g., via ISH).

The present invention is explained in greater detail in the following non-limiting examples.

### EXAMPLES

### Example 1

Bioinformatics analyses of The Cancer Genome Atlas (TCGA) Head and Neck Squamous Cell Carcinoma (HNSCC) dataset was performed. Using an immunogenomics approach, patterns of differential gene expression unique to HPV negative versus HPV positive HNSCC cases were identified. Synaptogyrin 3 (SYNGR3) was identified as a top unregulated gene in the HPV positive subset. Notably, analysis of immune cell gene signatures revealed that SYNGR3 expression corresponds to the T helper type 1 (Th1) cells which are a lineage of CD4+ effector T cells that promote cell-mediated immune responses required for host defense against intracellular viral and bacterial pathogens.

These bioinformatics results were confirmed by both qPCR for SYNGR3 and immunohistochemistry (IHC) of SYNGR3 using a cohort of 11 fresh frozen HNSCC tumors whose HPV status was tested by conventional p16 IHC and/or HPV in situ hybridization (ISH). The specificity of SYNGR3 for HPV positive tumors was further validated by performing IHC on a larger cohort of 176 tumors from an available CHANCE tumor tissue microarray where HPV status was tested by both p16 IHC and HPV in situ hybridization (ISH). SYNGR3 expression was confirmed to be specific to T cells in HPV positive HNSCCs by performing multiplex staining for a pan T cell marker (CD3) and SYNGR3 quantifying expression in an unbiased manner using the Aperio digital pathology slide scanner. The results of this quantification were then used to generate receiver operator curves (ROC) comparing p16 alone, SYNGR3 alone, and p16+SYNGR3 and indicate that SYNGR3 significantly increase the sensitivity and specificity of detecting a TRUE HPV positive HNSCC case. Lastly, these findings were also verified using an independent antibody to SYNGR3 purchased from a different vendor.

Objectives: Nearly 60% of oropharyngeal head and neck squamous cell carcinomas (HNSCCs) are positive for the human papillomavirus-16 (HPV16). While HPV is a prognostic factor of these cancers, interpretation of available HPV detection assays remains challenging. Both p16 IHC and HPV16 ISH are currently used detection methods but suffer from sub-optimal specificity and sensitivity, respectively. We employed an immunogenomics approach to identify and validate a complementary method of establishing HPV status in HNSCCs.

Methods: To assess tumor-immune interactions, we surveyed available TCGA data and identified genes differentially expressed (DEGs) in immune cells between HPV(+) and HPV(−) HNSCCs. Candidate genes were tested in clinical specimens using both quantitative RT-PCR and immunohistochemistry (IHC), and then validated by IHC of a tissue microarray (TMA) of HNSCC cases. Multiplex immunofluorescent staining of the same TMA was then performed with immune cell markers to confirm expression in the immune compartment of the tumor. Lastly, Receiver Operating Characteristic (ROC) curve analysis was performed to assess the sensitivity and specificity relative to existing HPV detection assays.

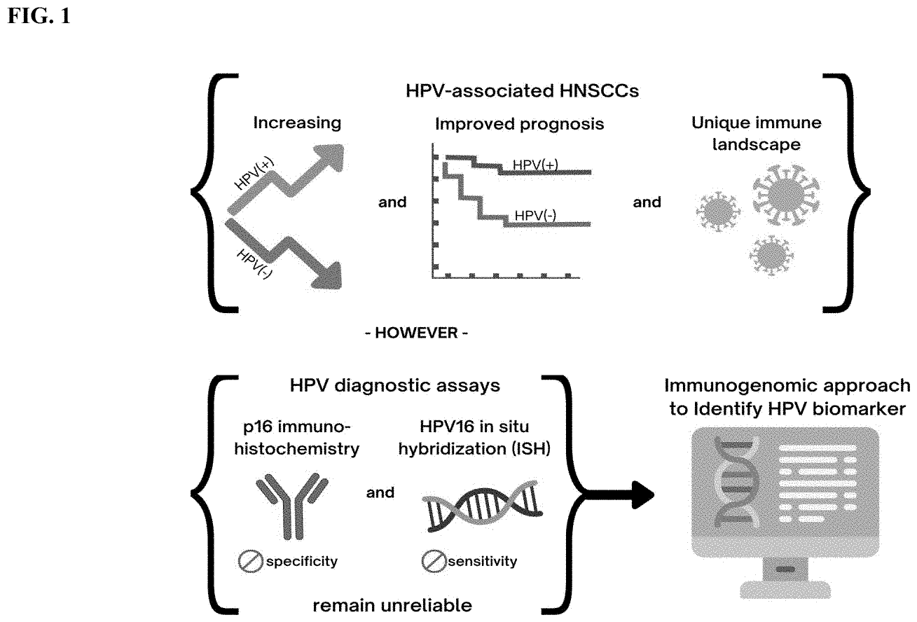

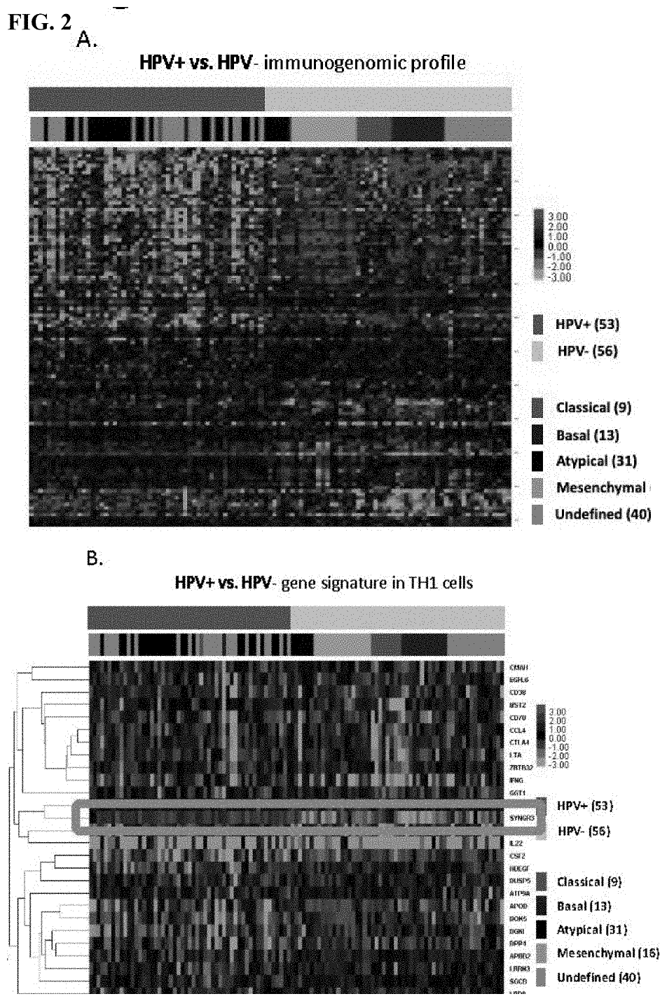

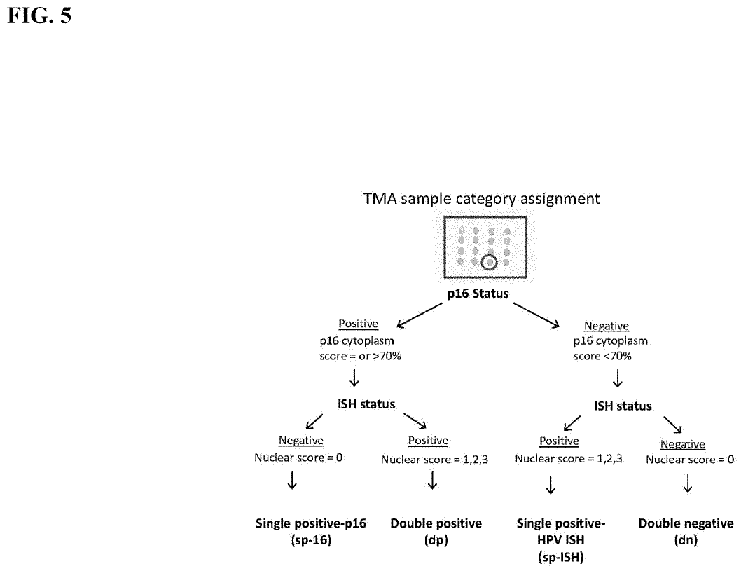

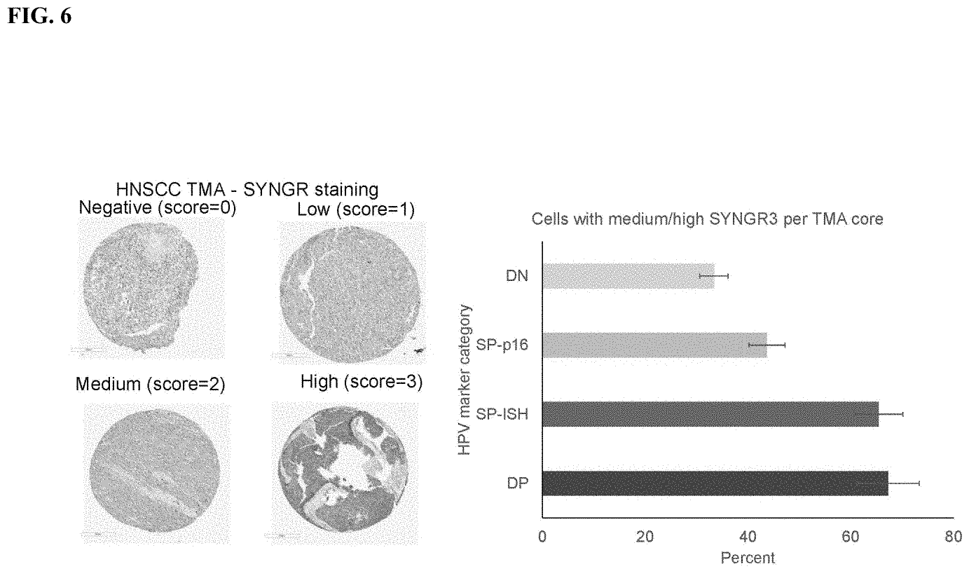

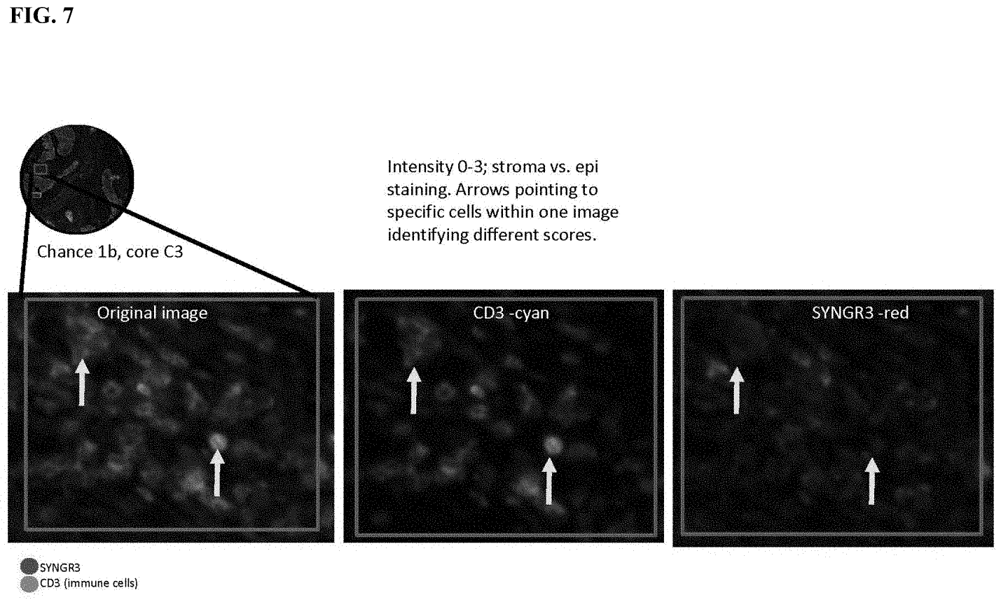

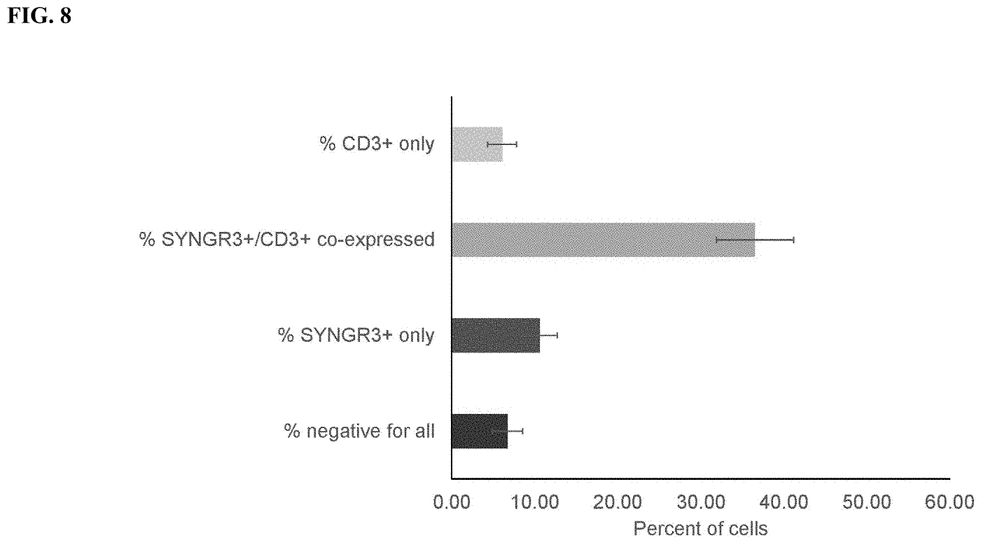

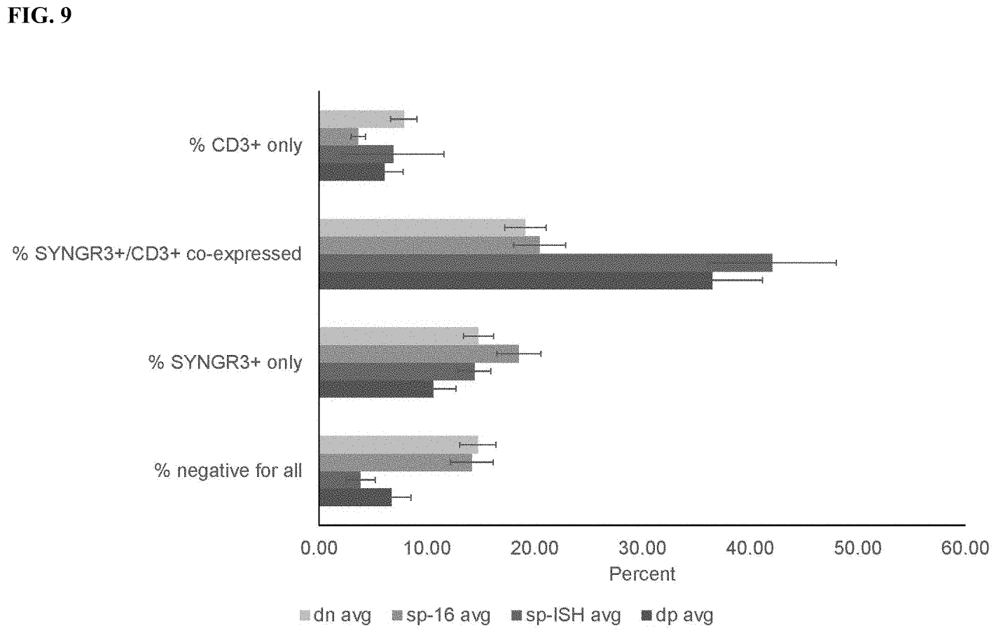

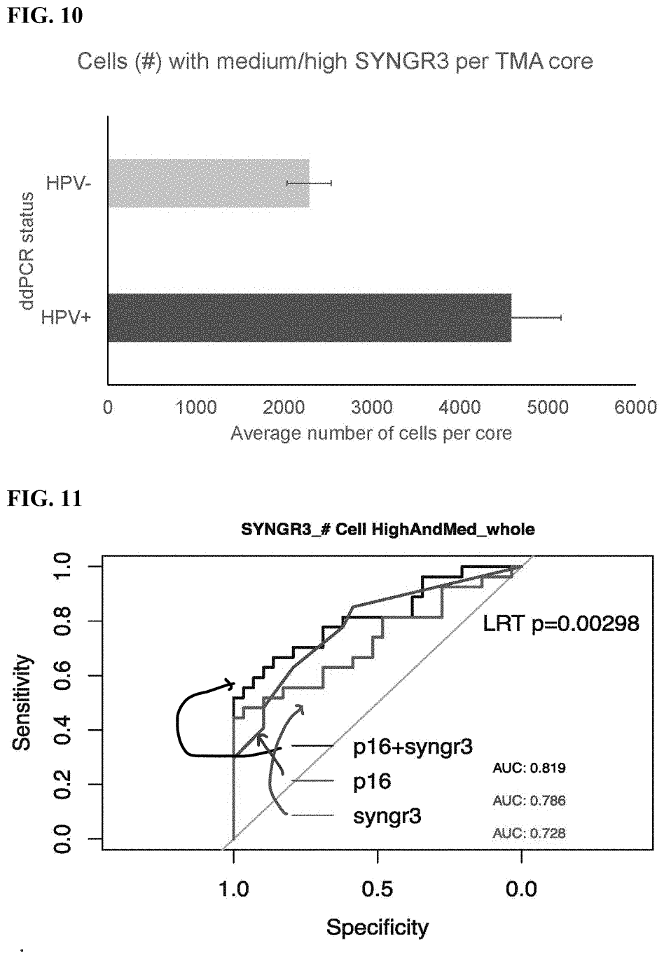

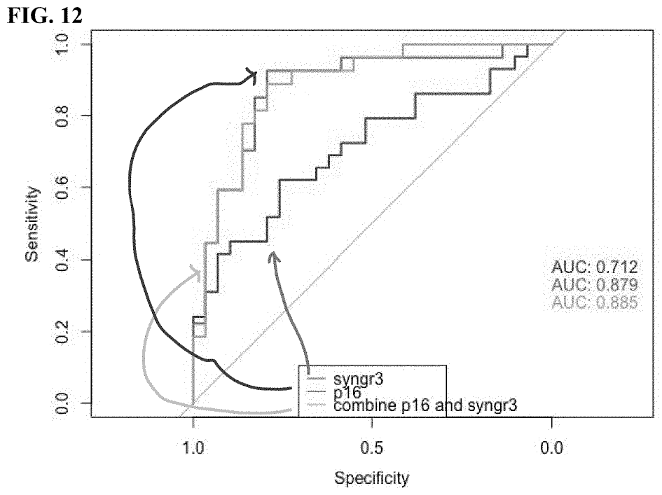

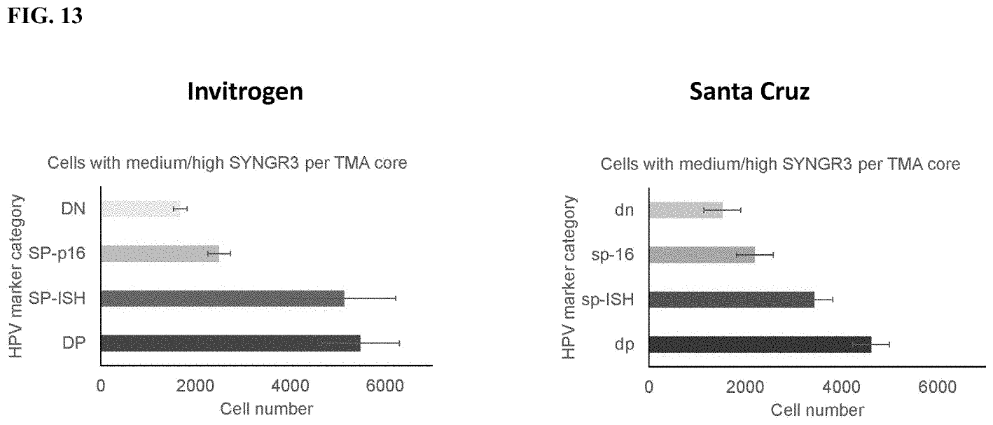

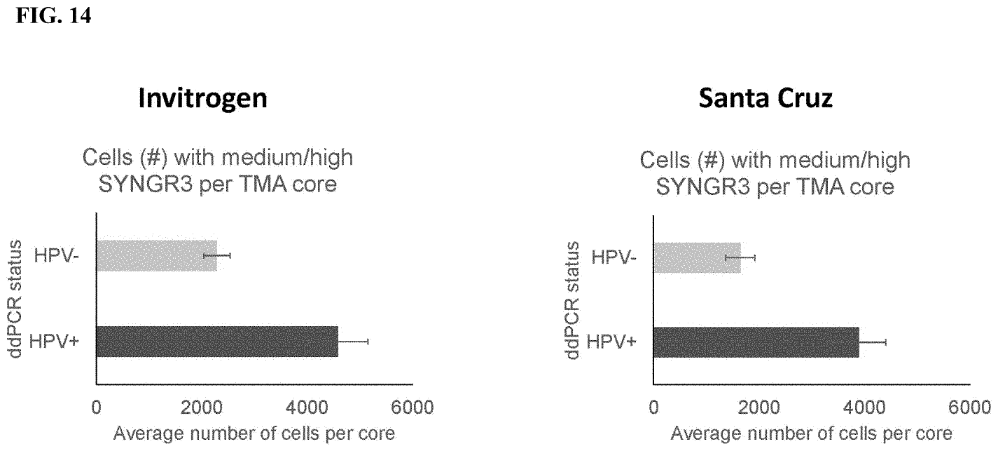

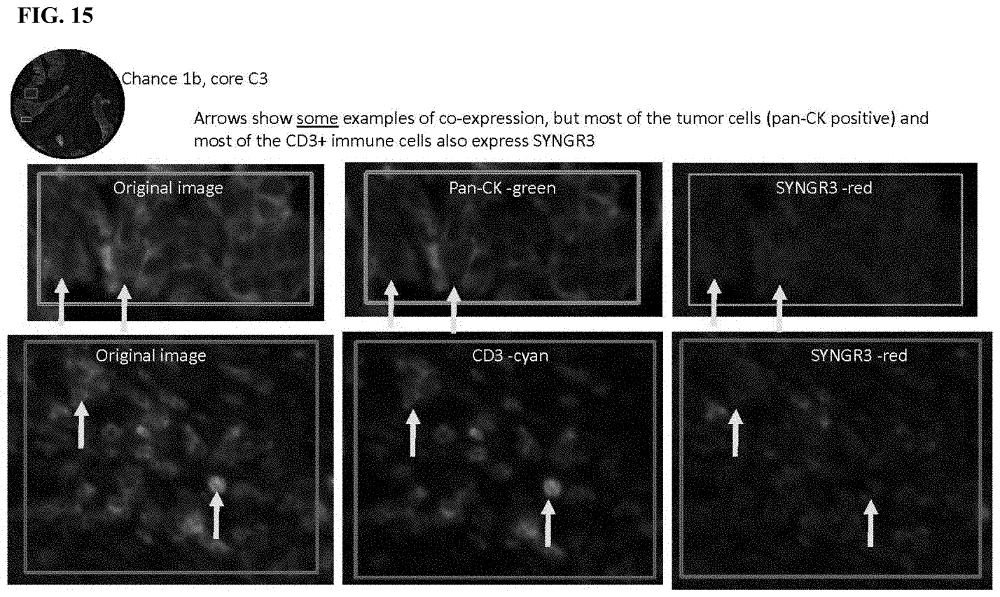

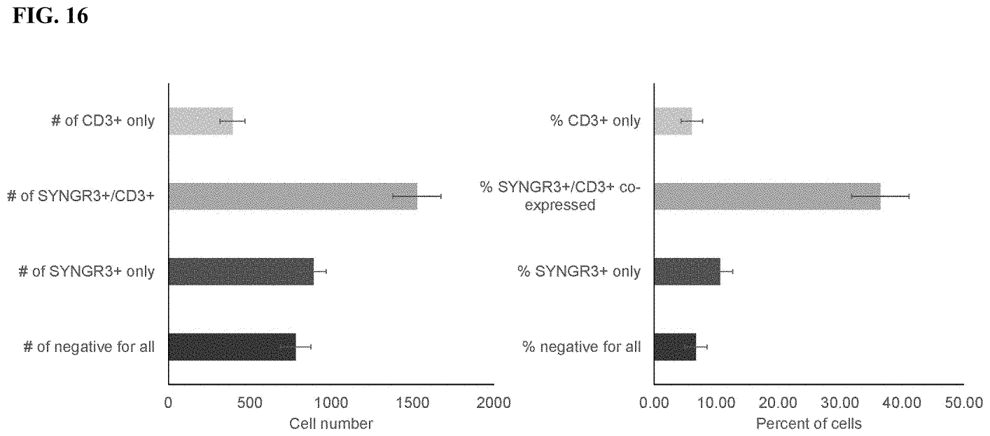

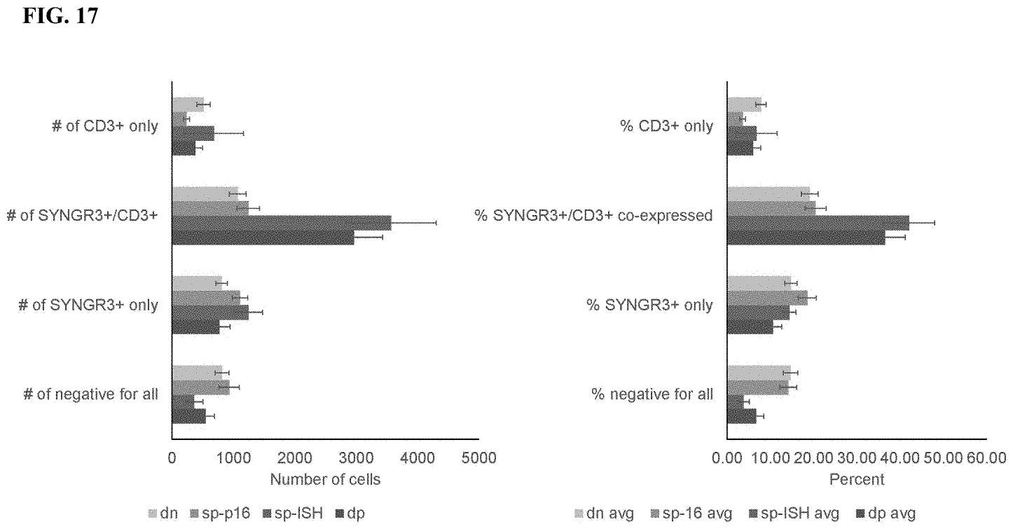

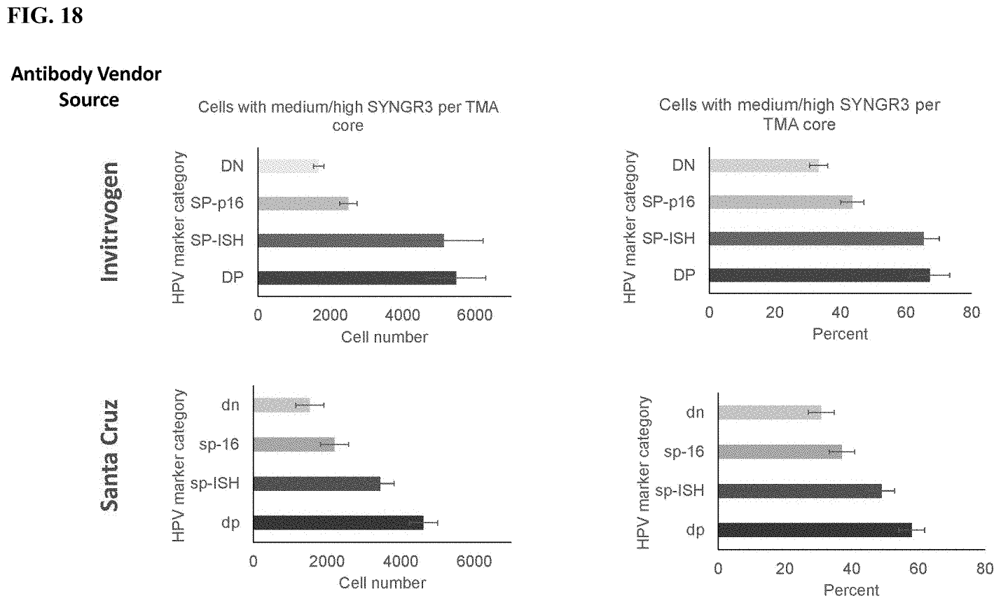

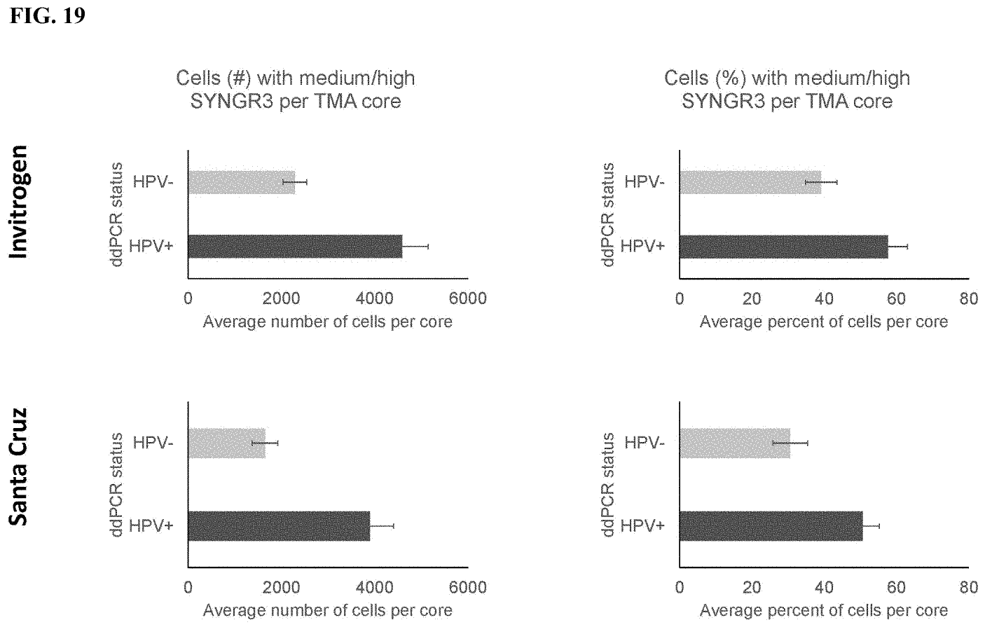

Results: HPV(+) HNSCCs have a unique immunogenomic signature that includes robust expression of Synaptogyrin-3 (SYNGR3) in Th1 cells within the tumor stroma. SYNGR3 mRNA and protein are significantly higher in primary HPV(+) HNSCC patient tumors relative to their HPV(−) counterpart, and pan-CD3/SYNGR3/pan-Cytokeratin multiplex staining successfully confirmed that SYNGR3 is enriched in T cells of these tumors. ROC curve analyses revealed that co-detection of SYNGR3 and p16 provides more sensitivity and specificity for HPV detection when compared to p16 IHC alone. Data from these studies are represented in FIGS. 1-19 and Table 1.

Conclusion: HPV(+) HNSCCs harbor T cells that exhibit robust SYNGR3 expression supporting its use as a novel biomarker of HPV positivity. These data indicate that co-detection of SYNGR3 and p16 by IHC can more reliably predict HPV status in HNSCCs, and therefore has diagnostic and prognostic value in the clinic.

### Example 2

Experiments and data described in Example 1 were further analyzed as described herein.

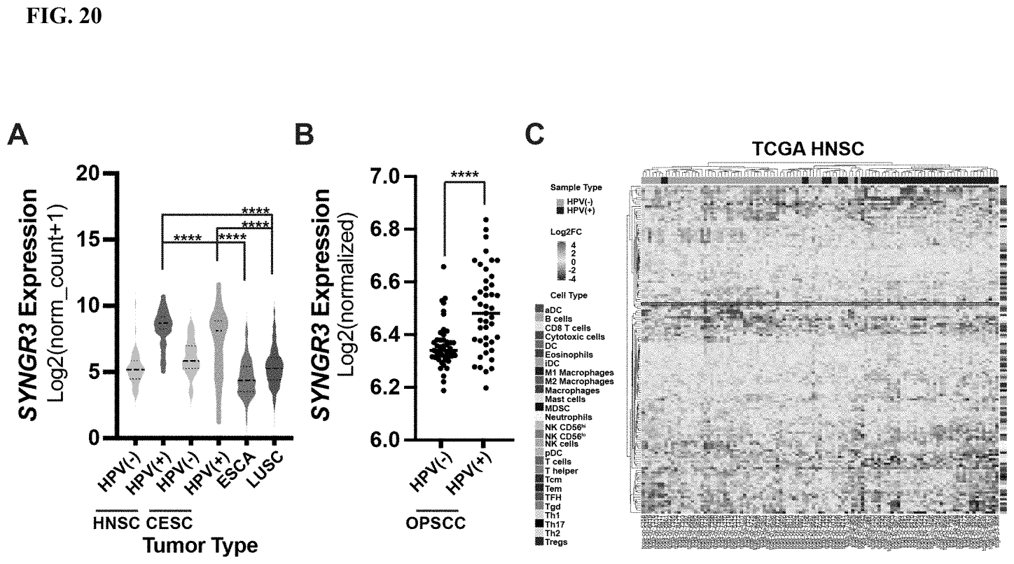

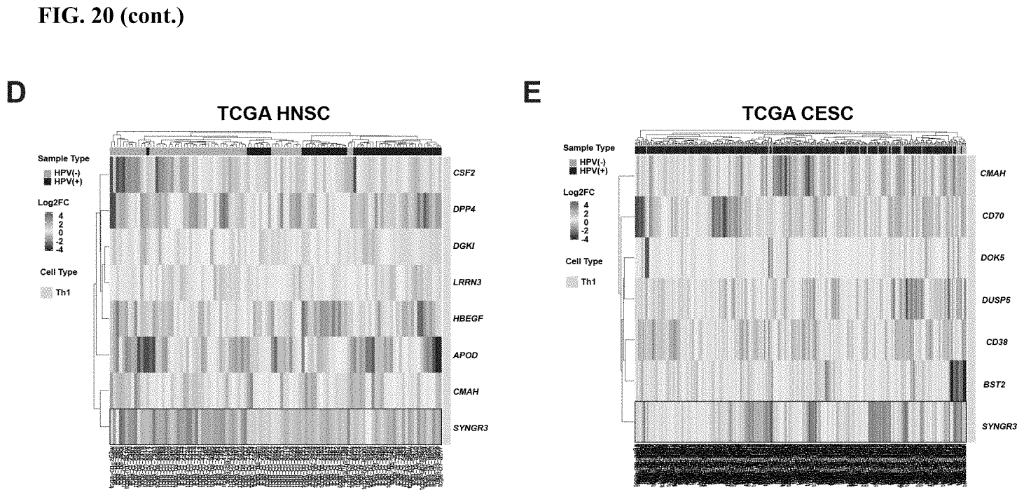

Identification of SYNGR3 within immune cells of HPV(+) HNSCCs: To identify a biomarker of HPV positive head and neck cancers, it was first determined which genes are differentially expressed between HPV(+) and HPV(−) tumors using the TCGA HNSCC dataset. Specifically, gene expression in tumors for which clinical information was available were examined. Out of the original 279 published HNSCC tumors analyzed (9), samples were categorized based on p16 IHC and HPV16 E6/E7 ISH status which resulted in 53 HPV(+) samples (positive for both p16 IHC and HPV16 E6/E7 ISH) and 56 HPV(−) samples (negative for both p16 IHC and HPV16 E6/E7 ISH). Unsupervised hierarchical clustering of these data confirmed previous studies (7, 83-85) demonstrating that HPV(+) and HPV(−) tumors have distinct transcriptomic profiles. SYNGR3 was fourth most significantly (p=9.40E-79) upregulated gene identified in HPV(+) tumors. Notably, analysis of cervical squamous cell carcinomas (CESC), which are known to be predominantly driven by HPV, revealed that SYNGR3 is also differentially expressed in these tumors based on HPV status (p=2.75E-07). To further test the association between SYNGR3 expression and HPV status, all available TCGA PanCancer squamous cell carcinoma datasets were analyzed and it was confirmed that expression of SYNGR3 is significantly elevated only in HPV(+) tumors (FIG. 20 panel A). Moreover, additional analyses confirm that SYNGR3 expression is high in HPV(+) oropharyngeal SCC (OPSCC) and low in HPV(−) OPSCC (FIG. 20 panel B), suggesting that SYNGR3 is indeed a diagnostic and prognostic biomarker of head and neck cancers.

Despite recent progress that has identified a role for SYNGR3 in regulating synaptic vesicles and neuronal function (70, 86-88), a significant knowledge gap remains in our understanding of SYNGR3 biology. Two closely related family members, SYNGR1 and SYNGR2, were shown to be expressed within immune cells (89, 90). Given this potential neuronal-independent role for SYNGR3 in immunobiology and the unique tumor-immune landscape that exists between HPV(+) and HPV(−) HNSCCs (68), immune-related gene expression profiles were examined. A ‘Modified Immunome Signature’ based on curated immunogenomics signatures (60, 62, 73) was applied and unsupervised hierarchical clustering of DEGS in HPV(+) HNSCC and (HPV+) CESC was performed (FIG. 20 panel C). These genes were then clustered according to all 26 immune cell subtypes as defined by Porrello et al (73). Notably, the Th1 T cell subtype displayed a significant correlation with SYNGR3 expression in both HPV(+) HNSCC (Log 2FC=3.26, SE=0.17, p-value=9.4E-79, q-value=4.55E-75) and HPV(+) CESC (Log 2FC=1.60, 5E=0.31, p-value=2.75E-07, q-value=6.42E-06) compared to HPV(−) tumors (FIG. 20 panels D and E). These findings suggested that SYNGR3 may be a useful tumor cell extrinsic biomarker for defining HPV status.

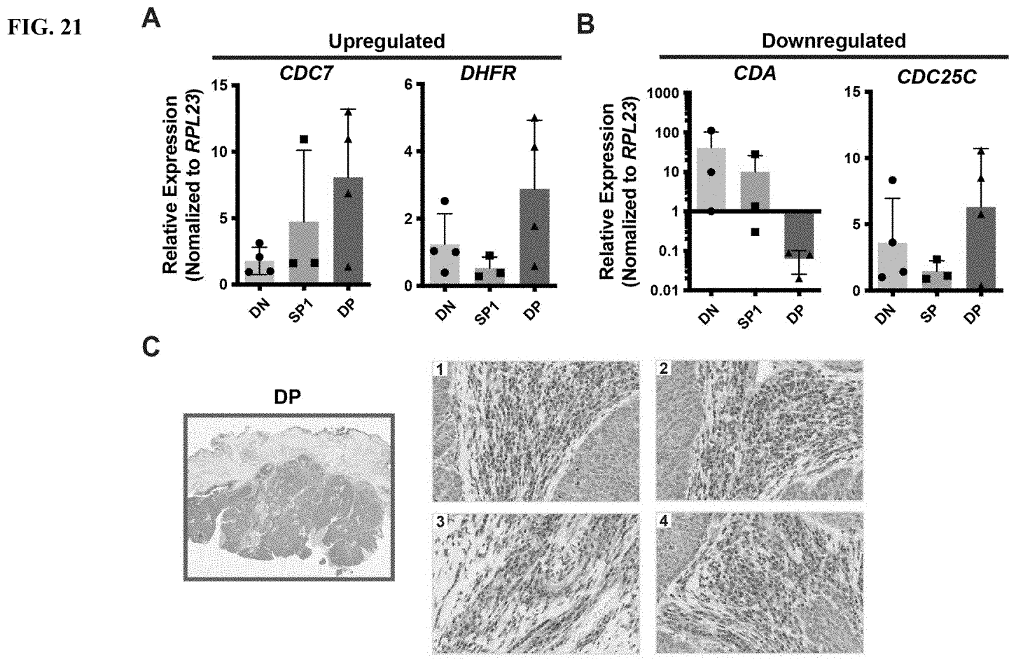

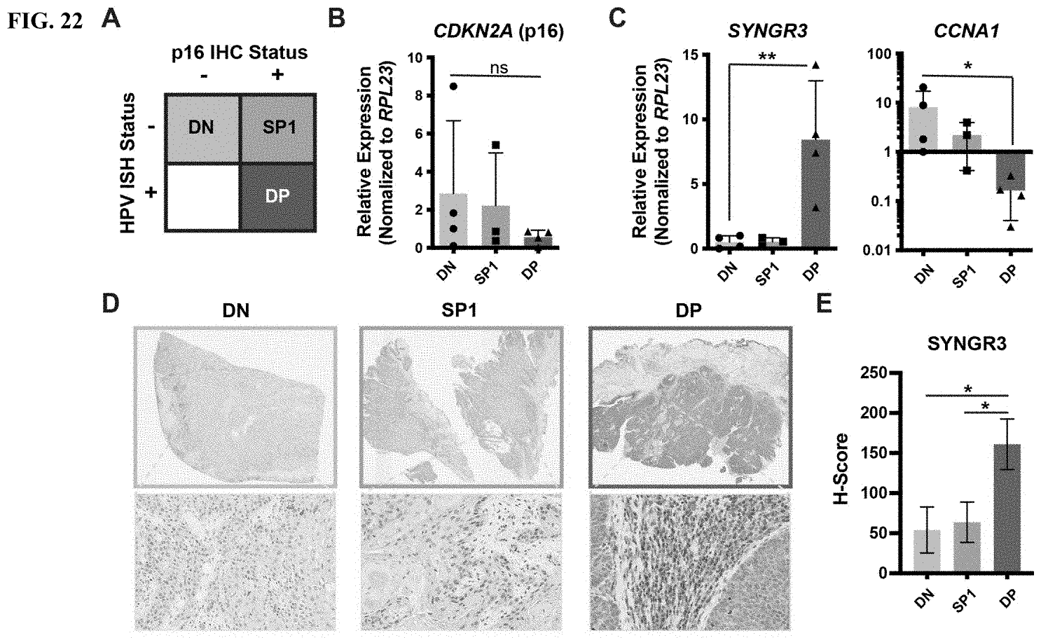

Validation of SYNGR3 expression in HPV(+) HNSCC cohorts: To begin validating the clinical relevance of increased SYNGR3 expression, a cohort of 11 human primary HNSCC samples with available clinical information regarding HPV status were obtained. qPCR was performed on fresh frozen specimens to determine levels of the canonical HPV biomarker p16, as well as other candidate genes identified in FIG. 20 as being differentially expressed specifically in HPV(+) HNSCCs (FIG. 21 panels A-B]). The tumor specimens were separated into three distinct categories based on clinical results from two HPV assays (FIG. 22 panel A): single positive 1 (SP1, positive for p16 IHC; n=3), double positive (DP, positive for both p16 IHC and HPV ISH; n=3), and DN (double negative, n=4). There were no available samples positive for only HPV ISH in this tumor cohort. While mRNA levels of CDKN2A/p16 unexpectedly did not correlate with HPV status (FIG. 22 panel B), a significant upregulation of SYNGR3 mRNA (p<0.01) was observed in DP tumors (FIG. 22 panel C). Alternatively, CCNA1 was identified in bioinformatic analyses as being inversely correlated to HPV status and qPCR confirmed its expression is significantly higher (p<0.05) in the DN group compared to the DP group (FIG. 22 panel C). To validate these findings at the protein level, IHC was next performed on formalin fixed paraffin embedded (FFPE) sections of these tumors. Concordant with the results obtained for mRNA expression, it was found that staining for SYNGR3 protein was significantly higher (p<0.05) in DP samples than in SP1 or DN samples (FIG. 22 panels D and E). Thus, although p16-positive tumors in the cohort were often negative for HPV E6/E7, it was observed that high levels of SYNGR3 were only associated with tumors positive for HPV assays with high specificity (i.e., SP2 and DP tumors).

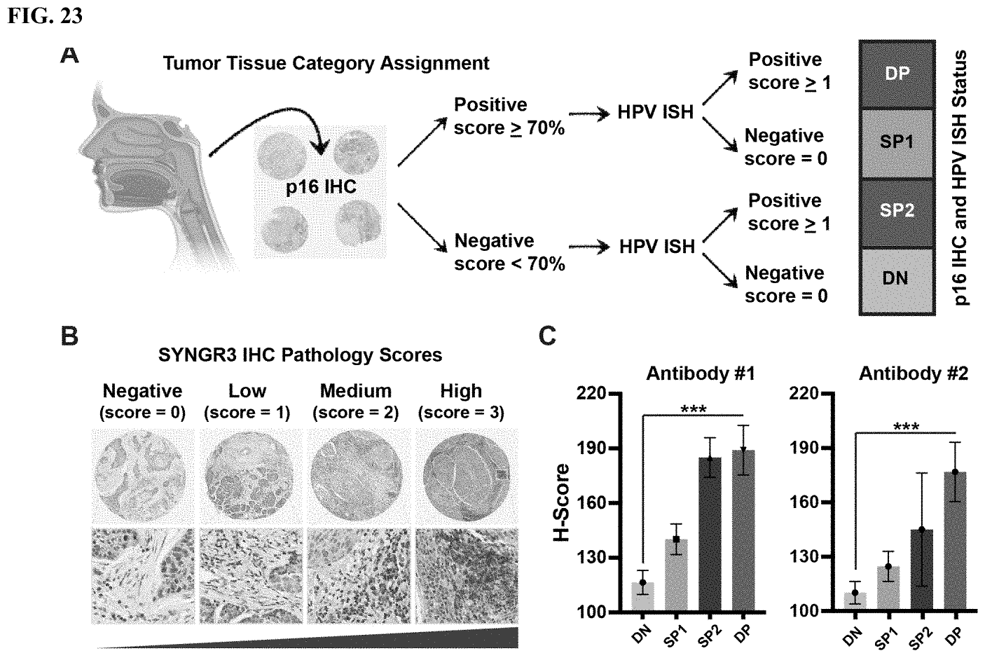

It was sought to extend these findings to a larger panel of HNSCC tumors. SYNGR3 IHC was next performed on a tumor tissue microarray (TMA) that had been previously generated for which the results of HPV clinical assays (p16 IHC and HPV ISH) were available (78, 79, 91). These HNSCC tumor tissues were collected from patients across North Carolina as part of the Carolina Head and Neck Cancer Study (CHANCE) and assigned to categories (e.g., DP, SP1, SP2, or DN) according to the clinical assay results (FIG. 23 panel A; Table 2). Results from both assays were graded by two independent pathologists. The cutoff for p16 positivity was defined as an IHC signal intensity of 1, 2 or 3 in at least 70% of the tumor, and the cutoff for HPV ISH positivity was defined as an ISH signal intensity of 1, 2 or 3 in cell nuclei (FIG. 23 panel B). Using these criteria for HPV marker positivity, we found a similar relationship between increased SYNGR3 expression and HPV marker positivity in this statewide panel of CHANCE tumors using two independent antibodies purchased from different vendors (FIG. 23 panel C) similar to that observed with the smaller cohort (FIG. 22 panels D and E). Notably, despite being the more sensitive assay, p16 IHC is prone to generating false negatives due to sub-optimal specificity for HPV infection. Therefore, the increased sample size offered by the TMA allowed for inclusion of a fourth category of samples with high specificity, SP2 (also referred to as “SP-ISH or ISH-SP”), which are positive for HPV ISH but negative for p16 IHC. Collectively, these data suggest that elevated SYNGR3 has high specificity for HPV(+) HNSCC cases and further support its use as a novel tumor cell extrinsic biomarker of HPV infection.

Co-detection of SYNGR3 IHC and p16 IHC enhances specificity for HPV(+) HNSCC The performance of p16 IHC and SYNGR3 IHC was next evaluated in comparison to a ‘gold standard’ assay for HPV16 DNA that was recently validated in ongoing multi-institutional prospective phase II clinical trials for patients with HPV-associated HNSCC (81, 82, 92, 93). Specifically, the true HPV status was determined on a subset of the CHANCE specimens identified from the TMA by a highly sensitive and specific ddPCR assay for viral genes of high-risk HPV (Table 2). The SYNGR3 IHC assay had very high specificity (89.7%) and positive predictive values (PPV, 82.4%) for HPV16 E7 DNA compared to p16 IHC specificity (72.4%) and PPV (75.0%) in the TMA cohort (Table 3). Moreover, these results highlight the lack of specificity of p16 IHC for HPV detection, as this subset included 11 false positives (Table 3).

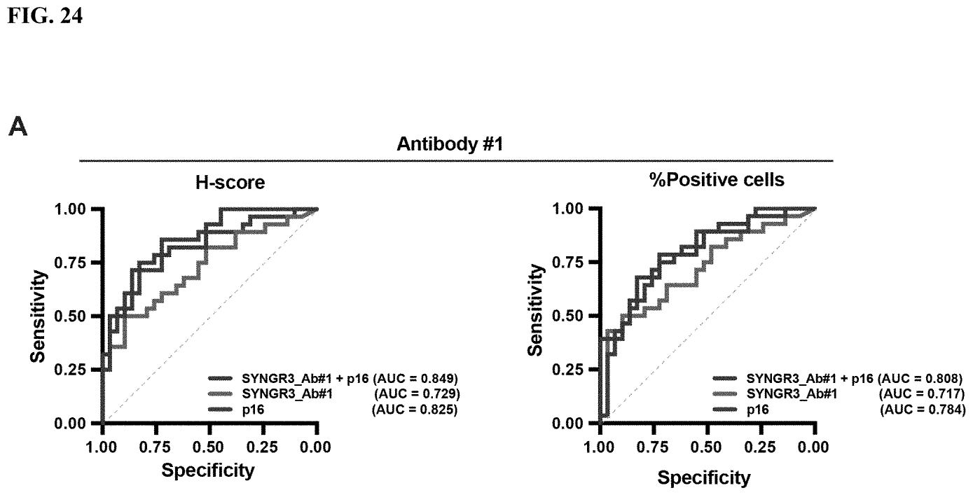

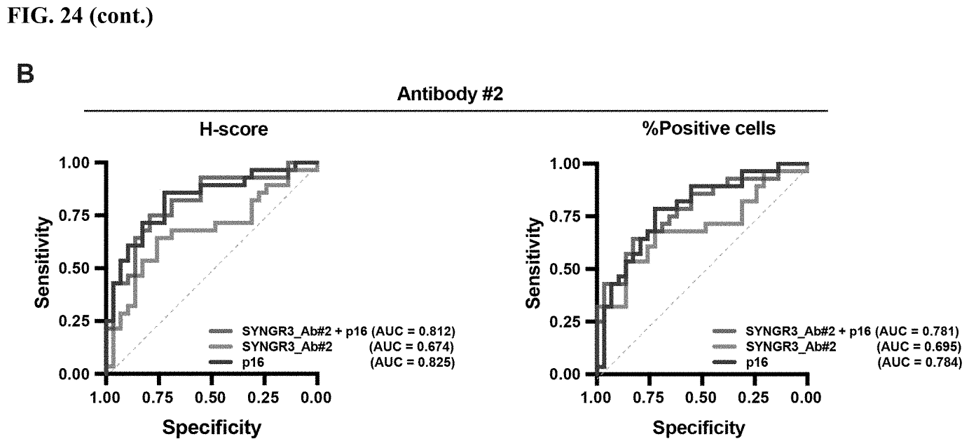

Receiver operating curve (ROC) analyses were next performed to determine the optimal cutoff points for the two different antibodies examined in this study for SYNGR3 IHC interpretation in comparison to p16 IHC (FIG. 24 panels A and B; Table 3). The AUC for H-score (AUC=0.729) was higher for Antibody #1 than that for Percentage Positive stained cells (AUC=0.717), while the AUC for Percentage Positive stained cells (AUC=0.695) for was higher for Antibody #2 than that for H-score (AUC=0.674). Regardless of the single classification method used, the combination of SYNGR3 IHC and p16 IHC was better at discriminating true tumor HPV status with SYNGR3 Antibody #1 (FIG. 24 panel A). An optimal H-score cutoff point of 71.64 on a scale of 0 to 300 for SYNGR3 IHC yielded an average sensitivity of 50.0% and specificity of 89.7% for HPV detection. Thus, when used in combination, the superior specificity of SYNGR3 IHC elevates the diagnostic capabilities of p16 IHC in determining true HPV status, suggesting that comprehensive characterization of the cells expressing SYNGR3 and their location within HPV(+) HNSCCs may benefit its clinical application.

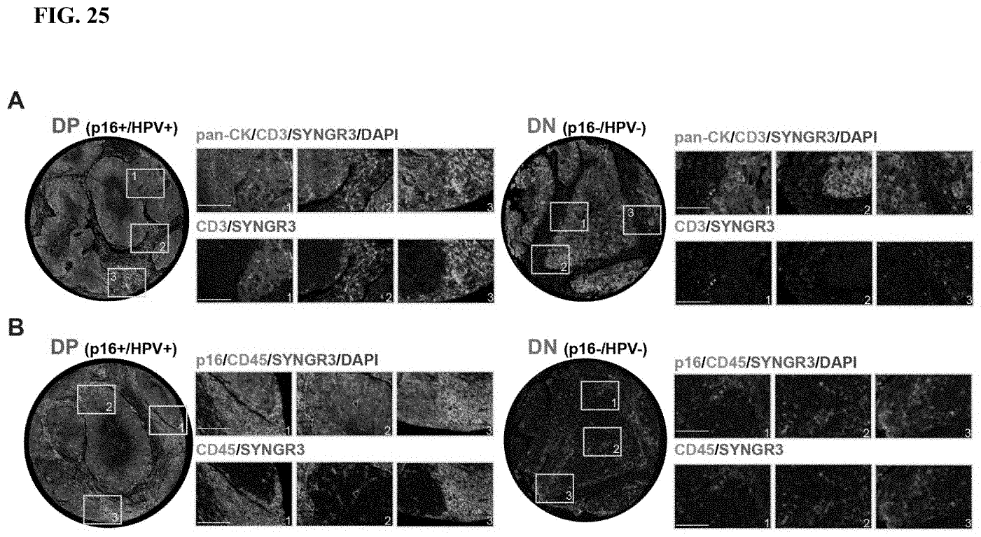

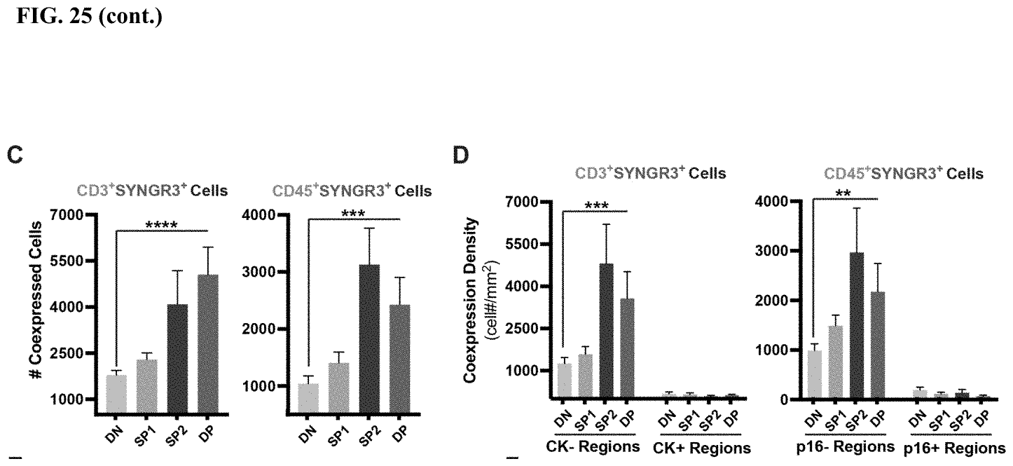

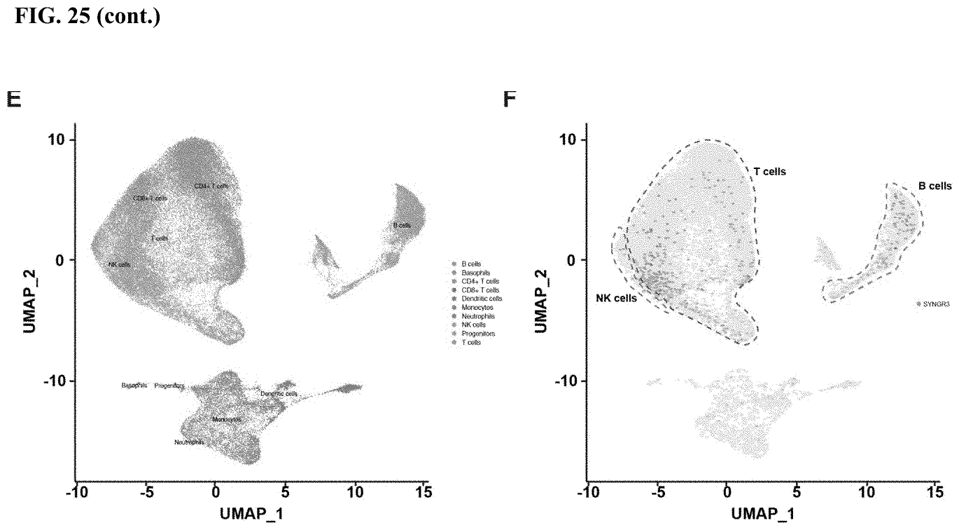

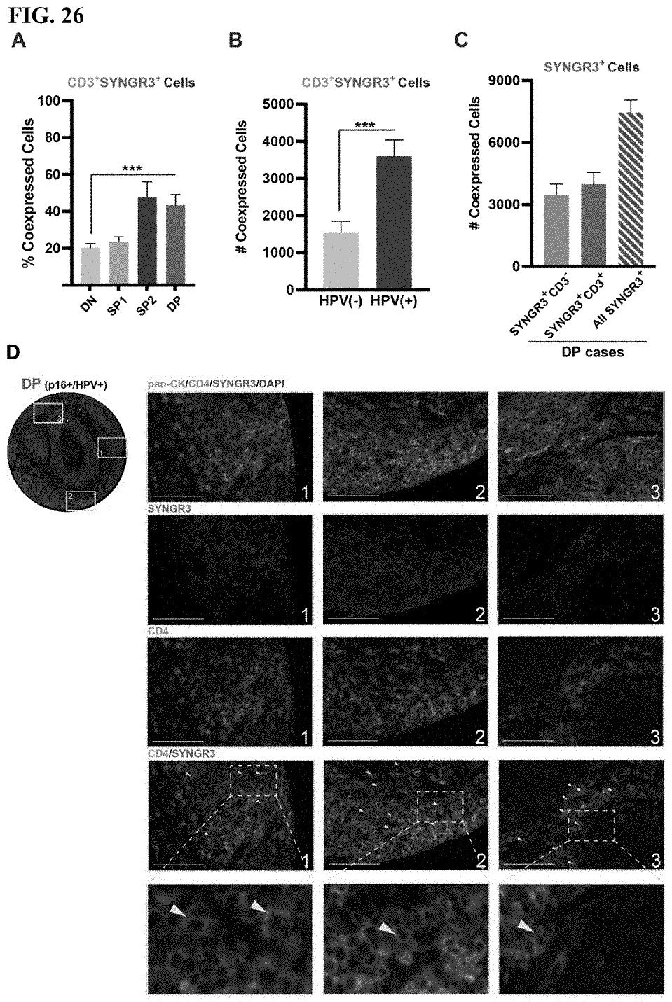

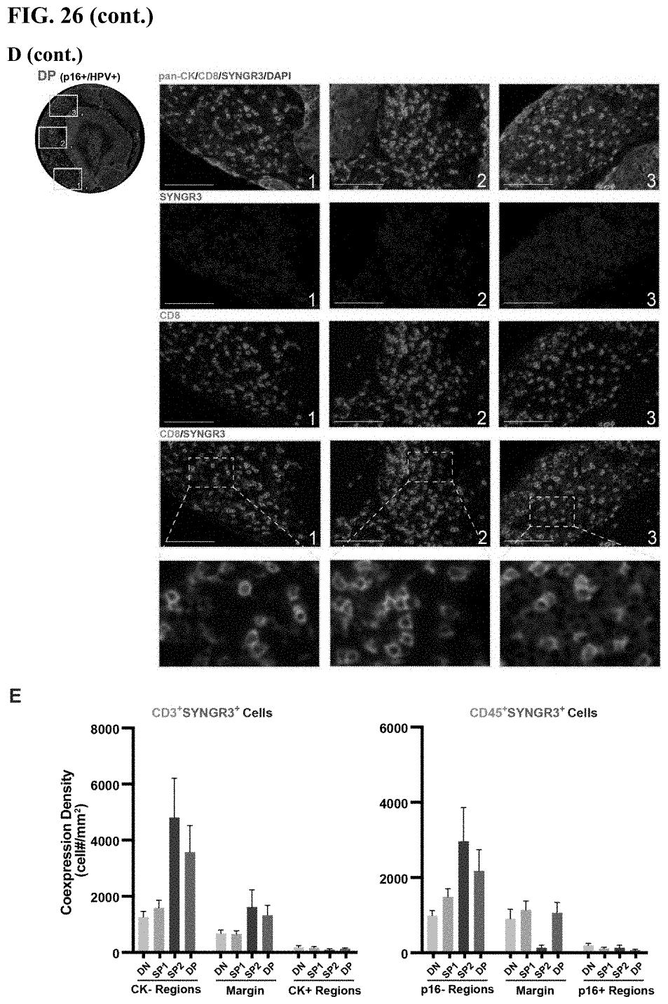

SYNGR3 expression is confined to T and B cells within the tumor stroma: Immunogenomics analyses and initial identification of elevated SYNGR3 expression in HPV(+) HNSCCs indicated that it is highly expressed within the Th1 subset of tumor-infiltrating T cells (FIG. 20 panel C). To examine this more closely and characterize the number and location of SYNGR3+ cells, multiplex IHC was performed to examine SYNGR3 expression in T cells and more generally hematopoietic cells, as well as their distribution within different tumor compartments. First stains were performed for SYNGR3, CD3 (pan-T cell marker), and CK (pan-cytokeratin marker to define the epithelial compartment) and it was found that the number of co-expressed SYNGR3+/CD3+ cells was significantly higher (˜3-fold, p<0.0001) in DP (p16 IHC+/HPV ISH+) tumors compared to DN (p16 IHC−/HPV ISH−) tumors (FIG. 25 panels A and C). The average number (and percentage) of cells co-expressing SYNGR3+/CD3+ in each category are as follows: DN=1787 cells (20.4% of total), SP1=2291 cells (23.4%), SP2=4088 cells (47.6%), and DP=5054 cells (43.3%) (FIG. 25 panel C and FIG. 26 panel A). This difference was validated within the samples with confirmed HPV status by ddPCR (FIG. 26 panel B). Interestingly, only ˜50% of all SYNGR3+ cells were identified as being dual positive SYNGR3+CD3+ cells (FIG. 26 panel C). To validate the immunogenomics analyses, multiplex IHC with additional T cell markers revealed that SYNGR3 expression colocalizes with CD4+ T cells but not with CD8+ cytotoxic T cells within the tumor stroma (FIG. 26 panel D). Collectively, these findings suggest that additional immune cell types may also express SYNGR3.

To examine this further, further stains were performed for SYNGR3, CD45 (pan-hematopoietic cell marker), and p16, and it was found that the number of co-expressed SYNGR3+/CD45+ cells was significantly higher (˜2.5-fold, p<0.001) in DP (p16 IHC+/HPV ISH+) tumors compared to DN (p16 IHC−/HPV ISH−) tumors (FIG. 25 panels B and C). Similarly, the average number of cells co-expressing SYNGR3+/CD45+ in each category was: DN=1042 cells, SP1=1405 cells, SP2=3129 cells, and DP=2425 cells. Region of interest (ROI) analyses of SYNGR3+ cell compartmentalization within the tumor microenvironment were performed by defining the tumor as 25 μM on either side of the border of the tumor core and revealed that SYNGR3+/CD3+ and SYNGR3+/CD45+ cells were primarily confined to stromal regions (pan-CK and p16 negative) compared to the cancer cell islet (pan-CK and p16 positive) regions (FIG. 25 panel D and FIG. 26 panel E). To complement the multiplex IHC analyses and reveal the identity of these SYNGR3+ immune cells, previously published (single-cell RNA sequencing data generated from purified CD45+ cells (i.e., all immune cells) (68) were analyzed for SYNGR3 expression in HPV(+) HNSCCs. Unsupervised clustering of these immune cells confirmed SYNGR3 expression within T cells but also within B cells of HPV(+) HNSCCs (FIG. 25 panels E and F). Notably, B cells and the presence of tertiary lymphoid structures (TLS) can predict immune checkpoint inhibitor efficacy and influence outcome in HNSCC patients, suggesting that detection of SYNGR3 may also have prognostic value.

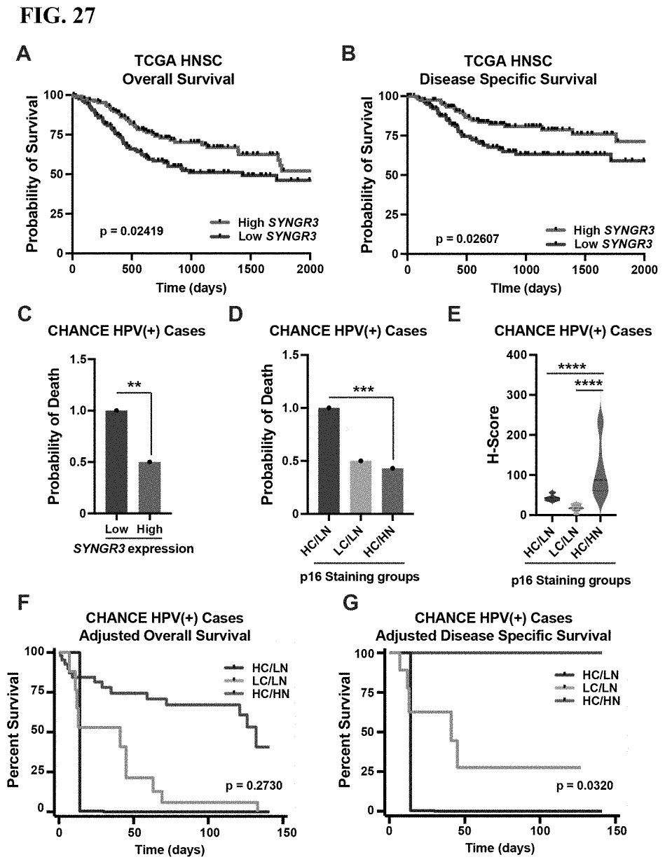

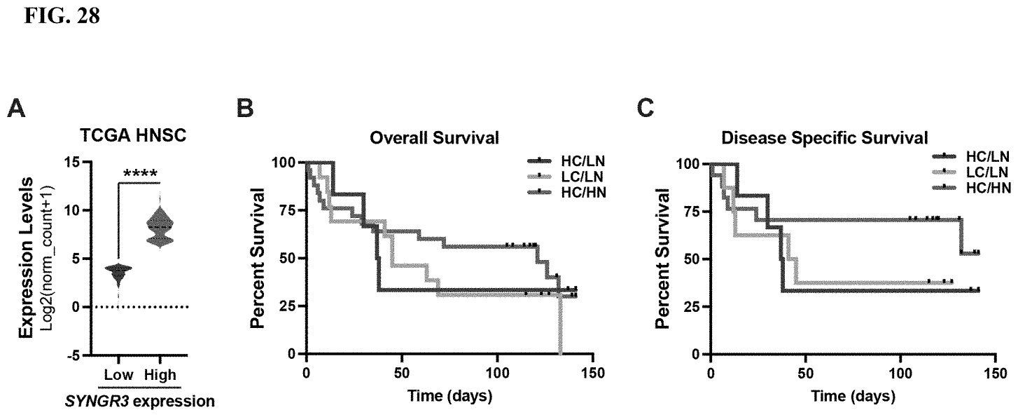

SYNGR3 expression is associated with improved survival: To evaluate the utility of SYNGR3 as a novel prognostic immune cell biomarker of HPV(+) HNSCC, the UCSC Xenabrowser was used to analyze publicly available RNA-seq data for the TCGA head and neck cancer dataset (94). Stratifying samples into SYNGR3 expression quartiles revealed a marked separation in the survival curves such that patients with high SYNGR3 expression show significantly better overall survival (p=0.0242) and disease specific survival (p=0.02607) compared to patients with low SYNGR3 expression (FIG. 27 panels A and B; FIG. 28 panel A). Using the ROC curve analyses to define the optimal cutoff of SYNGR3 positivity (cytoplasmic H-score of 70), it was confirmed that patients included in the TMA with high SYNGR3 expression also had better disease specific survival compared to patients with low SYNGR3 expression (FIG. 27 panel C).

Previous studies demonstrated that p16 cellular localization is an important prognostic biomarker in HNSCC (79, 95). The cytoplasmic and nuclear p16 H-scores of the studies described herein were applied to categorize the HPV(+) HNSCC TMA into the following groups: high cytoplasmic, high nuclear (HC/HN, n=25); low cytoplasmic, low nuclear (LC/LN, n=13); and high cytoplasmic, low nuclear (HC/LN, n=6) (Table 3). This organization of samples reflected those previously published (79), in which the HC/LN group displayed worst disease specific survival (FIG. 27 panel D), with a hazard ratio of 8.6 compared to the HC/HN group (p-value=0.032) (Table 4). SYNGR3 expression was found to be significantly higher in the HN/HC group compared to the HC/LN (p<0.001) and LC/LN (p<0.0001) groups and multivariate analysis demonstrated that this group was associated with significantly improved disease specific survival (FIG. 27 panels E-G and FIG. 28 panels B and C; Table 5). Therefore, SYNGR3 is expressed more highly in the group with improved disease specific survival (HN/HC), suggesting value as a prognostic biomarker in HNSCC patients. Collectively, these findings support a diagnostic and prognostic role for SYNGR3 in HNSCC and potentially other HPV(+) cancers, and indicate that detection of SYNGR3 can be accomplished by pathological analysis of the tumor stroma, making it distinct yet complementary to currently available HPV detection assays.

### Example 3: Methods for Examples 1 and 2

Bioinformatics: Bulk RNA-seq Analysis—The TCGA RNA-seq datasets used in this study were downloaded from The Broad Institute TCGA GDAC Firehose (gdac.broadinsitue.org), which provides TCGA Level 3 data and Level 4 analyses packaged in a form amenable to immediate algorithmic analysis. Specifically, publicly available head and neck squamous cell (HNSCC) tumor data from TCGA was used to evaluate the differential expression of genes between HPV(+) and HPV(−) subjects. HPV(+) samples were defined as having a gene-expression-based ratio E6/E7>0 (n=53), whereas HPV(−) samples were required to have a negative HPV status as determined by both in situ hybridization (ISH) and by p16 immunohistochemistry testing (n=56). Normal samples were omitted from the analysis, according to TCGA records (clin.merged file). Similarly, the cervical squamous cell carcinoma and endocervical adenocarcinoma (CESC) tumor data from TCGA was also used to perform differential expression analysis between HPV(+) (n=281) and HPV(−) (n=22) subjects. HPV status was taken from the available patient HPV test results column in the available merged clinical data. Normal samples were also omitted. Differential expression analysis on both individual bulk RNA-seq data sets was performed using the DESeq2 package in R (71). Differentially expressed genes were defined as having an adjusted p-value <0.05, absolute value of log 2 fold change >1, and baseMean>10. Heatmaps were generated using the ComplexHeatmap package in R (72). The GSE65858 dataset for the OPSCC expression profiles was downloaded from the GEO database (https://www.ncbi.nlm.nih.gov/geo/), and transcript abundance was normalized using quantile normalization. Abundance differences were assessed using Wilcoxon-signed rank test.

Modified ‘Immunome’ Signature—The analyzed immune-related genes were previously identified as belonging to the modified ‘Immunome’ signature’, which can be found in Porrello et al. (73). In particular, genes had to belong to the core portion of this collection of gene sets, which is made up of the following 26 immune cell types: activated dendritic cells (aDC), B cells, CD8 T cells, cytotoxic cells, dendritic cells (DC), eosinophils, immature dendritic cells (iDC), macrophages, mast cells, neutrophils, natural killer (NK) CD56 Bright, NK CD56 Dim, NK Cells, plasmacytoid dendritic cells (pDC), T cells, T helper cells, T (lymphocyte) central memory (Tcm), T (lymphocyte) effector memory (Tem), T (lymphocyte) follicular helper (TFH), T (lymphocyte) gamma delta (Tgd), T helper (type) 1 (Th1), T helper 17 (Th17), T helper (type) 2 (Th2), regulatory T cells (Tregs), immune checkpoints (namely, CD274 (PDL1), CTLA4 and PDCD1 (PD1)), and myeloid-derived suppressor cells (MDSC). Genes belonging to these 26 signatures (577 genes) were included in the summary heat maps only when supported by statistical evidence (false discovery rate (FDR)<0.05) of being differentially expressed in HPV(+) relative to HPV(−) in either bulk RNA-seq dataset.

Single Cell RNA-Seq Analyses—Publicly available HNSCC single cell RNA sequencing data were used to evaluate SYNGR3 expression level in various immune cell types across HPV-positive (n=8) and HPV-negative (n=18) samples (57, 68). Single-cell RNA-sequencing analysis was performed using the Seurat v4 package in R (74). Potential doublets and dying cells were filtered out requiring each cell to have less than 6000 unique features, less than 50,000 mRNAs, and less than 25% mitochondrial gene counts. These filtering criteria resulted in 21,057 cells from the HPV(+) subjects, and 39,919 cells from the HPV(−) subjects. Cell clusters were annotated using the SingleR package (75) using the Monaco immune cell type reference (76).

Clinical Samples: Fresh frozen HNSCC human tumor specimens with affiliated HPV assay clinical diagnoses were obtained. Samples (total n=11) were from the following anatomical sites: larynx (n=2), oral cavity (n=7), and oropharynx (n=2). Histopathologic assessments were made by a pathologist and presented in a non-quantitative, binary format (either negative or positive) for both HPV ISH (high-risk HPV strains) and p16 IHC.

RNA Isolation and Real-Time qPCR: Tissues were homogenized as previously described (77). NucleoZOL (Macherey-Nagel, cat. #: 740404.200) was used in accordance with the manufacturer's instructions to extract RNA from fresh frozen human HNSCC tumors. iScript cDNA Synthesis Kit (BioRad, #1708890) was used to make cDNA from extracted RNA. FastStart Universal SYBR Green Master (Rox) Mix (Roche, cat. #: 04913850001) was used with 1/20 volume of cDNA iScript reaction and 0.25 μM primers. Primer sequences are listed in Table 6. Relative gene expression was determined using the 2ddCt method and normalized using human and mouse RPL23.

Tissue Microarray (TMA): The Carolina Head and Neck Cancer Study (CHANCE) TMA used for these studies includes distinct anatomical locations of the oropharynx, hypopharynx, oral cavity, and larynx and has been thoroughly characterized previously (78). Slides used were reviewed for presence of evaluable tumor. Cores lacking evaluable tumor or with fewer than 500 cells detected by analysis algorithm were excluded. Data presented here include 190 evaluable tumor cores taken from 98 separate tumors (1-3 cores/tumor block). Patient details including sex, race/ethnicity, smoking status, pack years, alcohol use, and diagnosis age are described in Table 2.

HPV In Situ Hybridization: Ventana Benchmark XT autostainer was used for HPV in-situ hybridization (ISH) according to manufacturer's protocol as previously described (79). INFORM HPV III Family 16 Probe (B, Ventana Medical Systems; Tucson Ariz.) was used for staining of HPV strains 16, 18, 31, 33, 35, 39, 45, 51, 52, 56, 58, and 66. Either punctate or diffuse signal pattern in tumor nuclei was scored as positive staining. HPV ISH positive was defined as nuclear score 1-3 at any percent.

Antibodies: Anti-SYNGR3 rabbit polyclonal antibody (referenced as Antibody #1) was purchased from Invitrogen (PAS-60146, Lot182031, Thermo Fisher Scientific; Carlsbad, Calif.). Anti-SYNGR3 (E-11) mouse monoclonal antibody (referenced as Antibody #2) was purchased from Santa Cruz (sc-271046, LotI1718, Santa Cruz Biotechnology, Inc.; Dallas, Tex.). Anti-p16 mouse monoclonal antibody (D-25) used for individual stain was Sigma-Aldrich (MAB4133, Chemicon International Company/Millipore Corporation; Temecula, Calif.); for multiplex staining anti-p16 mouse monoclonal antibody by Ventana (705-4793, Lot Y01733, Ventana Medical; Oro Valley, Ariz.) was used. pan-Cytokeratin rabbit polyclonal antibody used was from Dako (Z0622, Agilent Technologies; Santa Clara, Calif.). A mouse monoclonal antibody to CD3 (NCL-L-CD3-565, Lot6055982) and a CD45 mouse monoclonal antibody (PA0042, Lot66010) from Leica (Leica Microsystems Inc.; Norwell Mass.) were used for multiplex staining.

Immunohistochemistry (IHC): Chromogenic immunohistochemistry (IHC) was performed on paraffin-embedded tissues that were sectioned at 5 micrometers. All IHC was carried out in the Bond III Autostainer (Leica Microsystems Inc.). Slides were dewaxed in Bond Dewax solution (AR9222) and hydrated in Bond Wash solution (AR9590). Antigen retrieval was performed for 20 min at 100° C. in Bond-Epitope Retrieval solution 1, pH-6.0 (AR9961).

Individual stains—For the SYNGR3 Invitrogen antibody slides were incubated for at 1:500 for 1 hr then ImmPress HRP anti-rabbit IgG secondary (MP-7451; Vector Laboratories; Burlingame, Calif.). For the SYNGR3 Santa Cruz antibody slides were incubated at 1:50 for 4 hr followed by Novocastra Post Primary (Leica, #RE7159) and Novolink Polymer (Leica, #RE7161) secondary antibodies for 8 min each. Antibody detection with 3,3′-diaminobenzidine (DAB) was performed using the Bond Intense R detection system (DS9263) with ImmPress HRP anti-rabbit IgG (MP-7451; Vector Laboratories; Burlingame, Calif.).

Multiplex stains—Slides were incubated with SYNGR3 Invitrogen antibody at 1:300 for 1 hr and was detected with ImmPress HRP anti-rabbit IgG and TSA Cy5 (SAT705A001EA; Perkin Elmer, Waltham, Mass.). After completion of SYNGR3 staining, a second round of denaturation (10 min, Bond-Epitope Retrieval solution 1) was followed by incubation in either anti-pan-Cytokeratin (30 min, 1:1500) or CD45 (30 min; ready to use) and detection with ImmPress HRP anti-rabbit IgG and TSA Cy3 (SAT704A001EA; Perkin Elmer). Following pan-Cytokeratin/CD45 staining, a third denaturation step was performed for 10 min in Bond-Epitope Retrieval solution 2 (pH 9.0; AR9640) followed by incubation with either anti-CD3 (1 hr, 1:200) or p16 (1 hr, 1:5) then detection with Bond Polymer (DS9455) and TSA Alexa-488 (B40953, Invitrogen).

Stained slides were dehydrated and coverslipped with either Cytoseal 60 (single DAB stains; 8310-4, Thermo Fisher) or Prolong gold (multiplex stains; P36930, Thermo Fisher). Positive and negative controls (no primary antibody) were included during staining runs. The slides were digitally scanned at 20× magnification using Aperio AT2 (Aperio Technologies, Vista, Calif.) and uploaded to the Aperio eSlideManager database (Leica Biosystems Inc) at the Pathology Services Core at UNC

Interpretation of p16 IHC Histopathology: In addition to the digital image analysis described below, p16 IHC was also previously scored by pathologists for protein expression (79). Each core was scored for cytoplasmic intensity staining and nuclear intensity staining on a 0-3 scale. The percent of positive staining tumor cells was quantified using 10 microscopic fields of 100 cells each. p16 positive was defined as a cytoplasmic or nuclear score of 1-3 in at least 70% cells.

Digital Imaging and Analysis: DAB stained slides for SYNGR3 were digitally scanned using the Aperio ScanScope-XT (serial number ss1475, Aperio Technologies; Vista, Calif.). DAB stained slides for p16 were digitally scanned using Aperio ScanScope CS (serial number ss5072, Leica Biosystems; Buffalo Grove, Ill.). Multiplex immunofluorescent slides were scanned using the Aperio ScanScope FL (serial number ss6132, Leica Biosystems; Buffalo Grove, Ill.). All images were scanned at an apparent 20× magnification and uploaded to the Aperio eSlideManager database (version 12.4.3, Leica Biosystems; Buffalo Grove, Ill.).

Analysis of single stained slides—TMA images stained for p16 or SYNGR3 were digitally segmented into individual cores using TMALab (Aperio Technologies; Vista, Calif.). Whole tissue sections stained were annotated using Aperio ImageScope to remove staining artifacts and tissue folds before they were analyzed. The Cytoplasmic v2 algorithm (Aperio Technologies) was used to analyze p16 and both the Cytoplasmic v2 and the Membrane v9 algorithms (Aperio Technologies) were used to analyze SYNGR3 staining. Using these algorithms, cells were analyzed for DAB signal and the number and percentage of cells with light (1+), medium (2+) and strong (3+) cell staining was determined. H Scores were calculated using the following formula: 3×percentage of strongly staining cells+2×percentage of moderately staining cells+the percentage of weakly staining cells, giving a range of 0 to 300. The average DAB intensities in cells, cytoplasm and nuclei were also determined.

Analysis of multiplex stained slides—TMA images were digitally segmented into individual cores using Tissue Studio in IF TMA mode (Tissue Studio version 2.7 with Tissue Studio Library version 4.4.2; Definiens Inc., Carlsbad Calif.). Cellular Coexpression analysis algorithms were used to quantify the number of cells expressing individual markers, two of 3 markers, and all 3 markers, and cells that were negative for all markers. Tumor Microenvironment analysis algorithm was used to segment cores into regions of interest (ROI) based on designated epithelial marker (pan-CK or p16, respectively) and quantify cell number expressing/coexpressing each marker. The average cytoplasmic intensity was also determined for all ROIs. Tumor margin was defined as 25 μM on either side of the border of the tumor core.

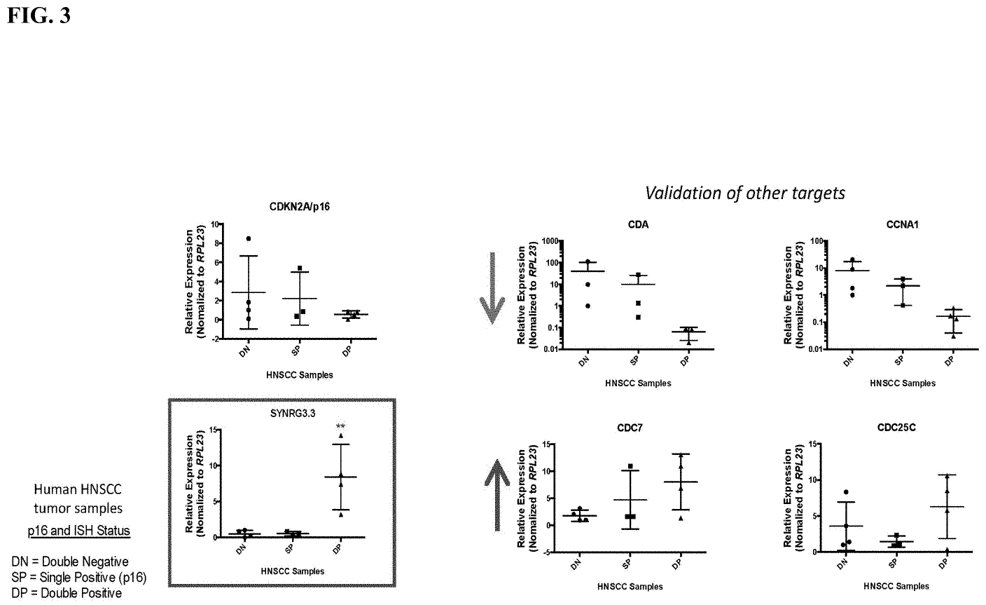

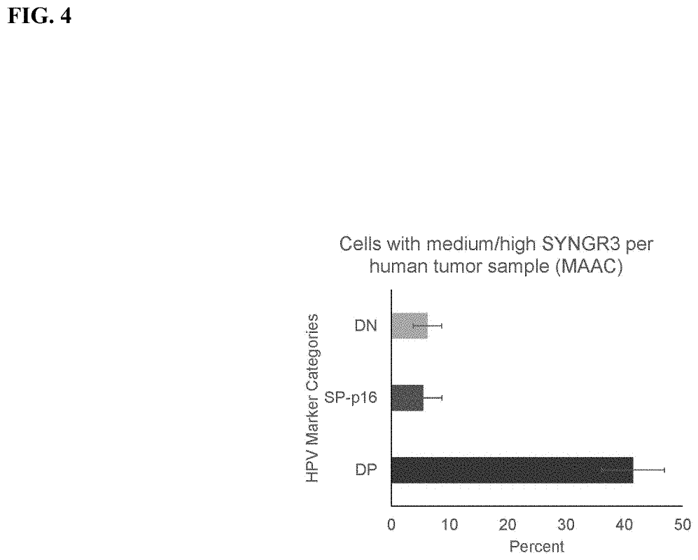

TMA Data Processing: Cores were binned into categories DN, SP-p16 (SP1), SP-ISH (SP2), and DP as described in FIG. 3 and kept these category designations for all future analyses. For multiplex staining, the number of cells coexpressing SYNGR3, CD3, and pan-cytokeratin was added to the number of cells coexpressing only SYNGR3 and CD3 to get the number of total cells coexpressing SYNGR3 and CD3. Similarly, the number of cells coexpressing SYNGR3, CD45, and p16 was added to the number of cells coexpressing only SYNGR3 and CD45 to get the number of total cells coexpressing SYNGR3 and CD45. Total number of cells expressing SYNGR3 was calculated by adding the number of cells expressing SYNGR3 alone, as well as the number of cells in which SYNGR3 was expressed with other marker(s). The number of cells with a nucleus detected in each core was used as the total cell count to calculate percent of cells expressing a particular marker and/or marker coexpression.

For survival analysis, one core per patient tumor block was selected and cores were binned by p16 or SYNGR3 expression from digital image analysis. High p16 cytoplasmic expression was defined as an H-score of 70 or higher; high nuclear expression as a H-score of 100 or higher. High SYNGR3 expression was defined as a cytoplasmic H-score of 70 or higher. For SYNGR3 expression based on p16 staining groups in the HPV(+) TMA, all DN and HPV(−) by ddPCR cores were excluded; any SP1 core that also had a DN core in its same patient block were also excluded.

Receiver Operator Characteristic (ROC) Curve Analyses: ROC curve analysis was carried out to examine and compare the classification accuracy on HNSCC tumor HPV status among several measures, including p16 and SYNGR3 IHC percentage of tumor staining and H score. For each of the classifications measured, the area under the curve (AUC) was calculated using the pROC package in R (80) from which HPV status was determined by ddPCR. The optimal cutoff point was determined by the average sensitivities and specificities.

Survival Analyses: Summary statistics, univariate and bivariate methods including Kaplan-Meier survival analyses and bivariate tests with a significance level alpha set to <0.05, were used to describe the distribution of our patient sample and present their demographic (age, sex, race, smoking status, alcohol status) and clinicopathological (tumor site) characteristics overall and by p16 cytoplasmic/nuclear expression status. Crude 5- and 10-year survival rates by p16 status were also calculated.

Multivariable Cox regression models adjusted for age, sex, race, smoking status, alcohol status, and tumor site were created to estimate the associations of p16 status with overall survival (OS) and disease-specific survival (DSS). To evaluate these associations, we used hazard ratios (HR) and corresponding 95% confidence intervals (CI). All statistical analyses were performed with Stata 16.1 (StataCorp LP, College Station, Tex., USA) and the same program was also used for figure production.

Droplet Digital PCR (ddPCR): HPV was detected by droplet digital PCR on the QX-200 platform (Bio-Rad, Hercules, Calif.) using QuantaSoft software v1.7.4.0917 (Bio-Rad). The assay was performed as previously described (81, 82); details and primer sequences can be found at Chera et al. 2019. The quality of DNA extracted from FFPE tumor blocks was assessed by ddPCR targeting a human genomic locus, estrogen receptor (ESR1) gene. Specific primers and hydrolysis probes were designed to amplify a portion of E6 and E7 genes encoded by HPV16 while only a portion of E7 gene was amplified in case of other high risk HPV strains namely, 18, 31, 33, and 35. Two TMA cores which were not included in ddPCR evaluation but were positive by ISH E6/E7 were included as “true positives” due to the high specificity of HPV ISH. Appropriate HPV plasmid controls were used as positive control for each of the digital PCR assays.

Statistical Analyses: Statistical analyses were performed with GraphPad Prism (version 9) using student's t-test, one-way ANOVA or 2-way ANOVA where applicable. Data are presented as mean±SD or mean±SEM as indicated in the figure legends.

Data Availability Statement: The bioinformatics data analyzed in this study were obtained from the TCGA and the NCBI Gene Expression Omnibus (GEO) at (ncbi.nlm.nih.gov/geo/). The raw IHC data analyzed for this study were generated at the UNC Pathology Services Core.

TABLE 1
SYNGR3 ddPCR results

| Block | ddPCR Status | Category |
|---|---|---|
| 1 | HPV +ve | sp-16 |
| | | dp |
| | | N/A |
| 2 | HPV +ve | dp |
| | | N/A |
| | | sp-16 |
| 3 | HPV +ve | dp |
| | | dp |
| | | sp-ISH |
| 4 | HPV +ve | sp-16 |
| | | dn |
| | | sp-16 |
| 5 | HPV +ve | sp-16 |
| | | sp-16 |
| | | sp-16 |
| 6 | HPV +ve | dp |
| | | sp-ISH |
| | | dp |
| 7 | HPV +ve | dp |
| | | dp |
| | | sp-ISH |
| 8 | HPV +ve | sp-ISH |
| | | dp |
| | | dp |
| 9 | HPV +ve | N/A |
| | | sp-16 |
| | | sp-16 |
| 10 | HPV +ve | N/A |
| | | N/A |
| | | dp |
| 11 | Uninterpretable | N/A |
| | | dn |
| | | dp |
| 12 | HPV +ve | N/A |
| | | N/A |
| | | sp-16 |
| 13 | HPV +ve | N/A |
| | | N/A |
| | | sp-ISH |
| 14 | HPV −ve | |
| | | N/A |
| | | sp-16 |
| 15 | HPV −ve | dn |
| | | sp-16 |
| | | sp-16 |
| 16 | HPV −ve | dn |
| | | dn |
| | | dn |
| 17 | HPV −ve | sp-16 |
| | (Quality of | dn |
| | DNA Low) | sp-16 |
| 18 | HPV −ve | dn |
| | | dn |
| | | dn |
| 19 | HPV −ve | dn |
| | | dn |
| | | sp-16 |
| 20 | HPV −ve | dn |
| | | dn |
| | | dn |
| 21 | HPV −ve | sp-16 |
| | | sp-16 |
| | | dn |
| 22 | HPV −ve | dn |
| | | sp-16 |
| | | sp-16 |
| 23 | HPV −ve | sp-16 |
| | | dn |
| | | sp-16 |
| 24 | HPV −ve | dn |
| | | dn |
| | | dn |

TABLE 2
Demographic and clinical characteristics of the study cases.

| | | | Category | | |
|---|---|---|---|---|---|
| | | | Assignment | | |
| | | DN | SP1 | SP2 | DP |
| | ^{a}N = | ^{b}n = | ^{b}n = | ^{b}n = | ^{b}n = |
| | 98 | 103 | 69 | 5 | 13 |
| Characteristics | N (%) | n (%) | n (%) | n (%) | n (%) |
| Tumor Type | | | | | |
| Hypopharynx | 11 (11.2) | 8 (4.2) | 15 (7.9) | — | — |
| Larynx | 24 (24.5) | 26 (13.7) | 18 (9.5) | — | — |
| Oral | 34 (34.7) | 47 (24.7) | 19 (10) | 1 (0.5) | 3 (1.6) |
| Oropharynx | 27 (27.6) | 22 (11.6) | 17 (8.9) | 4 (2.1) | 10 (5.3) |
| Gender | | | | | |
| Female | 28 (28.6) | 40 (21) | 12 (6.3) | 1 (0.5) | 1 (0.5) |
| Male | 70 (71.4) | 63 (33.2) | 57 (30) | 4 (2.1) | 12 (6.3) |
| Race/Ethnicity | | | | | |
| Asian | 1 (1) | 1 (0.5) | — | — | — |
| Black or African | 31 (31.6) | 33 (17.4) | 25 (13.2) | — | — |
| American | | | | | |
| White | 65 (66.3) | 68 (35.8) | 44 (23.2) | 5 (2.6) | 13 (6.8) |
| Black/African American | 1 (1) | 1 (0.5) | — | — | — |
| and Hispanic/Latino | | | | | |
| Smoking Status | | | | | |
| Never smoker | 8 (8.2) | 7 (3.7) | 4 (2.1) | 1 (0.5) | 4 (2.1) |
| Smoker | 88 (89.8) | 95 (50) | 60 (31.6) | 4 (2.1) | 9 (4.7) |
| Unknown | 2 (2) | 1 (0.5) | 5 (2.6) | — | — |
| ^{c}Pack Years (yr) | | | | | |
| Mean pack years | 39.18 | 36.10 | 40.67 | 34.75 | 39.78 |
| Alcohol Use Status | | | | | |
| Heavy use > 6 months ago | 22 (22.4) | 25 (13.2) | 12 (6.3) | 2 (1.1) | 5 2.6) |
| Heavy use currently: | 18 (18.4) | 15 (7.9) | 16 (8.4) | — | — |
| per >3 drinks day | | | | | |
| Light or moderate | 27 (27.6) | 28 (14.7) | 23 (12.1) | — | 2 (1.1) |
| Never Drinker | 29 (29.6) | 34 (17.9) | 12 (6.3) | 3 (1.6) | 6 (3.2) |
| Unknown | 2 (2) | 1 (0.5) | 5 (2.6) | — | — |
| Diagnosis Age (yr) | | | | | |
| Median | 58 | 58 | 58 | 52 | 54 |
| Mean | 57.46 | 57.95 | 57.93 | 52.00 | 55.62 |
| Range | 20-79 | 24-79 | 20-79 | 45-58 | 44-74 |

^{a}N = 98 tumor blocks;
^{b}n = 190 tumor cores;
^{c}pack years = only smokers included

TABLE 3
Descriptive statistics for HNSCC patients from the
CHANCE study by p16 cytoplasmic/nuclear status.

| | | Category Assignment | | | |
|---|---|---|---|---|---|
| | | HC/HN | HC/LN | LC/LN | |
| | N(%) | n (%) | n (%) | n (%) | P-value |
| | 46 (100) | 25 (54.3) | 6 (13.0) | 13 (28.2) | |
| Sex | | | | | 0.368 |
| Female | 9 (19.6) | 3 (12.0) | 1 (16.6) | 4 (30.8) | |
| Male | 37 (80.4) | 22 (88.0) | 5 (83.3) | 9 (69.2) | |
| Race | | | | | 0.206 |
| Black/African | 14 (30.4) | 7 (28.0) | 0 (0) | 6 (46.2) | |
| American | | | | | |
| White | 32 (69.6) | 18 (72.0) | 6 (13.0) | 7 (53.8) | |
| Alcohol status | | | | | 0.762 |
| heavy use > 6 | 10 (21.7) | 4 (16.0) | 1 (16.6) | 4 (30.8) | |
| months ago | | | | | |
| heavy use | 9 (19.6) | 6 (24.0) | 0 (0) | 3 (23.1) | |
| currently: | | | | | |
| >3 drinks per day | | | | | |
| light or moderate | 13 (28.3) | 9 (36.0) | 2 (33.3) | 2 (15.4) | |
| none | 13 (28.3) | 5 (20.0) | 3 (50) | 4 (30.8) | |
| Unknown | 1 (2.2) | 1 (4.0) | 0 (0) | 0 (0) | |
| Smoking Status | | | | | 0.307 |
| Never smoker | 5 (10.9) | 4 (16.0) | 0 (0) | 0 (0) | |
| Current smoker | 40 (87.0) | 20 (80.0) | 6 (13.0) | 13 (100) | |
| Unknown | 1 (2.2) | 1 (4.0) | 0 (0) | 0 (0) | |
| Tumor site | | | | | 0.075 |
| Hypopharynx | 8 (17.4) | 5 (20.0) | 1 (16.6) | 2 (15.4) | |
| Larynx | 8 (17.4) | 2 (8.0) | 1 (16.6) | 5 (38.5) | |
| Oral | 14 (30.4) | 7 (28.0) | 0 (0) | 6 (46.2) | |
| Oropharynx | 16 (34.8) | 11 (44.0) | 4 (66.7) | 0 (0) | |

TABLE 4
^{a}Association of demographic, clinicopathological variables,
and p16 cytoplasmic/nuclear status with Cox regression
modeling in HNSCC patients from the CHANCE study.

| | OS | | | DSS | | |
|---|---|---|---|---|---|---|
| Characteristic | HR | 95% CI | P | HR | 95% CI | P |
| Sex | | | | | | |
| Female | Ref. | | | Ref. | | |
| Male | 0.36 | 0.10-1.33 | 0.124 | 0.21 | 0.02-1.76 | 0.149 |
| Race | | | | | | |
| Black/African | Ref. | | | Ref. | | |
| American | | | | | | |
| White | 1.13 | 0.39-3.34 | 0.819 | 2.83 | 0.35-23.01 | 0.331 |
| Alcohol status | | | | | | |
| heavy use: | Ref. | | | Ref. | | |
| >6 months ago | | | | | | |
| heavy use | 1.11 | 0.28-4.45 | 0.886 | 11.12 | 1.33-92.85 | 0.026 |
| currently: | | | | | | |
| >3 drinks per day | | | | | | |
| light or moderate | 0.64 | 0.18-2.22 | 0.48 | 1.98 | 0.23-17.40 | 0.537 |
| Never drinker | 0.2 | 0.04-0.92 | 0.039 | 0.19 | 0.02-1.64 | 0.129 |
| Unknown | | | | | | |
| Smoking Status | | | | | | |
| Never smoker | Ref. | | | Ref. | | |
| Current smoker | 0.62 | 0.13-3.10 | 0.562 | 1.96 | 0.12-30.67 | 0.633 |
| Unknown | | | | | | |
| Tumor site | | | | | | |
| Hypopharynx | Ref. | | | Ref. | | |
| Larynx | 0.93 | 0.20-4.28 | 0.925 | 5.41 | 0.32-92.27 | 0.243 |
| Oral | 0.4 | 0.11-1.46 | 0.166 | 4.4 | 0.33-58.41 | 0.261 |
| Oropharynx | 0.42 | 0.09-1.86 | 0.252 | 3.41 | 0.25-46.38 | 0.357 |
| SYNGR3 status | | | | | | |
| HC/HN | Ref. | | | Ref. | | |
| HC/LN | 2.15 | 0.55-8.48 | 0.273 | 8.6 | 1.21-61.25 | 0.032 |
| LC/LN | 1.25 | 0.42-3.72 | 0.687 | 2.87 | 0.45-18.11 | 0.263 |

^{a}p16 cytoplasmic/nuclear status HRs are adjusted for sex, race, smoking, alcohol intake and tumor site

TABLE 5
Crude 5- and 10-year survival rates
by p16 cytoplasmic/nuclear status.

| | | Category Assignment | | |
|---|---|---|---|---|
| | | HC/HN | HC/LN | LC/LN |
| 5-Year Crude | OS | 60 | 33 | 46 |
| Survival (%) | DS | 71 | 33 | 38 |
| 10-Year Crude | OS | 56 | 33 | 31 |
| Survival (%) | DS | 71 | 33 | 38 |

TABLE 6
List of Primers.

| Target | Sequence (5′-3′) | SEQ ID NO | |
|---|---|---|---|
| Hs_CDK2N2A (p16) | GTG GAC CTG GCT GAG | 1 | (forward) |
| | CTT TCA ATC GGG GAT GTC TG | 2 | (reverse) |
| Hs_SYNGR3 | ATGTGCGCTTCCAGCAAATC | 3 | (forward) |
| | ACCACAGGAAGGACCAGAGT | 4 | (reverse) |
| Hs_CCNA1 | TCA GTA CCT TAG GGA AGC TGA AA | 5 | (forward) |
| | CCA GTC CAC CAG AAT CGT G | 6 | (reverse) |
| Hs_CDC7 | TTCAGTGCCTAACAGTGGCT | 7 | (forward) |
| | GAATGTCCAAAAACGACTCATGCT | 8 | (reverse) |
| Hs_DHFR | CAACCTCTTCAGTAGAAGGTAAACA | 9 | (forward) |
| | TGCCACCAACTATCCAGACC | 10 | (reverse) |
| Hs_CDA | TGCCCCTACAGTCACTTTCC | 11 | (forward) |
| | AGCAGGCATTTTCTATGTTGC | 12 | (reverse) |
| Hs_CDC25C | AGGGGCACCTGATTGGTGAT | 13 | (forward) |
| | CAGCCACTGTTTCTGGGTTG | 14 | (reverse) |
| Mm_Rpl23 | GGC AAA CCA GAA CTA AGG AAA A | 15 | (forward) |
| | TTC GAT ATG ACT TTC GTT GTC G | 16 | (reverse) |

### REFERENCES FOR EXAMPLES 1-3

1. F. Bray, J. Ferlay, I. Soerjomataram, R. L. Siegel, L. A. Torre, A. Jemal, Global cancer statistics 2018: GLOBOCAN estimates of incidence and mortality worldwide for 36 cancers in 185 countries. Ca Cancer J Clin. 68, 394-424 (2018).
2. G. B. of D. C. Collaboration, C. Fitzmaurice, C. Allen, R. M. Barber, L. Barregard, Z. A. Bhutta, H. Brenner, D. J. Dicker, O. Chimed-Orchir, R. Dandona, L. Dandona, T. Fleming, M. H. Forouzanfar, J. Hancock, R. J. Hay, R. Hunter-Merrill, C. Huynh, H. D. Hosgood, C. O. Johnson, J. B. Jonas, J. Khubchandani, G. A. Kumar, M. Kutz, Q. Lan, H. J. Larson, X. Liang, S. S. Lim, A. D. Lopez, M. F. Maclntyre, L. Marczak, N. Marquez, A. H. Mokdad, C. Pinho, F. Pourmalek, J. A. Salomon, J. R. Sanabria, L. Sandar, B. Sartorius, S. M. Schwartz, K. A. Shackelford, K. Shibuya, J. Stanaway, C. Steiner, J. Sun, K. Takahashi, S. E. Vollset, T. Vos, J. A. Wagner, H. Wang, R. Westerman, H. Zeeb, L. Zoeckler, F. Abd-Allah, M. B. Ahmed, S. Alabed, N. K. Alam, S. F. Aldhahri, G. Alem, M. A. Alemayohu, R. Ali, R. Al-Raddadi, A. Amare, Y. Amoako, A. Artaman, H. Asayesh, N. Atnafu, A. Awasthi, H. B. Saleem, A. Barac, N. Bedi, I. Bensenor, A. Berhane, E. Bernabé, B. Betsu, A. Binagwaho, D. Boneya, I. Campos-Nonato, C. Castañeda-Orjuela, F. Catalá-López, P. Chiang, C. Chibueze, A. Chitheer, J.-Y. Choi, B. Cowie, S. Damtew, J. das Neves, S. Dey, S. Dharmaratne, P. Dhillon, E. Ding, T. Driscoll, D. Ekwueme, A. Y. Endries, M. Farvid, F. Farzadfar, J. Fernandes, F. Fischer, T. T. G/hiwot, A. Gebru, S. Gopalani, A. Hailu, M. Horino, N. Horita, A. Husseini, I. Huybrechts, M. Inoue, F. Islami, M. Jakovljevic, S. James, M. Javanbakht, S. H. Jee, A. Kasaeian, M. S. Kedir, Y. S. Khader, Y.-H. Khang, D. Kim, J. Leigh, S. Linn, R. Lunevicius, H. M. A. E. Razek, R. Malekzadeh, D. C. Malta, W. Marcenes, D. Markos, Y. A. Melaku, K. G. Meles, W. Mendoza, D. T. Mengiste, T. J. Meretoja, T. R. Miller, K. A. Mohammad, A. Mohammadi, S. Mohammed, M. Moradi-Lakeh, G. Nagel, D. Nand, Q. L. Nguyen, S. Nolte, F. A. Ogbo, K. E. Oladimeji, E. Oren, M. Pa, E.-K. Park, D. M. Pereira, D. Plass, M. Qorbani, A. Radfar, A. Rafay, M. Rahman, S. M. Rana, K. Søreide, M. Satpathy, M. Sawhney, S. G. Sepanlou, M. A. Shaikh, J. She, I. Shiue, H. R. Shore, M. G. Shrime, S. So, S. Soneji, V. Stathopoulou, K. Stroumpoulis, M. B. Sufiyan, B. L. Sykes, R. Tabarés-Seisdedos, F. Tadese, B. A. Tedla, G. A. Tessema, J. S. Thakur, B. X. Tran, K. N. Ukwaja, B. S. C. Uzochukwu, V. V. Vlassov, E. Weiderpass, M. W. Terefe, H. G. Yebyo, H. H. Yimam, N. Yonemoto, M. Z. Younis, C. Yu, Z. Zaidi, M. E. S. Zaki, Z. M. Zenebe, C. J. L. Murray, M. Naghavi, Global, Regional, and National Cancer Incidence, Mortality, Years of Life Lost, Years Lived With Disability, and Disability-Adjusted Life-years for 32 Cancer Groups, 1990 to 2015: A Systematic Analysis for the Global Burden of Disease Study. Jama Oncol. 3, 524 (2016).
3. D. E. Johnson, B. Burtness, C. R. Leemans, V. W. Y. Lui, J. E. Bauman, J. R. Grandis, Head and neck squamous cell carcinoma. Nat Rev Dis Primers. 6, 92 (2020).
4. R. L. Siegel, K. D. Miller, H. E. Fuchs, A. Jemal, Cancer Statistics, 2021. Ca Cancer J Clin. 71, 7-33 (2021).
5. A. G. Sacco, E. E. Cohen, Current treatment options for recurrent or metastatic head and neck squamous cell carcinoma. J Clin Oncol. 33, 3305-3315 (2015).
6. N. C. C. Network, NCCN Clinical Practice Guidelines in Oncology (NCCN Guidelines®)—Head and Neck Cancers. NCCN.org. 3.2021 (2021).
7. R. J. C. Slebos, Y. Yi, K. Ely, J. Carter, A. Evj en, X. Zhang, Y. Shyr, B. M. Murphy, A. J. Cmelak, B. B. Burkey, J. L. Netterville, S. Levy, W. G. Yarbrough, C. H. Chung, Gene expression differences associated with human papillomavirus status in head and neck squamous cell carcinoma. Clin Cancer Res. 12, 701-709 (2006).
8. C. H. Chung, J. S. Parker, G. Karaca, J. Wu, W. K. Funkhouser, D. Moore, D. Butterfoss, D. Xiang, A. Zanation, X. Yin, W. W. Shockley, M. C. Weissler, L. G. Dressler, C. G. Shores, W. G. Yarbrough, C. M. Perou, Molecular classification of head and neck squamous cell carcinomas using patterns of gene expression. Cancer Cell. 5, 489-500 (2004).
9. T. C. G. A. Network, Comprehensive genomic characterization of head and neck squamous cell carcinomas. Nature. 517, 576-582 (2015).
10. P. M. Weinberger, Z. Yu, B. G. Haffty, D. Kowalski, M. Harigopal, J. Brandsma, C. Sasaki, J. Joe, R. L. Camp, D. L. Rimm, A. Psyrri, Molecular classification identifies a subset of human papillomavirus-associated oropharyngeal cancers with favorable prognosis. J Clin Oncol. 24, 736-747 (2006).
11. F. D. Nicole, R. Pavithra, S. Varun, P. T. Alexander, M. T. Kathleen, J. D. Claire, G.-N. O. Frederico, W. S. Gregory, J. Jalal, L. Alexander, R. K. Anil, N. Hiroshi, C. A. Joseph, P. R. Curtis, W. A. Elizabeth, W. E. Bradford, M. M. Iain, C. B. Roger, G. A. Phyllis, B. Devraj, Identifying predictors of HPV-related head and neck squamous cell carcinoma progression and survival through patient-derived models. Int J Cancer. 147, 3236 (2020).
12. C. Fakhry, W. H. Westra, S. Li, A. Cmelak, J. A. Ridge, H. Pinto, A. Forastiere, M. L. Gillison, Improved Survival of Patients With Human Papillomavirus—Positive Head and Neck Squamous Cell Carcinoma in a Prospective Clinical Trial. Jnci J National Cancer Inst. 100, 261-269 (2008).
13. K. K. Ang, J. Harris, R. Wheeler, R. Weber, D. I. Rosenthal, P. F. Nguyen-Tan, W. H. Westra, C. H. Chung, R. C. Jordan, C. Lu, H. Kim, R. Axelrod, C. C. Silverman, K. P. Redmond, M. L. Gillison, Human Papillomavirus and Survival of Patients with Oropharyngeal Cancer. New Engl J Medicine. 363, 24-35 (2010).
14. I. A. F. R. O. CANCER, IARC Monographs on the Evaluation of Carcinogenic Risks to Humans VOLUME 90 Human Papillomaviruses (International Agency for Research on Cancer, Lyon, France, ed. 90).
15. C. C. R. Ragin, E. Taioli, Survival of squamous cell carcinoma of the head and neck in relation to human papillomavirus infection: Review and meta-analysis. Int J Cancer. 121, 1813-1820 (2007).
16. S. Marur, G. D. Souza, W. H. Westra, A. A. Forastiere, HPV-associated head and neck cancer: a virus-related cancer epidemic. Lancet Oncol. 11, 781-789 (2010).
17. D. C. Marshall, D. D. Kao, R. Bakst, S. Sharma, R. Ferrandino, K. Rosenzweig, J. Wisnivesky, K. Sigel, Prognostic role of human papilloma virus status in hypopharyngeal squamous cell carcinoma. Laryngoscope Investigative Otolaryngology. 5, 860-867 (2020).
18. S. Holzhauser, E. Pirotte, J. Jones, D. Owens, A. Al-Hussaini, P. Giles, M. Evans, S. Man, N. Powell, Sensitivity of human papillomavirus-positive and -negative oropharyngeal cancer cell lines to ionizing irradiation. Oncol Rep. 44, 1717-1726 (2020).
19. M. C. de Jong, J. Pramana, J. L. Knegjens, A. J. M. Balm, M. W. M. van den Brekel, M. Hauptmann, A. C. Begg, C. R. N. Rasch, HPV and high-risk gene expression profiles predict response to chemoradiotherapy in head and neck cancer, independent of clinical factors. Radiother Oncol. 95, 365-370 (2010).
20. S. Connor, M. Anjari, C. Burd, A. Guha, M. Lei, T. Guerrero-Urbano, I. Pai, P. Bassett, V. Goh, The impact of human papilloma virus status on the prediction of head and neck cancer chemoradiotherapy outcomes using the pre-treatment apparent diffusion coefficient. Br J Radiology. 94, 20210333 (2021).
21. H. Mirghani, F. Amen, P. Blanchard, F. Moreau, J. Guigay, D. M. Hartl, J. L. S. Guily, Treatment de-escalation in HPV-positive oropharyngeal carcinoma: Ongoing trials, critical issues and perspectives. Int J Cancer. 136, 1494-1503 (2015).
22. P. Gabani, A. J. Lin, J. Barnes, P. Oppelt, D. R. Adkins, J. T. Rich, J. P. Zevallos, M. D. Daly, H. A. Gay, W. L. Thorstad, Radiation therapy dose de-escalation compared to standard dose radiation therapy in definitive treatment of HPV-positive oropharyngeal squamous cell carcinoma. Radiother Oncol. 134, 81-88 (2019).
23. J. Durkova, M. Boldis, S. Kovacova, Has the time come for de-escalation in the management of oropharyngeal carcinoma? Biomed Pap. 163, 293-301 (2019).
24. G. T. Stock, R. R. C. C. Bonadio, G. D. Castro, De-escalation treatment of human papillomavirus-positive oropharyngeal squamous cell carcinoma: An evidence-based review for the locally advanced disease. Curr Opin Oncol. 30, 146-151 (2018).
25. N. Shaverdian, J. V. Hegde, C. Felix, S. Hsu, V. Basehart, M. L. Steinberg, A. M. Chen, Patient perspectives and treatment regret after de-escalated chemoradiation for human papillomavirus-positive oropharyngeal cancer: Findings from a phase II trial. Head Neck. 41, 2768-2776 (2019).
26. S. Wagner, E.-S. Prigge, N. Wuerdemann, H. Reder, A. Bushnak, S. J. Sharma, T. Obermueller, M. von K. Doeberitz, T. Dreyer, S. Gattenlohner, G. Wolf, J. Pons-Ktihnemann, C. Wittekindt, J. P. Klussmann, Evaluation of p16INK4a expression as a single marker to select patients with HPV-driven oropharyngeal cancers for treatment de-escalation. Brit J Cancer. 123, 1114-1122 (2020).
27. J. V. Hegde, N. Shaverdian, M. E. Daly, C. Felix, D. L. Wong, M. H. Rosove, J. H. Garst, P. Wang, D. Veruttipong, S. Rao, R. C. Fragoso, J. W. Riess, M. L. Steinberg, A. M. Chen, Patient-reported quality-of-life outcomes after de-escalated chemoradiation for human papillomavirus-positive oropharyngeal carcinoma: Findings from a phase 2 trial. Cancer. 124, 521-529 (2018).
28. A. Rühle, A.-L. Grosu, N. H. Nicolay, De-Escalation Strategies of (Chemo)Radiation for Head-and-Neck Squamous Cell Cancers—HPV and Beyond. Cancers. 13, 2204 (2021).
29. S. J. Smeets, A. T. Hesselink, E. J. M. Speel, A. Haesevoets, P. J. F. Snijders, M. Pawlita, C. J. L. M. Meijer, B. J. M. Braakhuis, C. R. Leemans, R. H. Brakenhoff, A novel algorithm for reliable detection of human papillomavirus in paraffin embedded head and neck cancer specimen. Int J Cancer. 121, 2465-2472 (2007).
30. R. L. Cantley, E. Gabrielli, F. Montebelli, D. Cimbaluk, P. Gattuso, G. Petruzzelli, Ancillary Studies in Determining Human Papillomavirus Status of Squamous Cell Carcinoma of the Oropharynx: A Review. Pathology Res Int. 2011, 1-7 (2011).
31. C. T. Allen, J. S. Lewis, S. K. El-Mofty, B. H. Haughey, B. Nussenbaum, Human papillomavirus and oropharynx cancer: Biology, detection and clinical implications. Laryngoscope. 120, 1756-1772 (2010).
32. P.-Y. Perera, L. P. Perera, L. Filkoski, W. Chen, J. H. Lichy, E. Paal, J. H. Maxwell, Inclusion of an E7 DNA Amplification Test Improves the Robustness of Human Papillomavirus-Associated Oropharyngeal Squamous Cell Carcinoma Diagnosis. World J Oncol. 11, 1-8 (2020).
33. A. G. Schache, T. Liloglou, J. M. Risk, A. Filia, T. M. Jones, J. Sheard, J. A. Woolgar, T. R. Helliwell, A. Triantafyllou, M. Robinson, P. Sloan, C. Harvey-Woodworth, D. Sisson, R. J. Shaw, Evaluation of Human Papilloma Virus Diagnostic Testing in Oropharyngeal Squamous Cell Carcinoma: Sensitivity, Specificity, and Prognostic Discrimination. Clin Cancer Res. 17, 6262-6271 (2011).
34. A. Lenhoff, Five FDA-approved HPV assays. Mlo Medical Laboratory Observer. 44, 14, 16, 18 (2012).
35. S. Yang, R. E. Rothman, PCR-based diagnostics for infectious diseases: uses, limitations, and future applications in acute-care settings. Lancet Infect Dis. 4, 337-48 (2004).
36. F. Z. Marino, A. Ronchi, M. Stilo, I. Cozzolino, E. L. Mantia, N. Colacurci, G. Colella, R. Franco, Multiplex HPV RNA in situ hybridization/p16 immunohistochemistry: a novel approach to detect papillomavirus in HPV-related cancers. A novel multiplex ISH/IHC assay to detect HPV. Infect Agents Cancer. 15, 46 (2020).
37. A. L. P. Abreu, R. P. Souza, F. Gimenes, M. E. L. Consolaro, A review of methods for detect human Papillomavirus infection. Virol J. 9, 262-262 (2012).
38. R. C. Jordan, M. W. Lingen, B. Perez-Ordonez, X. He, R. Pickard, M. Koluder, B. Jiang, P. Wakely, W. Xiao, M. L. Gillison, Validation of Methods for Oropharyngeal Cancer HPV Status Determination in US Cooperative Group Trials. Am J Surg Pathology. 36, 945-954 (2012).
39. M. Lechner, A. R. Chakravarthy, V. Walter, L. Masterson, A. Feber, A. Jay, P. M. Weinberger, R. A. Mclndoe, C. T. Forde, K. Chester, N. Kalavrezos, P. O'Flynn, M. Forster, T. M. Jones, F. M. Vaz, D. N. Hayes, T. R. Fenton, Frequent HPV-independent p16/INK4A overexpression in head and neck cancer. Oral Oncol. 83, 32-37 (2018).
40. E. S. Prigge, M. Arbyn, M. von K. Doeberitz, M. Reuschenbach, Diagnostic accuracy of p16INK4a immunohistochemistry in oropharyngeal squamous cell carcinomas: A systematic review and meta-analysis. Int J Cancer. 140, 1186-1198 (2017).
41. Z. W. Chen, I. Weinreb, S. Kamel-Reid, B. Perez-Ordoñez, Equivocal p16 Immunostaining in Squamous Cell Carcinoma of the Head and Neck: Staining Patterns are Suggestive of HPV Status. Head Neck Pathology. 6, 422-429 (2012).
42. C. Paschalis, K. Michail, K. Maria, G. Alexandra, P16 detection in benign, precursor epithelial lesions and carcinomas of head and neck. Pathology—Res Pract. 216, 153035 (2020).
43. B. L. Wilson, A.-K. Israel, M. G. Ettel, A. A. L. Limbach, ROC analysis of p16 expression in cell blocks of metastatic head and neck squamous cell carcinoma. J Am Soc Cytopathol. 10, 423-428 (2021).
44. H. Wang, Y. Zhang, W. Bai, B. Wang, J. Wei, R. Ji, Y. Xin, L. Dong, X. Jiang, Feasibility of Immunohistochemical p16 Staining in the Diagnosis of Human Papillomavirus Infection in Patients With Squamous Cell Carcinoma of the Head and Neck: A Systematic Review and Meta-Analysis. Frontiers Oncol. 10, 1-13 (2020).
45. A. Shan, L. M. Rooper, J. F. Ryan, D. W. Eisele, C. Fakhry, p16 immunohistochemistry for primary tumor detection in HPV-positive squamous cell carcinoma of unknown primary. Am J Otolaryng. 42, 103015 (2021).
46. A. D. Singhi, W. H. Westra, Comparison of human papillomavirus in situ hybridization and p16 immunohistochemistry in the detection of human papillomavirus-associated head and neck cancer based on a prospective clinical experience. Cancer. 116, 2166-2173 (2010).
47. J. S. Lewis, B. Beadle, J. A. Bishop, R. D. Chernock, C. Colasacco, C. Lacchetti, J. T. Moncur, J. W. Rocco, M. R. Schwartz, R. R. Seethala, N. E. Thomas, W. H. Westra, W. C. Faquin, Human papillomavirus testing in head and neck carcinomas guideline from the college of American pathologists. Arch Pathol Lab Med. 142, 559-597 (2018).
48. E. S. Keung, R. J. Souers, J. A. Bridge, W. C. Faquin, R. P. Graham, M. R. Hameed, J. S. Lewis, J. D. Merker, P. Vasalos, J. T. Moncur, Comparative performance of high-risk human papillomavirus RNA and DNA in situ hybridization on college of American pathologists proficiency tests. Arch Pathol Lab Med. 144, 344-349 (2020).
49. K. Suresh, P. V. Shah, S. Coates, B. A. Alexiev, S. Samant, In situ hybridization for high risk HPV E6/E7 mRNA in oropharyngeal squamous cell carcinoma. Am J Otolaryng. 42, 102782 (2021).
50. J. Chi, I. R. Preeshagul, S. Sheikh-Fayyaz, S. Teckie, N. Kohn, Y. Ziemba, A. Laser, D. Frank, M. Ghaly, D. Kamdar, D. Kraus, D. Paul, N. Seetharamu, Evaluating of HPV-DNA ISH as an adjunct to p16 testing in oropharyngeal cancer. Futur Sci Oa. 6, FS0606 (2020).
51. S. Begum, M. L. Gillison, T. L. Nicol, W. H. Westra, Detection of human papillomavirus-16 in fine-needle aspirates to determine tumor origin in patients with metastatic squamous cell carcinoma of the head and neck. Clin Cancer Res. 13, 1186-1191 (2007).
52. J. A. Bishop, X.-J. Ma, H. Wang, Y. Luo, P. B. Illei, S. Begum, J. M. Taube, W. M. Koch, W. H. Westra, Detection of Transcriptionally Active High-risk HPV in Patients With Head and Neck Squamous Cell Carcinoma as Visualized by a Novel E6/E7 mRNA In Situ Hybridization Method. Am J Surg Pathology. 36, 1874-1882 (2012).
53. S. Thavaraj, A. Stokes, E. Guerra, J. Bible, E. Halligan, A. Long, A. Okpokam, P. Sloan, E. Odell, M. Robinson, Evaluation of human papillomavirus testing for squamous cell carcinoma of the tonsil in clinical practice. J Clin Pathol. 64, 308-312 (2011).
54. I. H. Frazer, J. Chandra, Immunotherapy for HPV associated cancer. Papillomavirus Res. 8, 100176 (2019).
55. H. Wang, Q. Zhao, Y. Zhang, Q. Zhang, Z. Zheng, S. Liu, Z. Liu, L. Meng, Y. Xin, X. Jiang, Immunotherapy Advances in Locally Advanced and Recurrent/Metastatic Head and Neck Squamous Cell Carcinoma and Its Relationship With Human Papillomavirus. Front Immunol. 12, 652054 (2021).
56. H. Shibata, S. Saito, R. Uppaluri, Immunotherapy for Head and Neck Cancer: A Paradigm Shift From Induction Chemotherapy to Neoadjuvant Immunotherapy. Frontiers Oncol. 11, 727433 (2021).
57. A. T. Ruffin, A. R. Cillo, T. Tabib, A. Liu, S. Onkar, S. R. Kunning, C. Lampenfeld, H. I. Atiya, I. Abecassis, C. H. L. Kürten, Z. Qi, R. Soose, U. Duvvuri, S. Kim, S. Oesterrich, R. Lafyatis, L. G. Coffman, R. L. Ferris, D. A. A. Vignali, T. C. Bruno, B cell signatures and tertiary lymphoid structures contribute to outcome in head and neck squamous cell carcinoma. Nat Commun. 12, 3349 (2021).
58. M. H. Kim, J.-H. Kim, J. M. Lee, J. W. Choi, D. Jung, H. Cho, H. Kang, M. H. Hong, S. J. Heo, S. H. Kim, E. C. Choi, D. H. Kim, Y. M. Park, S. Kim, S. O. Yoon, Y. W. Koh, B. C. Cho, H. R. Kim, Molecular subtypes of oropharyngeal cancer show distinct immune microenvironment related with immune checkpoint blockade response. Brit J Cancer. 122, 1649-1660 (2020).
59. Y. Wang, Y. Xu, Q. Hua, Y. Jiang, P. Liu, W. Zhang, R. Xiang, Novel Prognostic Model Based on Immune Signature for Head and Neck Squamous Cell Carcinoma. Biomed Res Int. 2020, 1-9 (2020).
60. P. Charoentong, F. Finotello, M. Angelova, C. Mayer, M. Efremova, D. Rieder, H. Hackl, Z. Trajanoski, Pan-cancer Immunogenomic Analyses Reveal Genotype-Immunophenotype Relationships and Predictors of Response to Checkpoint Blockade. Cell Reports. 18, 248-262 (2017).
61. M. Liu, F. Li, B. Liu, Y. Jian, D. Zhang, H. Zhou, Y. Wang, Z. Xu, Profiles of immune cell infiltration and immune-related genes in the tumor microenvironment of esophageal squamous cell carcinoma. Bmc Med Genomics. 14, 2163-2180 (2021).
62. G. Bindea, B. Mlecnik, M. Tosolini, A. Kirilovsky, M. Waldner, A. C. Obenauf, H. Angell, T. Fredriksen, L. Lafontaine, A. Berger, P. Bruneval, W. H. Fridman, C. Becker, F. Pages, M. R. Speicher, Z. Trajanoski, J. Galon, Spatiotemporal Dynamics of Intratumoral Immune Cells Reveal the Immune Landscape in Human Cancer. Immunity. 39, 782-795 (2013).
63. S. V. Puram, I. Tirosh, A. S. Parikh, A. P. Patel, K. Yizhak, S. Gillespie, C. Rodman, C. L. Luo, E. A. Mroz, K. S. Emerick, D. G. Deschler, M. A. Varvares, R. Mylvaganam, O. Rozenblatt-Rosen, J. W. Rocco, W. C. Faquin, D. T. Lin, A. Regev, B. E. Bernstein, Single-Cell Transcriptomic Analysis of Primary and Metastatic Tumor Ecosystems in Head and Neck Cancer. Cell. 171, 1611-1624.e24 (2017).
64. D. Zhou, J. Wang, J. Wang, X. Liu, Profiles of immune cell infiltration and immune-related genes in the tumor microenvironment of HNSCC with or without HPV infection. Am J Transl Res. 4, 2163-2180 (2021).
65. F. Succaria, P. Kvistborg, J. E. Stein, E. L. Engle, T. L. McMiller, L. M. Rooper, E. Thompson, A. E. Berger, M. van den Brekel, C. L. Zuur, J. Haanen, S. L. Topalian, J. M. Taube, Characterization of the tumor immune microenvironment in human papillomavirus-positive and -negative head and neck squamous cell carcinomas. Cancer Immunol Immunother. 70, 1227-1237 (2020).
66. Z. Zhang, Y. Bao, L. Zhou, Y. Ye, W. Fu, C. Sun, DOCK8 Serves as a Prognostic Biomarker and Is Related to Immune Infiltration in Patients With HPV Positive Head and Neck Squamous Cell Carcinoma. Cancer Control. 28, 107327482110119 (2021).
67. Y. Chen, J. Nie, X. Li, T. Fan, X. Deng, D. Liang, G. Song, Identification of Immune-Related Prognostic Biomarkers Associated with HPV-Positive Head and Neck Squamous Cell Carcinoma. J Immunol Res. 2021, 1-23 (2021).
68. A. R. Cillo, C. H. L. Kürten, T. Tabib, Z. Qi, S. Onkar, T. Wang, A. Liu, U. Duvvuri, S. Kim, R. J. Soose, S. Oesterreich, W. Chen, R. Lafyatis, T. C. Bruno, R. L. Ferris, D. A. A. Vignali, Immune Landscape of Viral- and Carcinogen-Driven Head and Neck Cancer. Immunity. 52, 183-199.e9 (2020).
69. D. Kedra, H. Q. Pan, E. Seroussi, I. Fransson, C. Guilbaud, J. E. Collins, I. Dunham, E. Blennow, B. A. Roe, F. Piehl, J. P. Dumanski, Characterization of the human synaptogyrin gene family. Hum Genet. 103, 131-141 (1998).
70. R. Belizaire, C. Komanduri, K. Wooten, M. Chen, C. Thaller, R. Janz, Characterization of Synaptogyrin 3 as a New Synaptic Vesicle Protein. J Comp Neurol. 470, 266-281 (2004).
71. M. I. Love, W. Huber, S. Anders, Moderated estimation of fold change and dispersion for RNA-seq data with DESeq2. Genome Biol. 15, 550 (2014).
72. Z. Gu, R. Eils, M. Schlesner, Complex heatmaps reveal patterns and correlations in multidimensional genomic data. Bioinformatics. 32, 2847-2849 (2016).
73. A. Porrello, P. L. Leslie, E. B. Harrison, B. K. Gorentla, S. Kattula, S. K. Ghosh, S. H. Azam, A. Holtzhausen, Y. L. Chao, M. C. Hayward, T. A. Waugh, S. Bae, V. Godfrey, S. H. Randell, C. Oderup, L. Makowski, J. Weiss, M. D. Wilkerson, D. N. Hayes, H. S. Earp, A. S. Baldwin, A. S. Wolberg, C. V. Pecot, Factor XIIIA-expressing inflammatory monocytes promote lung squamous cancer through fibrin cross-linking. Nat Commun. 9, 1988 (2018).
74. Y. Hao, S. Hao, E. Andersen-Nissen, W. M. Mauck, S. Zheng, A. Butler, M. J. Lee, A. J. Wilk, C. Darby, M. Zager, P. Hoffman, M. Stoeckius, E. Papalexi, E. P. Mimitou, J. Jain, A. Srivastava, T. Stuart, L. M. Fleming, B. Yeung, A. J. Rogers, J. M. McElrath, C. A. Blish, R. Gottardo, P. Smibert, R. Satija, Integrated analysis of multimodal single-cell data. Cell. 184, 3573-3587.e29 (2021).
75. D. Aran, A. P. Looney, L. Liu, E. Wu, V. Fong, A. Hsu, S. Chak, R. P. Naikawadi, P. J. Wolters, A. R. Abate, A. J. Butte, M. Bhattacharya, Reference-based analysis of lung single-cell sequencing reveals a transitional profibrotic macrophage. Nat Immunol. 20, 163-172 (2019).
76. G. Monaco, B. Lee, W. Xu, S. Mustafah, Y. Y. Hwang, C. Cane, N. Burdin, L. Visan, M. Ceccarelli, M. Poidinger, A. Zippelius, J. P. de Magalhães, A. Larbi, RNA-Seq Signatures Normalized by mRNA Abundance Allow Absolute Deconvolution of Human Immune Cell Types. Cell Reports. 26, 1627-1640.e7 (2019).
77. M. B. Carper, S. Troutman, B. L. Wagner, K. M. Byrd, S. R. Selitsky, K. Parag-Sharma, E. C. Henry, W. Li, J. S. Parker, S. A. Montgomery, J. L. Cleveland, S. E. Williams, J. L. Kissil, D. N. Hayes, A. L. Amelio, An Immunocompetent Mouse Model of HPV16(+) Head and Neck Squamous Cell Carcinoma. Cell Reports. 29, 1660-1674.e7 (2019).
78. K. Divans, A. F. Olshan, J. Smith, M. E. Bell, M. C. Weissler, W. K. Funkhouser, P. T. Bradshaw, Oral health and risk for head and neck squamous cell carcinoma: The Carolina Head and Neck Cancer Study. Cancer Cause Control. 21, 567-575 (2010).
79. N. Zhao, M. K. Ang, X. Y. Yin, M. R. Patel, K. Fritchie, L. Thorne, K. L. Muldrew, M. C. Hayward, W. Sun, M. D. Wilkerson, B. S. Chera, T. Hackman, A. M. Zanation, J. E. Grilley-Olson, M. E. Couch, W. W. Shockley, M. C. Weissler, C. G. Shores, W. K. Funkhouser, A. F. Olshan, D. N. Hayes, Different cellular p16 INK4a localisation may signal different survival outcomes in head and neck cancer. Brit J Cancer. 107, 482-490 (2012).
80. X. Robin, N. Turck, A. Hainard, N. Tiberti, F. Lisacek, J.-C. Sanchez, M. Muller, pROC: an open-source package for R and S+ to analyze and compare ROC curves. Bmc Bioinformatics. 12, 77 (2011).
81. B. S. Chera, S. Kumar, C. Shen, R. Amdur, R. Dagan, R. Green, E. Goldman, J. Weiss, J. Grilley-Olson, S. Patel, A. Zanation, T. Hackman, J. Blumberg, S. Patel, B. Thorp, M. Weissler, W. Yarbrough, N. Sheets, W. Mendenhall, X. M. Tan, G. P. Gupta, Plasma Circulating Tumor HPV DNA for the Surveillance of Cancer Recurrence in HPV-Associated Oropharyngeal Cancer. J Clin Oncol. 38, 1050-1058 (2020).
82. B. S. Chera, S. Kumar, B. T. Beaty, D. Marron, S. Jefferys, R. Green, E. C. Goldman, R. Amdur, N. Sheets, R. Dagan, D. N. Hayes, J. Weiss, J. E. Grilley-olson, A. Zanation, T. Hackman, J. M. Blumberg, S. Patel, M. Weissler, X. M. Tan, J. S. Parker, W. Mendenhall, G. P. Gupta, Rapid Clearance Profile of Plasma Circulating Tumor HPV Type 16 DNA during Chemoradiotherapy Correlates with Disease Control in HPV-Associated Oropharyngeal Cancer. Clin Cancer Res. 25, 4682-4691 (2019).
83. H. Reder, S. Wagner, N. Wuerdemann, C. Langer, S. Sandmann, A. Braeuninger, M. Dugas, S. Gattenloehner, C. Wittekindt, J. P. Klussmann, Mutation patterns in recurrent and/or metastatic oropharyngeal squamous cell carcinomas in relation to human papillomavirus status. Cancer Med-us. 10, 1347-1356 (2021).
84. P. Usha, M. Neha, R. U. Swapnil, P. Asawari, G. Poonam, K. Sadhana, J. Amit, N. Vanita, P. M. Vijay, P. Kumar, M. B. Manoj, Correlation of transcriptionally active human papillomavirus status with the clinical and molecular profiles of head and neck squamous cell carcinomas. Head Neck. 43, 2032-2044 (2021).
85. D. Camuzi, L. A. Buexm, S. de Q. C. Lourenco, D. D. Esposti, C. Cuenin, M. de S. A. Lopes, F. Manara, F. R. Talukdar, Z. Herceg, L. F. R. Pinto, S. C. Soares-Lima, HPV Infection Leaves a DNA Methylation Signature in Oropharyngeal Cancer Affecting Both Coding Genes and Transposable Elements. Cancers. 13, 3621 (2021).
86. P. Largo-Barrientos, N. Apostolo, E. Creemers, Z. Callaerts-Vegh, J. Swerts, C. Davies, J. McInnes, K. Wierda, B. D. Strooper, T. Spires-Jones, J. de Wit, V. Uytterhoeven, P. Verstreken, Lowering Synaptogyrin-3 expression rescues Tau-induced memory defects and synaptic loss in the presence of microglial activation. Neuron. 109, 1-11 (2021).
87. L. A. Egaña, R. A. Cuevas, T. B. Baust, L. A. Parra, R. K. Leak, S. Hochendoner, K. Pena, M. Quiroz, W. C. Hong, M. M. Dorostkar, R. Janz, H. H. Sitte, G. E. Tones, Physical and functional interaction between the dopamine transporter and the synaptic vesicle protein synaptogyrin-3. J Neurosci. 29, 4592-4604 (2009).
88. J. McInnes, K. Wierda, A. Snellinx, L. Bounti, Y. C. Wang, I. C. Stancu, N. Apostolo, K. Gevaert, I. Dewachter, T. L. Spires-Jones, B. D. Strooper, J. D. Wit, L. Zhou, P. Verstreken, Synaptogyrin-3 Mediates Presynaptic Dysfunction Induced by Tau. Neuron. 97, 823-835.e8 (2018).
89. K. Boesze-Battaglia, A. Dhingra, L. M. Walker, A. Zekavat, B. J. Shenker, Internalization and Intoxication of Human Macrophages by the Active Subunit of the Aggregatibacter actinomycetemcomitans Cytolethal Distending Toxin Is Dependent Upon Cellugyrin (Synaptogyrin-2). Front Immunol. 11, 1262 (2020).
90. C. Cochain, E. Vafadarnejad, P. Arampatzi, J. Pelisek, H. Winkels, K. Ley, D. Wolf, A.-E. Saliba, A. Zernecke, Single-Cell RNA-Seq Reveals the Transcriptional Landscape and Heterogeneity of Aortic Macrophages in Murine Atherosclerosis. Circ Res. 122, 1661-1674 (2018).
91. M. R. Patel, N. Zhao, M. K. Ang, M. E. Stadler, K. Fritchie, M. C. Weissler, A. M. Zanation, S. L. Harris, W. K. Funkhouser, A. F. Olshan, C. G. Shores, D. N. Hayes, ERCC1 protein expression is associated with differential survival in oropharyngeal head and neck squamous cell carcinoma. Otolaryngology Head Neck Surg. 149, 587-595 (2013).
92. B. Nguyen, K. Meehan, M. R. Pereira, B. Mirzai, S. H. Lim, C. Leslie, M. Clark, C. Sader, P. Friedland, A. Lindsay, C. Tang, M. Millward, E. S. Gray, A. M. Lim, A comparative study of extracellular vesicle-associated and cell-free DNA and RNA for HPV detection in oropharyngeal squamous cell carcinoma. Sci Rep-uk. 10, 1-10 (2020).
93. C. T. Haring, C. Bhambhani, C. Brummel, B. Jewell, E. Bellile, M. E. H. Neal, E. Sandford, R. M. Spengler, A. Bhangale, M. E. Spector, J. McHugh, M. E. Prince, M. Mierzwa, F. P. Worden, M. Tewari, P. L. Swiecicki, J. C. Brenner, Human papilloma virus circulating tumor DNA assay predicts treatment response in recurrent/metastatic head and neck squamous cell carcinoma. Oncotarget. 12, 1214-1229 (2021).
94. M. J. Goldman, B. Craft, M. Hastie, K. Repečka, F. McDade, A. Kamath, A. Banerjee, Y. Luo, D. Rogers, A. N. Brooks, J. Zhu, D. Haussler, Visualizing and interpreting cancer genomics data via the Xena platform. Nat Biotechnol. 38, 675-678 (2020).
95. S. Lai, A. E. Wenaas, V. C. Sandulache, C. Hartman, E. Chiao, J. Kramer, J. P. Zevallos, Prognostic Significance of p16 Cellular Localization in Oropharyngeal Squamous Cell Carcinoma. Annals of Clinical & Laboratory Science. 46, 132-139 (2016).
96. O. Adeniyi, F. Aguel, A. Agyeman, S. Amur, K. Bouri, D. Canos, K. Capanna, A. G. Chakravarty, D. S. Cho, S. Fitzpatrick, I. Irony, D. Krainak, E. L. Kunkoski, S. Lababidi, C. Leptak, W. Mattes, E. Mishina, M. A. Pacanowski, E. J. Papadopoulos, V. Peiris, R. Puri, J. Ruiz, R. J. Temple, P. Turfle, S. J. Wang, B. Zhang, T. G. Lively, L. M. McShane, BEST (Biomarkers, EndpointS, and other Tools) Resource [Internet]. (Silver Spring (Md.): Food and Drug Administration (US); Bethesda (Md.): National Institutes of Health (US), 2021).
97. A. J. Atkinson, W. G. Magnuson, W. A. Colburn, V. G. DeGruttola, D. L. DeMets, G. J. Downing, D. F. Hoth, J. A. Oates, C. C. Peck, R. T. Schooley, B. A. Spilker, J. Woodcock, S. L. Zeger, Biomarkers and surrogate endpoints: Preferred definitions and conceptual framework. Clin Pharmacol Ther. 69, 89-95 (2001).
98. N. L. Henry, D. F. Hayes, Cancer biomarkers. Mol Oncol. 6, 140-146 (2012).
99. S. M. Teutsch, L. A. Bradley, G. E. Palomaki, J. E. Haddow, M. Piper, N. Calonge, W. D. Dotson, M. P. Douglas, A. O. Berg, E. W. Group, The Evaluation of Genomic Applications in Practice and Prevention (EGAPP) initiative: methods of the EGAPP Working Group. Genet Med. 11, 3-14 (2009).
100. H. M. Moore, A. B. Kelly, S. D. Jewell, L. M. McShane, D. P. Clark, R. Greenspan, D. F. Hayes, P. Hainaut, P. Kim, E. A. Mansfield, O. Potapova, P. Riegman, Y. Rubinstein, E. Seijo, S. Somiari, P. Watson, H. Weier, C. Zhu, J. Vaught, Biospecimen reporting for improved study quality (BRISQ). Cancer Cytopathol. 119, 92-102 (2011).
101. D. F. Ransohoff, How to improve reliability and efficiency of research about molecular markers: roles of phases, guidelines, and study design. J Clin Epidemiol. 60, 1205-1219 (2007).
102. R. M. Simon, S. Paik, D. F. Hayes, Use of Archived Specimens in Evaluation of Prognostic and Predictive Biomarkers. Jnci J National Cancer Inst. 101, 1446-1452 (2009).
103. W. Sauerbrei, S. E. Taube, L. M. McShane, M. M. Cavenagh, D. G. Altman, Reporting Recommendations for Tumor Marker Prognostic Studies (REMARK): An Abridged Explanation and Elaboration. Jnci J National Cancer Inst. 110, 803-811 (2018).
104. P. M. Bossuyt, J. B. Reitsma, D. E. Bruns, C. A. Gatsonis, P. P. Glasziou, L. M. Irwig, D. Moher, D. Rennie, H. C. W. de Vet, J. G. Lijmer, The STARD Statement for Reporting Studies of Diagnostic Accuracy: Explanation and Elaboration. Ann Intern Med. 138, W1 (2003).
105. C. F. Taylor, N. W. Paton, K. S. Lilley, P.-A. Binz, R. K. Julian, A. R. Jones, W. Zhu, R. Apweiler, R. Aebersold, E. W. Deutsch, M. J. Dunn, A. J. R. Heck, A. Leitner, M. Macht, M. Mann, L. Martens, T. A. Neubert, S. D. Patterson, P. Ping, S. L. Seymour, P. Souda, A. Tsugita, J. Vandekerckhove, T. M. Vondriska, J. P. Whitelegge, M. R. Wilkins, I. Xenarios, J. R. Yates, H. Hermjakob, The minimum information about a proteomics experiment (MIAPE). Nat Biotechnol. 25, 887-893 (2007).
106. D. F. Hayes, R. C. Bast, C. E. Desch, H. Fritsche, N. E. Kemeny, J. M. Jessup, G. Y. Locker, J. S. Macdonald, R. G. Mennel, L. Norton, P. Ravdin, S. Taube, R. J. Winn, Tumor Marker Utility Grading System: a Framework to Evaluate Clinical Utility of Tumor Markers. Jnci J National Cancer Inst. 88, 1456-1466 (1996).
107. R. Abgral, S. Querellou, G. Potard, P.-Y. L. Roux, A. L. Duc-Pennec, R. Marianovski, O. Pradier, Y. Bizais, F. Kraeber-Bodéré, P. Y. Salaun, Does 18F-FDG PET/CT Improve the Detection of Posttreatment Recurrence of Head and Neck Squamous Cell Carcinoma in Patients Negative for Disease on Clinical Follow-up? J Nucl Med. 50, 24-29 (2009).
108. M. G. Isles, C. McConkey, H. M. Mehanna, A systematic review and meta-analysis of the role of positron emission tomography in the follow up of head and neck squamous cell carcinoma following radiotherapy or chemoradiotherapy. Clin Otolaryngol. 33, 210-222 (2008).
109. P. B. Romesser, M. M. Qureshi, B. A. Shah, L. T. Chatburn, S. Jalisi, A. K. Devaiah, R. M. Subramaniam, M. T. Truong, Superior prognostic utility of gross and metabolic tumor volume compared to standardized uptake value using PET/CT in head and neck squamous cell carcinoma patients treated with intensity-modulated radiotherapy. Ann Nucl Med. 26, 527-534 (2012).
110. J. E. Leeman, N. Katabi, R. J. Wong, N. Y. Lee, P. B. Romesser, Abeloff's Clinical Oncology, 999-1033.e7 (2020).
111. S. K. Mukherji, S. M. O'Brien, R. J. Gerstle, M. Weissler, W. Shockley, J. A. Stone, M. Castillo, The ability of tumor volume to predict local control in surgically treated squamous cell carcinoma of the supraglottic larynx. Head Neck. 22, 282-287 (2000).
112. W. M. Mendenhall, J. T. Parsons, A. A. Mancuso, F. J. Pameijer, S. P. Stringer, N. J. Cassisi, Definitive radiotherapy for T3 squamous cell carcinoma of the glottic larynx. J Clin Oncol. 15, 2394-2402 (1997).
113. S. K. Mukherji, A. A. Mancuso, W. Mendenhall, I. M. Kotzur, P. Kubilis, Can Pretreatment CT Predict Local Control of T2 Glottic Carcinomas Treated with Radiation Therapy Alone? AJNR. 16, 655-662 (1995).
114. W. R. Lee, A. A. Mancuso, E. M. Saleh, W. M. Mendenhall, J. T. Parsons, R. R. Million, Can pretreatment computed tomography findings predict local control in T3 squamous cell carcinoma of the glottic larynx treated with radiotherapy alone? Int J Radiat Oncol Biology Phys. 25, 683-687 (1993).
115. I. Doweck, D. Denys, K. T. Robbins, Tumor Volume Predicts Outcome for Advanced Head and Neck Cancer Treated With Targeted Chemoradiotherapy. Laryngoscope. 112, 1742-1749 (2002).
116. G. Studer, U. M. Lütolf, M. El-Bassiouni, V. Rousson, C. Glanzmann, Volumetric staging (VS) is superior to TNM and AJCC staging in predicting outcome of head and neck cancer treated with IMRT. Acta Oncol. 46, 386-394 (2009).
117. A. Strongin, S. Yovino, R. Taylor, J. Wolf, K. Cullen, A. Zimrin, S. Strome, W. Regine, M. Suntharalingam, Primary Tumor Volume Is an Important Predictor of Clinical Outcomes Among Patients With Locally Advanced Squamous Cell Cancer of the Head and Neck Treated With Definitive Chemoradiotherapy. Int JRadiat Oncol Biology Phys. 82, 1823-1830 (2012).
118. B. H. Lok, J. Setton, N. Caria, J. Romanyshyn, S. L. Wolden, M. J. Zelefsky, J. Park, N. Rowan, E. J. Sherman, M. G. Fury, A. Ho, D. G. Pfister, R. J. Wong, J. P. Shah, D. H. Kraus, Z. Zhang, K. D. Schupak, D. Y. Gelblum, S. D. Rao, N. Y. Lee, Intensity-Modulated Radiation Therapy in Oropharyngeal Carcinoma: Effect of Tumor Volume on Clinical Outcomes. Int J Radiat Oncol Biology Phys. 82, 1851-1857 (2012).
119. M. Chen, T. H. Chen, J. Liu, C. Chang, W. Chie, Better prediction of prognosis for patients with nasopharyngeal carcinoma using primary tumor volume. Cancer. 100, 2160-2166 (2004).
120. T. H. La, E. J. Filion, B. B. Turnbull, J. N. Chu, P. Lee, K. Nguyen, P. Maxim, A. Quon, E. E. Graves, B. W. Loo, Q.-T. Le, Metabolic Tumor Volume Predicts for Recurrence and Death in Head-and-Neck Cancer. Int J Radiat Oncol Biology Phys. 74, 1335-1341 (2009).
121. K. P. Chu, J. D. Murphy, T. H. La, T. E. Krakow, A. Iagaru, E. E. Graves, A. Hsu, P. G. Maxim, B. Loo, D. T. Chang, Q.-T. Le, Prognostic Value of Metabolic Tumor Volume and Velocity in Predicting Head-and-Neck Cancer Outcomes. Int J Radiat Oncol Biology Phys. 83, 1521-1527 (2012).
122. J. P. Kühn, W. Schmid, S. Kömer, F. Bochen, S. Wemmert, H. Rimbach, S. Smola, J. C. Radosa, M. Wagner, L. G. T. Morris, V. Bozzato, A. Bozzato, B. Schick, M. Linxweiler, HPV Status as Prognostic Biomarker in Head and Neck Cancer—Which Method Fits the Best for Outcome Prediction? Cancers. 13, 4730 (2021).
123. L. Masterson, D. Moualed, Z. W. Liu, J. E. F. Howard, R. C. Dwivedi, J. R. Tysome, R. Benson, J. C. Sterling, H. Sudhoff, P. Jani, P. K. C. Goon, De-escalation treatment protocols for human papillomavirus-associated oropharyngeal squamous cell carcinoma: A systematic review and meta-analysis of current clinical trials. Eur J Cancer. 50, 2636-2648 (2014).
124. F. O. Gleber-Netto, X. Rao, T. Guo, Y. Xi, M. Gao, L. Shen, K. Erikson, N. N. Kalu, S. Ren, G. Xu, K. M. Fisch, K. Akagi, T. Seiwert, M. Gillison, M. J. Frederick, F. M. Johnson, J. Wang, J. N. Myers, J. Califano, H. D. Skinner, C. R. Pickering, Variations in HPV function are associated with survival in squamous cell carcinoma. Jci Insight. 4, e124762 (2019).
125. J. Lei, A. Ploner, C. Lagheden, C. Eklund, S. N. Kleppe, B. Andrae, K. M. Elfström, J. Dillner, P. Sparén, K. Sundström, Human papillomavirus genotype and prognosis of invasive cervical cancer: A nationwide cohort study. J Clin Oncol. 37, 5525-5525 (2019).
126. R. A. Harbison, M. Kubik, E. Q. Konnick, Q. Zhang, S.-G. Lee, H. Park, J. Zhang, C. S. Carlson, C. Chen, S. M. Schwartz, C. P. Rodriguez, U. Duvvuri, E. Méndez, The mutational landscape of recurrent versus nonrecurrent human papillomavirus—related oropharyngeal cancer. Jci Insight. 3, e99327 (2018).
127. H. Reder, S. Wagner, U. Gamerdinger, S. Sandmann, N. Wuerdemann, A. Braeuninger, M. Dugas, S. Gattenloehner, J. P. Klussmann, C. Wittekindt, Genetic alterations in human papillomavirus-associated oropharyngeal squamous cell carcinoma of patients with treatment failure. Oral Oncol. 93, 59-65 (2019).
128. M. Oberste, A. Riders, B. Abbaspour, L. Kerschke, A. G. Beule, C. Rudack, Improvement of patient stratification in human papilloma virus-associated oropharyngeal squamous cell carcinoma by defining a multivariable risk score. Head Neck. 43, 3314-3323 (2021).
129. K. M. V. Abel, D. M. Routman, E. J. Moore, D. J. Ma, L. X. Yin, P. A. Fields, M. Schofield, K. R. Bartemes, K. Chatzopoulos, D. L. Price, J. R. Janus, J. L. Kasperbauer, K. A. Price, A. V. Chintakuntlawar, M. A. Neben-Wittich, R. L. Foote, J. J. Garcia, T cell fraction impacts oncologic outcomes in human papillomavirus associated oropharyngeal squamous cell carcinoma. Oral Oncol. 111, 104894 (2020).
130. C. S. Eberhardt, H. T. Kissick, M. R. Patel, M. A. Cardenas, N. Prokhnevska, R. C. Obeng, T. H. Nasti, C. C. Griffith, S. J. Im, X. Wang, D. M. Shin, M. Carrington, Z. G. Chen, J. Sidney, A. Sette, N. F. Saba, A. Wieland, R. Ahmed, Functional HPV-specific PD-1+ stem-like CD8 T cells in head and neck cancer. Nature. 597, 279-284 (2021).
131. J. R. Lechien, I. Seminerio, G. Descamps, Q. Mat, F. Mouawad, S. Hans, M. Julieron, D. Dequanter, T. Vanderhaegen, F. Journe, S. Saussez, Impact of HPV Infection on the Immune System in Oropharyngeal and Non-Oropharyngeal Squamous Cell Carcinoma: A Systematic Review. Cells. 8, 1061 (2019).
132. L. Vanhersecke, M. Brunet, J.-P. Guégan, C. Rey, A. Bougouin, S. Cousin, S. L. Moulec, B. Besse, Y. Loriot, M. Larroquette, I. Soubeyran, M. Toulmonde, G. Roubaud, S. Perrot, M. Cohort, F. Chomy, C. Lefevre, K. Bourcier, M. Kind, I. Giglioli, C. Sautès-Fridman, V. Velasco, F. Courgeon, E. Oflazoglu, A. Savina, A. Marabelle, J.-C. Soria, C. Bellera, C. Sofeu, A. Bessede, W. H. Fridman, F. L. Loarer, A. Italiano, Mature tertiary lymphoid structures predict immune checkpoint inhibitor efficacy in solid tumors independently of PD-L1 expression. Nat Cancer. 2, 794-802 (2021).
133. J. Goc, C. Germain, T. K. D. Vo-Bourgais, A. Lupo, C. Klein, S. Knockaert, L. de Chaisemartin, H. Ouakrim, E. Becht, M. Alifano, P. Validire, R. Remark, S. A. Hammond, I. Cremer, D. Damotte, W.-H. Fridman, C. Sautès-Fridman, M.-C. Dieu-Nosjean, Dendritic Cells in Tumor-Associated Tertiary Lymphoid Structures Signal a Th1 Cytotoxic Immune Contexture and License the Positive Prognostic Value of Infiltrating CD8+ T Cells. Cancer Res. 74, 705-715 (2014).
134. C. Sautès-Fridman, F. Petitprez, J. Calderaro, W. H. Fridman, Tertiary lymphoid structures in the era of cancer immunotherapy. Nat Rev Cancer. 19, 307-325 (2019).
135. K. M. Smith, L. Pottage, E. R. Thomas, A. J. Leishman, T. N. Doig, D. Xu, F. Y. Liew, P. Garside, Th1 and Th2 CD4+ T Cells Provide Help for B Cell Clonal Expansion and Antibody Synthesis in a Similar Manner In Vivo. J Immunol. 165, 3136-3144 (2000).
136. Y. Su, Z. Zeng, D. Rong, Y. Yang, B. Wu, Y. Cao, PSMC2, ORCS and KRTDAP are specific biomarkers for HPV-negative head and neck squamous cell carcinoma. Oncol Lett. 21, 289 (2021).
137. H. Chang, H. C. Jeung, J. J. Jung, T. S. Kim, S. Y. Rha, H. C. Chung, Identification of genes associated with chemosensitivity to SAHA/taxane combination treatment in taxane-resistant breast cancer cells. Breast Cancer Res Tr. 125, 55-63 (2011).
138. G. Lopes-Santos, D. G. Bernabé G. I. Miyahara, K. C. Tjioe, Beta-adrenergic pathway activation enhances aggressiveness and inhibits sternness in head and neck cancer. Transl Oncol. 14, 101117 (2021).
139. S. W. Cole, A. K. Sood, Molecular Pathways: Beta-Adrenergic Signaling in Cancer. Clin Cancer Res. 18, 1201-1206 (2012).
140. M. H. Tan, C. F. Wong, H. L. Tan, X. J. Yang, J. Ditlev, D. Matsuda, S. K. Khoo, J. Sugimura, T. Fujioka, K. A. Furge, E. Kort, S. Giraud, S. Ferlicot, P. Vielh, D. Amsellem-Ouazana, B. Debré, T. Flam, N. Thiounn, M. Zerbib, G. Beno\hat{ }it, S. Droupy, V. Molini\'e, A. Vieillefond, P. H. Tan, S. Richard, B. T. Teh, Genomic expression and single-nucleotide polymorphism profiling discriminates chromophobe renal cell carcinoma and oncocytoma. Bmc Cancer. 10, 1-12 (2010).
141. S. Ludwig, P. Sharma, M.-N. Theodoraki, M. Pietrowska, S. S. Yemeni, S. Lang, S. Ferrone, T. L. Whiteside, Molecular and Functional Profiles of Exosomes From HPV(+) and HPV(−) Head and Neck Cancer Cell Lines. Frontiers Oncol. 8, 445 (2018).
142. C. Goudsmit, F. da V. Leprevost, V. Basrur, L. Peters, A. Nesvizhskii, H. Walline, Differences in Extracellular Vesicle Protein Cargo Are Dependent on Head and Neck Squamous Cell Carcinoma Cell of Origin and Human Papillomavirus Status. Cancers. 13, 3714 (2021).
143. K. Luck, D. K. Kim, L. Lambourne, K. Spirohn, B. E. Begg, W. Bian, R. Brignall, T. Cafarelli, F. J. Campos-Laborie, B. Charloteaux, D. Choi, A. G. Cote, M. Daley, S. Deimling, A. Desbuleux, A. Dricot, M. Gebbia, M. F. Hardy, N. Kishore, J. J. Knapp, I. A. Kovacs, I. Lemmens, M. W. Mee, J. C. Mellor, C. Pollis, C. Pons, A. D. Richardson, S. Schlabach, B. Teeking, A. Yadav, M. Babor, D. Balcha, O. Basha, C. Bowman-Colin, S. F. Chin, S. G. Choi, C. Colabella, G. Coppin, C. D'Amata, D. D. Ridder, S. D. Rouck, M. Duran-Frigola, H. Ennajdaoui, F. Goebels, L. Goehring, A. Gopal, G. Haddad, E. Hatchi, M. Helmy, Y. Jacob, Y. Kassa, S. Landini, R. Li, N. van Lieshout, A. MacWilliams, D. Markey, J. N. Paulson, S. Rangarajan, J. Rasla, A. Rayhan, T. Rolland, A. San-Miguel, Y. Shen, D. Sheykhkarimli, G. M. Sheynkman, E. Simonovsky, M. Taşan, A. Tejeda, V. Tropepe, J. C. Twizere, Y. Wang, R. J. Weatheritt, J. Weile, Y. Xia, X. Yang, E. Yeger-Lotem, Q. Zhong, P. Aloy, G. D. Bader, J. D. L. Rivas, S. Gaudet, T. Hao, J. Rak, J. Tavernier, D. E. Hill, M. Vidal, F. P. Roth, M. A. Calderwood, A reference map of the human binary protein interactome. Nature. 580, 402-408 (2020).
144. M. Hajek, A. Sewell, S. Kaech, B. Burtness, W. G. Yarbrough, N. Issaeva, TRAF3/CYLD mutations identify a distinct subset of human papillomavirus-associated head and neck squamous cell carcinoma. Cancer. 123, 1778-1790 (2017).
145. M. B. Koenigs, A. Lefranc-Torres, J. Bonilla-Velez, K. B. Patel, D. N. Hayes, K. Glomski, P. M. Busse, A. W. Chan, J. R. Clark, D. G. Deschler, K. S. Emerick, R. J. Hammon, L. J. Wirth, D. T. Lin, E. A. Mroz, W. C. Faquin, J. W. Rocco, Association of Estrogen Receptor Alpha Expression With Survival in Oropharyngeal Cancer Following Chemoradiation Therapy. Jnci J National Cancer Inst. 111, 933-942 (2019).
146. J. R. Shinn, S. J. Davis, K. A. Lang-Kuhs, S. Rohde, X. Wang, P. Liu, W. D. Dupont, D. Plummer, W. L. Thorstad, R. D. Chernock, M. Mehrad, J. S. Lewis, Oropharyngeal Squamous Cell Carcinoma With Discordant p16 and HPV mRNA Results. Am J Surg Pathology. 45, 951-961 (2021).
147. V. Ramshankar, V. T. Soundara, V. Shyamsundar, P. Ramani, A. Krishnamurthy, Risk Stratification of Early Stage Oral Tongue Cancers Based on HPV Status and p16 Immunoexpression. Asian Pac J Cancer P. 15, 8351-8359 (2014).
148. A. M. Lim, H. Do, R. J. Young, S. Q. Wong, C. Angel, M. Collins, E. A. Takano, J. Corry, D. Wiesenfeld, S. Kleid, E. Sigston, B. Lyons, S. B. Fox, D. Rischin, A. Dobrovic, B. Solomon, Differential mechanisms of CDKN2A (p16) alteration in oral tongue squamous cell carcinomas and correlation with patient outcome. Int J Cancer. 135, 887-895 (2014).
149. K. Schneider, K. K. Jakobsen, J. S. Jensen, I. Wessel, A. Christensen, L. Specht, G. Lelkaitis, C. Mirian, C. v. Buchwald, C. Gronhoj, Impact of p16-overexpression on overall and progression-free survival outcomes in oral cavity squamous cell carcinomas: A semi-national, population-based study. Oral Oncol. 111, 105031 (2020).
150. C. Bouland, D. Dequanter, J. R. Lechien, C. Hanssens, N. D. S. Aubain, A. Digonnet, R. Javadian, A. Yanni, A. Rodriguez, I. Loeb, F. Journe, S. Saussez, Prognostic Significance of a Scoring System Combining p16, Smoking, and Drinking Status in a Series of 131 Patients with Oropharyngeal Cancers. Int J Otolaryngology. 2021, 1-6 (2021).
151. F. F. Brkic, C. Mayer, G. Besser, G. Altorjai, H. Herrmann, G. Heiduschka, G. Haymerle, L. Kadletz-Wanke, Potential association of the prognostic index and survival in patients with p16-positive oropharyngeal squamous cell carcinoma. Wien Klin Wochenschr, 1-5 (2021).
152. M. Wendt, L. Hammarstedt-Nordenvall, M. Zupancic, S. Friesland, D. Landin, E. Munck-Wikland, T. Dalianis, A. Näsman, L. Marklund, Long-Term Survival and Recurrence in Oropharyngeal Squamous Cell Carcinoma in Relation to Subsites, HPV, and p16-Status. Cancers. 13, 2553 (2021).

All publications, patent application, patents and other references cited herein are incorporated by reference in their entireties for the teachings relevant to the sentence and/or paragraph in which the reference is presented.

The foregoing is illustrative of the present invention, and is not to be construed as limiting thereof. The invention is defined by the following claims, with equivalents of the claims to be included therein.

What is claimed is:

1. A method of diagnosing and treating head and neck squamous cell carcinoma (HNSCC) in a subject, comprising:
a) obtaining a sample from the subject comprising one or more immune cells;
b) detecting a level of expression of a SYNGR3 gene and/or gene product in the one or more immune cells;
c) comparing the level of expression detected in (b) with a level of expression of the SYNGR3 gene and/or gene product from a sample comprising one or more immune cells obtained from a control subject;
d) diagnosing the subject as being positive for human papillomavirus (HPV) when the level of expression of the SYNGR3 gene and/or gene product is greater than the level of expression of the SYNGR3 gene product of the control subject; and
e) treating the HNSCC in the subject with a protocol for patients with that are infected by HPV-positive HNSCC.

2. The method of claim 1, wherein the sample is from a HNSCC tumor in the subject.

3. The method of claim 1, wherein the gene product comprises one or more of a mRNA and a protein.

4. The method of claim 1, further comprising the steps of:
detecting a level of expression of a p16 gene and/or gene product in the sample, and
comparing a level of p16 gene and/or gene product in the sample with the level of expression of a p16 gene and/or gene product from the control subject;
wherein the subject is diagnosed as positive for HPV when the level of expression of the SYNGR3 gene and/or gene product is greater than the level of expression of the SYNGR3 gene and/or gene product of the control subject and the level of the p16 gene and/or gene product is greater than the level of expression of the p16 gene and/or gene product of the control subject.

5. The method of claim 1, wherein the detecting step comprises performing in situ hybridization (ISH) and/or immunohistochemistry (IHC) of SYNGR3 and/or p16 gene and/or gene product on the sample of the subject and/or the control subject.

6. The method of claim 1, wherein the comparing step comprises quantifying the gene and/or gene product expression level, comparing the quantified gene and/or gene product expression level between that of the subject and that of the control subject, and optionally, generating a ROC curve.

7. The method of claim 1, further comprising determining an HPV status for the subject.

8. A method of diagnosing and treating head and neck squamous cell carcinoma (HNSCC) in a subject, comprising treating the HNSCC in the subject with a protocol for patients with HPV-positive HNSCC if the subject is diagnosed as having an overexpression of SYNGR3 gene and/or gene product in one or more immune cells in a HNSCC tumor of the subject.

9. The method of claim 8, wherein the HNSCC in the subject is treated with a protocol for patients with HPV-positive HNSCC if the subject is diagnosed as having the overexpression of SYNGR3 gene and/or gene product in the one or more immune cells in the HNSCC tumor and an overexpression of p16 gene and/or gene product in the HNSCC tumor, and/or if the subject is further diagnosed as being HPV-positive based on HPV16 in situ hybridization (ISH).

10. A method of diagnosing and treating head and neck squamous cell carcinoma (HNSCC) in a subject comprising:
determining a SYNGR3 status of the subject based on a level of expression of the SYNGR3 gene and/or gene product in one or more immune cells in a tumor of the subject;
determining a p16 status of the subject based on a level of expression of a p16 gene and/or gene product in the tumor of the subject;
diagnosing the subject has having HPV-positive HNSCC based on the SYNGR3 status and the p16 status of the subject; and
if the subject is diagnosed as having HPV-positive HNSCC, treating the HNSCC in the subject with a protocol for patients with that have HPV-positive HNSCC.

11. The method of claim 10, further comprising determining an ISH status of the subject by performing HPV16 in situ hybridization (ISH) and diagnosing the subject has having HPV-positive HNSCC based on the SYNGR3 status, the p16 status of the subject, and the ISH status of the subject.

12. The method of claim 10, wherein the SYNGR3 status is determined based on a level of overexpression of the SYNGR3 gene and/or gene product.

13. The method of claim 12, wherein the level of overexpression of the SYNGR3 gene and/or gene product is determined by quantifying the SYNGR3 gene and/or gene product expression level, comparing the gene and/or gene product expression level quantified for the subject and quantified for a control subject, and optionally, generating a ROC curve.

14. The method of claim 13, wherein the control subject is a reference sample and/or a reference level as established in a reference database.

15. A method of diagnosing and treating head and neck squamous cell carcinoma (HNSCC) in a subject, comprising:
a) obtaining a sample from the subject comprising one or more immune cells;
b) detecting a level of expression of a SYNGR3 gene and/or gene product in the one or more immune cells;
c) comparing the level of expression detected in (b) with a reference level of expression of the SYNGR3 gene and/or gene product;
d) diagnosing the subject as being positive for human papillomavirus (HPV) when the level of expression of the SYNGR3 gene and/or gene product is greater than the reference level of expression of the SYNGR3 gene and/or gene product; and
e) treating the HNSCC in the subject with a protocol for patients with that are infected by HPV-positive HNSCC.

16. The method of claim 15, wherein the sample is from a HNSCC tumor in the subject.

17. The method of claim 15, wherein the gene product comprises one or more of a mRNA and a protein.

18. The method of claim 15, further comprising the steps of:
detecting a level of expression of a p16 gene and/or gene product in the sample, and
comparing a level of p16 gene and/or gene product in the sample with the reference level of expression of a p16 gene and/or gene product;
wherein the subject is diagnosed as positive for HPV when the level of expression of the SYNGR3 gene and/or gene product is greater than the reference level of expression of the SYNGR3 gene and/or gene product and the level of the p16 gene and/or gene product is greater than the reference level of expression of the p16 gene and/or gene product.

19. The method of claim 15, wherein the detecting step comprises performing in situ hybridization (ISH) and/or immunohistochemistry (IHC) of SYNGR3 and/or p16 gene and/or gene product on the sample of the subject.

20. The method of claim 15, wherein the comparing step comprises quantifying the gene and/or gene product expression level, comparing the quantified gene and/or gene product expression level between that of the subject and reference level of expression of p16 gene and/or gene product, and optionally, generating a ROC curve.

21. The method of claim 15, further comprising determining an HPV status for the subject via HPV16.
